SEQUENCE LISTING (35 sequences)

SEQ ID NO: 1 - protein, 12 aa, Artificial Sequence
  Light and heavy chain Ipilimumab - CDRL1
RASQSVGSSYLA

SEQ ID NO: 2 - protein, 7 aa, Artificial Sequence
  Light and heavy chain Ipilimumab - CDRL2
GAFSRAT

SEQ ID NO: 3 - protein, 9 aa, Artificial Sequence
  Light and heavy chain Ipilimumab - CDRL3
QQYGSSPWT

SEQ ID NO: 4 - protein, 5 aa, Artificial Sequence
  Light and heavy chain Ipilimumab - CDRH1
SYTMH

SEQ ID NO: 5 - protein, 17 aa, Artificial Sequence
  Light and heavy chain Ipilimumab - CDRH2
FISYDGNNKYYADSVKG

SEQ ID NO: 6 - protein, 9 aa, Artificial Sequence
  Light and heavy chain Ipilimumab - CDRH3
TGWLGPFDY

SEQ ID NO: 7 - protein, 118 aa, Artificial Sequence
  Heavy chain VR - Ipilimumab
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 8 - protein, 108 aa, Artificial Sequence
  Light chain VR - Ipilimumab
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 9 - protein, 448 aa, Artificial Sequence
  Mature heavy chain and the mature light chain of Ipilimumab
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Artificial Sequence
  Mature heavy chain and the mature light chain of Ipilimumab
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, Artificial Sequence
  Monoclonal antibody CDRH1
GFTFSDNW

SEQ ID NO: 12 - protein, 10 aa, Artificial Sequence
  Monoclonal antibody CDRH2
IRNKPYNYET

SEQ ID NO: 13 - protein, 6 aa, Artificial Sequence
  Monoclonal antibody CDRH3
TAQFAY

SEQ ID NO: 14 - protein, 6 aa, Artificial Sequence
  Monoclonal antibody CDRL1
ENIYGG

SEQ ID NO: 15 - protein, 3 aa, Artificial Sequence
  Monoclonal antibody CDRL2
GAT

SEQ ID NO: 16 - protein, 9 aa, Artificial Sequence
  Monoclonal antibody CDRL3
QNVLRSPFT

SEQ ID NO: 17 - protein, 9 aa, Artificial Sequence
  Monoclonal antibody CDRL3
QNVLSRHPG

SEQ ID NO: 18 - protein, 9 aa, Artificial Sequence
  Monoclonal antibody CDRL3
QNVLSSRPG

SEQ ID NO: 19 - protein, 115 aa, Artificial Sequence
  8D2/8D2 (RE) VH
EVKLDETGGGLVQPGRPMKLSCVASGFTFSDNWMNWVRQSPEKGLEWLAQIRNKPYNYETYYSDSVKGRFTISRDDSKSSVYLQMNNLRGEDMGIYYCTAQFAYWGQGTLVTVSA

SEQ ID NO: 20 - protein, 106 aa, Artificial Sequence
  8D2/8D2 (RE) VL
DIQMTQSPASLSASVGETVTITCGTSENIYGGLNWYQRKQGKSPQLLIFGATNLADGMSSRFSGSGSGRQYSLKISSLHPDDVATYYCQNVLRSPFTFGSGTKLEI

SEQ ID NO: 21 - protein, 115 aa, Artificial Sequence
  8D2H1L1 VH
EVQLVESGGGLVQPGGSMRLSCAASGFTFSDNWMNWVRQAPGKGLEWLAQIRNKPYNYETYYSDSVKGRFTISRDDSKNSVYLQMNSLKTEDTGVYYCTAQFAYWGQGTLVTVSS

SEQ ID NO: 22 - protein, 107 aa, Artificial Sequence
  8D2H1L1 VL
DIQMTQSPSSLSASVGDRVTITCRTSENIYGGLNWYQRKQGKSPKLLIYGATNLASGMSSRFSGSGSGTDYTLKISSLHPDDVATYYCQNVLRSPFTFGSGTKLEIK

SEQ ID NO: 23 - protein, 115 aa, Artificial Sequence
  8D2H2L2 VH
EVQLVESGGGLVQPGGSMRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 24 - protein, 107 aa, Artificial Sequence
  8D2H2L2 VL
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIK

SEQ ID NO: 25 - protein, 115 aa, Artificial Sequence
  8D2H2L2 VH - VARIANT 1
EVQLVESGGGLVQPGGSLRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSS

SEQ ID NO: 26 - protein, 115 aa, Artificial Sequence
  8D3H3L3 VL
EVQLVESGGGLVQPGGSLRLSCAASGFTFSDNWMNWVRQAPGKGLEWVAQIRNKPYNYETEYAASVKGRFTISRDDSKNSAYLQMNSLKTEDTAVYYCTAQFAYWGQGTLVTVSS

SEQ ID NO: 27 - protein, 107 aa, Artificial Sequence
  8D3H3L3 VL
DIQMTQSPSSLSASVGDRVTITCRASENIYGGLNWYQQKPGKAPKLLIYGATSLASGVPSRFSGSGSGTDYTLTISSLQPEDFATYYCQNVLRSPFTFGSGTKLEIK

SEQ ID NO: 28 - protein, 115 aa, Artificial Sequence; identical to SEQ ID NO: 23
  8D2H2L15 VH

SEQ ID NO: 29 - protein, 107 aa, Artificial Sequence
  8D2H2L15 VL
DIQMTQSPSSLSASVGDRVTITCRTSENIYGGLNWYQRKPGKSPKLLIYGATNLASGVSSRFSGSGSGTDYTLTISSLQPEDVATYYCQNVLSRHPGFGSGTKLEIK

SEQ ID NO: 30 - protein, 115 aa, Artificial Sequence; identical to SEQ ID NO: 23
  8D2H2L17 VH

SEQ ID NO: 31 - protein, 107 aa, Artificial Sequence
  8D2H2L17 VL
DIQMTQSPSSLSASVGDRVTITCRTSENIYGGLNWYQRKPGKSPKLLIYGATNLASGVSSRFSGSGSGTDYTLTISSLQPEDVATYYCQNVLSSRPGFGSGTKLEIK

SEQ ID NO: 32 - protein, 445 aa, Artificial Sequence
  8D2H2L2 Full Heavy Chain - VARIANT 1
EVQLVESGGGLVQPGGSLRLSCAASGFTFSDNWMNWVRQAPGKGLEWLAQIRNKPYNYETYYSASVKGRFTISRDDSKNSVYLQMNSLKTEDTGVYYCTAQFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 214 aa, Artificial Sequence
  8D2H2L2 Full Light Chain - VARIANT 1
DIQMTQSPSSLSASVGDRVTITCRTSENIYGGLNWYQRKPGKSPKLLIYGATNLASGVSSRFSGSGSGTDYTLTISSLQPEDVATYYCQNVLRSPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 7 aa, Artificial Sequence
  Phospholipase B-Like 2 (PLBL2) peptide
LTFPTGR

SEQ ID NO: 35 - protein, 7 aa, Artificial Sequence
  Lipoprotein lipase (LPL) peptide
LVAALYK

(19) United States
(12) Patent Application Publication
(10) Pub. No.: US 2024/0115701 A1
(43) Pub. Date: Apr. 11, 2024

(54) METHODS AND COMPOSITIONS COMPRISING AN ANTI-CTLA4 MONOCLONAL ANTIBODY WITH REDUCED HOST CELL PROTEINS AND INCREASED POLYSORBATE-80 STABILITY

(71) Applicant: Merck Sharp & Dohme Corp., Rahway, NJ (US)

(72) Inventors: Rebecca A. Chmielowski, Clark, NJ (US); Francis K. Insaidoo, North Brunswick, NJ (US); Justin B. Miller, Hoboken, NJ (US); David J. Roush, Colts Neck, NJ (US); Darshini Shah, Somerset, NJ (US); John P. Welsh, Charlottesville, VA (US)

(73) Assignee: Merck Sharp & Dohme Corp., Rahway, NJ (US)

(21) Appl. No.: 17/642,870

(22) PCT Filed: Sep. 18, 2020
(86) PCT No.: PCT/US2020/051355

§ 371 Date: Mar. 14, 2022

Related U.S. Application Data
(60) Provisional application No. 62/904,331, filed on Sep. 23, 2019.

(51) Int. Cl. A61K 39/395; A61K 47/20; A61K 47/26; C07K 16/06; C07K 16/28

(52) U.S. Cl. CPC A61K 39/39591; A61K 47/20; A61K 47/26; C07K 16/065; C07K 16/2818

(57) ABSTRACT

Provided herein are methods of separating host cell lipases from a production protein in chromatographic processes and methods of improving polysorbate-80 stability in a production protein formulation by separating host cell lipases from the production protein using chromatographic processes. Also provided herein are compositions comprising antibodies or antigen binding fragments thereof that bind to cytotoxic T lymphocyte associated antigen 4 (CTLA4). In another aspect, such compositions further comprise a reduced level of host cell proteins and/or increased level of polysorbate-80 (PS-80) stability.

### CROSS-REFERENCE TO RELATED APPLICATIONS

This application is a national stage entry under 35 U.S.C. § 371 of PCT/US2020/051355 filed Sep. 18, 2020, which claims priority from and the benefit of U.S. Provisional Application No. 62/904,331, filed Sep. 23, 2019.

### I. FIELD

Provided herein are methods of separating host cell proteins (HCP) (e.g., lipases) from a production protein (e.g., monoclonal antibody) in chromatographic processes. Also provided herein are methods of improving polysorbate-80 (PS-80) stability in a production protein formulation (e.g., drug substance formulation or drug product formulation) by separating HCP (e.g., lipases) from the production protein (e.g., monoclonal antibody) using chromatographic processes. Also provided herein are compositions comprising antibodies or antigen binding fragments thereof that bind to cytotoxic T lymphocyte associated antigen 4 (CTLA4). In another aspect, such compositions further comprise a reduced level of host cell proteins with increased level of PS-80 stability.

### REFERENCE TO SEQUENCE LISTING SUBMITTED ELECTRONICALLY

The sequence listing of the present application is submitted electronically via EFS-Web as an ASCII formatted sequence listing with a file name “24815WOPCT-SEQLIST-25SEP2023.txt”, creation date of Sep. 25, 2023, and a size of 30.7 Kb. This sequence listing submitted via EFS-Web is part of the specification and is herein incorporated by reference in its entirety.

### II. BACKGROUND OF THE INVENTION

In bioprocessing and manufacturing of production proteins (e.g., monoclonal antibodies), HCP (e.g., lipases) constitute part of the impurities that are often difficult to remove from the production proteins. Such impurities can cause various issues in the safety and efficacy of biopharmaceuticals. Regulatory agencies throughout the world require that biopharmaceutical products meet certain acceptance criteria, including the level of impurities and tests for detection and quantification of impurities. Thus, it is desirable to develop efficient and effective processes to remove HCP (e.g., lipases) from production proteins (e.g., monoclonal antibodies).

CTLA4 mAbs or CTLA4 ligands can prevent CTLA4 from binding to its native ligands, thereby blocking the transduction of the T cell negative regulating signal by CTLA4 and enhancing the responsiveness of T cells to various antigens. In this aspect, results from in vivo and in vitro studies are substantially in concert. CTLA-4 was validated as an immunotherapeutic target after FDA approval of Ipilimumab for human use, either as monotherapy for melanoma, or as part of combination therapy with the anti-PD-1 antibody, Nivolumab, in melanoma, renal cancer, colorectal cancer with microsatellite instability. (Zhang, P., et al. Mechanism and Immune Landscape Based ranking of Therapeutic Responsiveness of 22 Major Human Cancers to Next Generation Anti-CTLA-4 Antibodies, Cancers, 12(2), 284). There are some CTLA4 mAbs being tested in clinical trials for treating prostate cancer, bladder cancer, colorectal cancer, cancer of gastrointestinal tract, liver cancer, malignant melanoma, etc. (Grosso et al., CTLA-4 blockade in tumor models: an overview of preclinical and translational research. Cancer Immun. 13:5 (2013)).

### III. SUMMARY

The present disclosure provides methods of separating HCP (e.g., lipases) from a production protein (e.g., monoclonal antibody) in chromatographic processes as well as methods of improving PS-80 stability in a production protein formulation (e.g., drug substance formulation or drug product formulation) by separating HCP (e.g., lipases) from a production protein (e.g., monoclonal antibody) using chromatographic processes. The disclosure is based, at least in part, on the discovery that the HCP (e.g., lipases) and the production protein (e.g., monoclonal antibody) can be sufficiently separated under operating conditions where the separation factor (α) between the two proteins and/or the partition coefficient (K_{P}) for the HCP (e.g., lipase) reach certain ranges of numeric values.

In one aspect, the production protein is an anti-CTLA4 antibody.

In one aspect, provided herein is a method of separating a host cell lipase from an anti-CTLA4 antibody, or antigen binding fragment thereof, through a chromatographic process, comprising:
(a) passing a load fluid comprising the lipase and the anti-CTLA4 antibody, or antigen binding fragment thereof, through a chromatographic resin under a loading operating condition; and
(b) collecting the anti-CTLA4 antibody, or antigen binding fragment thereof, in a flowthrough;
   wherein separation factor (α) is the ratio of the partition coefficient (K_{P}) for the lipase to the K_{P} for the anti-CTLA4 antibody, or antigen binding fragment thereof, and wherein log α is larger than 0.5 under the loading operating condition.

In certain embodiments, log α is larger than 1.0 under the loading operating condition.

In some embodiments, the log K_{P} for the lipase is larger than 1.0 under the loading operating condition. In other embodiments, the log K_{P} for the lipase is larger than 1.5 under the loading operating condition.

In certain embodiments, log α is larger than 0.5 and the log K_{P} for the lipase is larger than 1.0 under the loading operating condition. In some embodiments, log α is larger than 0.5 and the log K_{P} for the lipase is larger than 1.5 under the loading operating condition. In other embodiments, log α is larger than 1.0 and the log K_{P} for the lipase is larger than 1.0 under the loading operating condition. In yet other embodiments, log α is larger than 1.0 and the log K_{P} for the lipase is larger than 1.5 under the loading operating condition.

In another aspect, provided herein is a method of separating a host cell lipase from an anti-CTLA4 antibody, or antigen binding fragment thereof, through a chromatographic process, comprising:
(a) passing a load fluid comprising the lipase and the anti-CTLA4 antibody, or antigen binding fragment thereof, through a chromatographic resin; and
(b) eluting the anti-CTLA4 antibody, or antigen binding fragment thereof, from the chromatographic resin with an elution solution under an elution operating condition; wherein α is the ratio of K_{P} for the lipase to the K_{P} for the anti-CTLA4 antibody, or antigen binding fragment thereof, and wherein log α is larger than 0.5 under the elution operating condition.

In certain embodiments, log α is larger than 1.0 under the elution operating condition.

In some embodiments, the log K_{P} for the lipase is larger than 1.0 under the elution operating condition. In other embodiments, the log K_{P} for the lipase is larger than 1.5 under the elution operating condition.

In certain embodiments, log α is larger than 0.5 and the log K_{P} for the lipase is larger than 1.0 under the elution operating condition. In some embodiments, log α is larger than 0.5 and the log K_{P} for the lipase is larger than 1.5 under the elution operating condition. In other embodiments, log α is larger than 1.0 and the log K_{P} for the lipase is larger than 1.0 under the elution operating condition. In yet other embodiments, log α is larger than 1.0 and the log K_{P} for the lipase is larger than 1.5 under the elution operating condition.

In some embodiments of various methods provided herein, the lipase is a Chinese Hamster Ovary (CHO) cell lipase.

In certain embodiments, the lipase is selected from the group consisting of phospholipase B-like 2 (PLBL2), lipoprotein lipase (LPL), lysosomal phospholipase A2 (LPLA2), phospholipase A2 VII (LP-PLA2), and lysosomal acid lipase A (LAL). In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

In certain embodiments, the CHO cell lipase is selected from the group consisting of PLBL2, LPL, LPLA2, LP-PLA2, and LAL. In one embodiment, the CHO cell lipase is PLBL2. In another embodiment, the CHO cell lipase is LPL. In yet another embodiment, the CHO cell lipase is LPLA2. In one embodiment, the CHO cell lipase is LP-PLA2. In another embodiment, the CHO cell lipase is LAL. In still another embodiment, the CHO cell lipase includes two, three, four, five, six, seven, eight, nine, ten, or more different CHO cell lipases. In yet still another embodiment, the CHO cell lipase includes two, three, four, or five different CHO cell lipases selected from the group consisting of PLBL2, LPL, LPLA2, LP-PLA2, and LAL. In one embodiment, the CHO cell lipase includes PLBL2 and LPL. In another embodiment, the CHO cell lipase includes PLBL2 and LPLA2. In yet another embodiment, the CHO cell lipase includes PLBL2 and LP-PLA2. In still another embodiment, the CHO cell lipase includes PLBL2 and LAL. In one embodiment, the CHO cell lipase includes LPL and LPLA2. In another embodiment, the CHO cell lipase includes LPL and LP-PLA2. In yet another embodiment, the CHO cell lipase includes LPL and LAL. In still another embodiment, the CHO cell lipase includes LPLA2 and LP-PLA2. In one embodiment, the CHO cell lipase includes LPLA2 and LAL. In another embodiment, the CHO cell lipase includes LP-PLA2 and LAL. In yet another embodiment, the CHO cell lipase includes PLBL2, LPL, and LPLA2. In still another embodiment, the CHO cell lipase includes PLBL2, LPL, and LP-PLA2. In one embodiment, the CHO cell lipase includes PLBL2, LPL, and LAL. In another embodiment, the CHO cell lipase includes PLBL2, LPLA2, and LP-PLA2. In yet another embodiment, the CHO cell lipase includes PLBL2, LPLA2, and LAL. In still another embodiment, the CHO cell lipase includes PLBL2, LP-PLA2, and LAL. In one embodiment, the CHO cell lipase includes LPL, LPLA2, and LP-PLA2. In another embodiment, the CHO cell lipase includes LPL, LPLA2, and LAL. In yet another embodiment, the CHO cell lipase includes LPL, LP-PLA2, and LAL. In still another embodiment, the CHO cell lipase includes LPLA2, LP-PLA2, and LAL. In one embodiment, the CHO cell lipase includes PLBL2, LPL, LPLA2, and LP-PLA2. In another embodiment, the CHO cell lipase includes PLBL2, LPL, LPLA2, and LAL. In yet another embodiment, the CHO cell lipase includes PLBL2, LPL, LP-PLA2, and LAL. In still another embodiment, the CHO cell lipase includes PLBL2, LPLA2, LP-PLA2, and LAL. In yet still another embodiment, the CHO cell lipase includes PLBL2, LPL, LPLA2, LP-PLA2, and LAL.

In some embodiments of various methods provided herein, the chromatographic resin is an ion exchange (IEX) resin. In other embodiments, the chromatographic resin is a hydrophobic interaction (HIC) resin. In one embodiment, the IEX resin is a cation exchange (CEX) resin. In another embodiment, the CEX resin is a mixed mode CEX resin. In yet another embodiment, the IEX resin is an anion exchange (AEX) resin. In still another embodiment, the AEX resin is a mixed mode AEX resin.

In certain embodiments of various methods using a CEX resin or a mixed mode CEX resin, the pH of the operating condition is below about 6.0. In some embodiments of various methods using a CEX resin or a mixed mode CEX resin, the pH of the operating condition is below about 5.5. In other embodiments of various methods using a CEX resin or a mixed mode CEX resin, the pH of the operating condition is below about 5.0. In yet other embodiments of various methods using a CEX resin or a mixed mode CEX resin, the pH of the operating condition is from about 4.5 to about 5.5. In still other embodiments of various methods using a CEX resin or a mixed mode CEX resin, the pH of the operating condition is from about 4.5 to about 5.0. In certain embodiments of various methods using a CEX resin or a mixed mode CEX resin, the pH of the operating condition is from about 5.0 to about 5.5. In some embodiments of various methods using a CEX resin or a mixed mode CEX resin, the pH of the operating condition is from about 4.9 to about 5.3.

In certain embodiments of various methods using an AEX resin or a mixed mode AEX resin, the pH of the operating condition is above about 6.5. In some embodiments of various methods using an AEX resin or a mixed mode AEX resin, the pH of the operating condition is above about 6.9. In other embodiments of various methods using an AEX resin or a mixed mode AEX resin, the pH of the operating condition is above about 7.2. In yet other embodiments of various methods using an AEX resin or a mixed mode AEX resin, the pH of the operating condition is from about 6.9 to about 7.9. In still other embodiments of various methods using an AEX resin or a mixed mode AEX resin, the pH of the operating condition is from about 7.2 to about 7.5. In certain embodiments of various methods using an AEX resin or a mixed mode AEX resin, the pH of the operating condition is from about 7.5 to about 7.8.

In certain embodiments of various methods provided herein, the operating condition further comprises modulating the ionic strength and/or conductivity of the operating solution by adding a salt. In one embodiment, the operating condition further comprises modulating the ionic strength of the operating solution by adding a salt. In another embodiment, the operating condition further comprises modulating the conductivity of the operating solution by adding a salt. In yet another embodiment, the operating condition further comprises modulating the ionic strength and conductivity of the operating solution by adding a salt. In some embodiments, the effect of adding a salt is to achieve the desired log α. In other embodiments, the effect of adding a salt is to achieve the desired log K_{P} for the lipase. In yet other embodiments, the effect of adding a salt is to achieve the desired log α and the desired log K_{P} for the lipase.

In some embodiments, the salt in the operating solution is selected from the group consisting of sodium chloride, sodium acetate, sodium phosphate, ammonium sulfate, sodium sulfate, and Tris-HCl. In one embodiment, the salt is sodium chloride. In another embodiment, the salt is sodium acetate. In yet another embodiment, the salt is sodium phosphate. In still another embodiment, the salt is ammonium sulfate. In one embodiment, the salt is sodium sulfate. In another embodiment, the salt is Tris-HCl.

In a specific embodiment, the concentration of sodium chloride in the operating solution is from about 100 mM to about 225 mM, the chromatographic resin is CEX, and the pH of the operating condition is from about 5.0 to about 6.0.

In another specific embodiment, the concentration of sodium chloride in the operating solution is from about 150 mM to about 180 mM, the chromatographic resin is CEX, and the pH of the operating condition is from about 5.0 to about 6.0.

In yet another specific embodiment, the concentration of sodium acetate in the operating solution is from about 100 mM to about 200 mM, the chromatographic resin is AEX; the pH of the operating condition is from about 6.9 to about 7.8.

In still another specific embodiment, the concentration of sodium sulfate in the operating solution is from about 500 mM to about 620 mM, the chromatographic resin is HIC, and the pH of the operating condition is about 7.

In yet still another specific embodiment, the concentration of sodium sulfate in the operating solution is from about 510 mM to about 560 mM, the chromatographic resin is HIC, and the pH of the operating condition is about 7.

In yet another aspect, provided herein is a method of separating PLBL2 and improving PS-80 stability from an anti-CTLA4 antibody, or antigen binding fragment thereof, through a mixed mode AEX chromatographic process comprising:
(a) passing a load fluid comprising PLBL2 and the anti-CTLA4 antibody, or antigen binding fragment thereof, through a mixed mode AEX resin; and
(b) collecting the anti-CTLA4 antibody, or antigen binding fragment thereof, in a flowthrough;
   wherein the pH of the load fluid is from about pH 7.2 to about pH 7.6, and wherein the load fluid does not comprise a salt.

In still another aspect, provided herein is a method of separating PLBL2 and improving PS-80 stability from an anti-CTLA4 antibody, or antigen binding fragment thereof, through a CEX chromatographic process comprising:
(a) passing a load fluid comprising PLBL2 and the anti-CTLA4 antibody, or antigen binding fragment thereof, through a CEX resin; and
(b) eluting the anti-CTLA4 antibody, or antigen binding fragment thereof, from the CEX resin with an elution solution;
   wherein the pH of the elution solution is from about pH 4.9 to about pH 5.3, and wherein the elution solution further comprises from about 120 mM to about 175 mM sodium chloride.

In one embodiment, the method of separating PLBL2 and improving PS-80 stability from an anti-CTLA4 antibody, or antigen binding fragment thereof, through a CEX chromatographic process comprises:
(a) passing a load fluid comprising PLBL2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is about pH 5.1, and wherein the elution solution further comprises about 150 mM sodium chloride.

In yet another embodiment, the method of separating PLBL2 and improving PS-80 stability from an anti-CTLA4 antibody, or antigen binding fragment thereof, through a CEX chromatographic process comprises:
(a) passing a load fluid comprising PLBL2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is about pH 5.1, and wherein the elution solution further comprises about 165 mM sodium chloride.

In still another aspect, provided herein is a method of separating LPLA2 and improving PS-80 stability from an anti-CTLA4 antibody, or antigen binding fragment thereof, through a CEX chromatographic process, comprising:
(a) passing a load fluid comprising LPLA2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is from about pH 5.0 to about pH 5.4, and wherein the elution solution further comprises from about 150 mM to about 275 mM sodium chloride.

In one embodiment, the method of separating LPLA2 and improving PS-80 stability from an anti-CTLA4 antibody, or antigen binding fragment thereof, through a CEX chromatographic process comprises:
(a) passing a load fluid comprising LPLA2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is about pH 5.1, and wherein the elution solution further comprises about 150 mM sodium chloride.

In another embodiment, the method of separating LPLA2 and improving PS-80 stability from an anti-CTLA4 antibody, or antigen binding fragment thereof, through a CEX chromatographic process comprises:
(a) passing a load fluid comprising LPLA2 and the anti-CTLA4 antibody, or antigen binding fragment thereof, through a CEX resin; and
(b) eluting the anti-CTLA4 antibody, or antigen binding fragment thereof, from the CEX resin with an elution solution;
   wherein the pH of the elution solution is about pH 5.1, and wherein the elution solution further comprises about 200 mM sodium chloride.

In yet another embodiment, the method of separating LPLA2 and improving PS-80 stability from an anti-CTLA4 antibody, or antigen binding fragment thereof, through a CEX chromatographic process comprises:
(a) passing a load fluid comprising LPLA2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is about pH 5.1, and wherein the elution solution further comprises about 250 mM sodium chloride.

In more embodiments of the various methods provided herein, the load fluid is an eluate from a prior chromatographic process. In one embodiment, the prior chromatographic process comprises an affinity chromatography. In another embodiment, the prior chromatographic process comprises an affinity chromatography followed by a non-affinity chromatography. In yet another embodiment, the affinity chromatography is a protein A chromatography. In still another embodiment, the non-affinity chromatography is an AEX chromatography. In yet still another embodiment, the prior chromatographic process comprises a protein A chromatography followed by an AEX chromatography.

In yet still another aspect, provided herein is a method of improving PS-80 stability in an anti-CTLA4 antibody, or antigen binding fragment thereof, formulation, comprising:
(a) passing a load fluid comprising a host cell lipase and an anti-CTLA4 antibody, or antigen binding fragment thereof, through a chromatographic resin under a loading operating condition;
(b) collecting the anti-CTLA4 antibody, or antigen binding fragment thereof, in a flowthrough;
(c) additionally, passing the flowthrough product comprising a host cell lipase through an additional chromatographic resin;
(d) collecting the anti-CTLA4 antibody, or antigen binding fragment thereof, and
(e) formulating the anti-CTLA4 antibody, or antigen binding fragment thereof, so that the anti-CTLA4 antibody, or antigen binding fragment thereof, formulation comprises PS-80;
   wherein separation factor (α) is the ratio of the partition coefficient (K_{P}) for the lipase to the K_{P} for the anti-CTLA4 antibody, or antigen binding fragment thereof, and wherein log α is larger than 0.5 under the loading operating condition.

In yet still another aspect, provided herein is a method of improving PS-80 stability in an anti-CTLA4 antibody, or antigen binding fragment thereof, formulation, comprising:
(f) passing a load fluid comprising PLBL2 and an anti-CTLA4 antibody, or antigen binding fragment thereof, through a chromatographic resin under a loading operating condition;
(g) collecting the anti-CTLA4 antibody, or antigen binding fragment thereof, in a flowthrough;
(h) additionally, passing the flowthrough product comprising PLBL2 through an additional chromatographic resin;
(i) collecting the anti-CTLA4 antibody, or antigen binding fragment thereof, and
(j) formulating the anti-CTLA4 antibody, or antigen binding fragment thereof, so that the anti-CTLA4 antibody, or antigen binding fragment thereof, formulation comprises PS-80;
   wherein separation factor (α) is the ratio of the partition coefficient (K_{P}) for the lipase to the K_{P} for the anti-CTLA4 antibody, or antigen binding fragment thereof, and wherein log α is larger than 0.5 under the loading operating condition.

In still another aspect, provided herein is a method of decreasing PS-80 degradation in an anti-CTLA4 antibody, or antigen binding fragment thereof, composition comprising:
(a) passing a load fluid comprising a host cell lipase and an anti-CTLA4 antibody, or antigen binding fragment thereof, through a chromatographic resin under a loading operating condition;
(b) collecting the anti-CTLA4 antibody, or antigen binding fragment thereof, in a flowthrough;
(c) passing the flowthrough product comprising a host cell lipase through an additional chromatographic resin;
(d) collecting the anti-CTLA4 antibody, or antigen binding fragment thereof, and
(e) formulating the anti-CTLA4 antibody, or antigen binding fragment thereof, so that the anti-CTLA4 antibody, or antigen binding fragment thereof, formulation comprises PS-80;

In certain embodiments, log α is larger than 1.0 under the loading operating condition.

In some embodiments, the log K_{P} for the lipase is larger than 1.0 under the loading operating condition. In other embodiments, the log K_{P} for the lipase is larger than 1.5 under the loading operating condition.

In certain embodiments, log α is larger than 0.5 and the log K_{P} for the lipase is larger than 1.0 under the loading operating condition. In some embodiments, log α is larger than 0.5 and the log K_{P} for the lipase is larger than 1.5 under the loading operating condition. In other embodiments, log α is larger than 1.0 and the log K_{P} for the lipase is larger than 1.0 under the loading operating condition. In yet other embodiments, log α is larger than 1.0 and the log K_{P} for the lipase is larger than 1.5 under the loading operating condition.

In another aspect, provided herein is a method of improving PS-80 stability in an anti-CTLA4 antibody, or antigen binding fragment thereof, formulation, comprising:
(a) passing a load fluid comprising a host cell lipase and the anti-CTLA4 antibody, or antigen binding fragment thereof, through a chromatographic resin;
(b) eluting the anti-CTLA4 antibody, or antigen binding fragment thereof, from the chromatographic resin with an elution solution under an elution operating condition; and
(c) formulating the anti-CTLA4 antibody, or antigen binding fragment thereof, so that the anti-CTLA4 antibody, or antigen binding fragment thereof, formulation is a PS-80-containing solution;
   wherein α is the ratio of K_{P} for the lipase to the K_{P} for the anti-CTLA4 antibody, or antigen binding fragment thereof, and wherein log α is larger than 0.5 under the elution operating condition.

In certain embodiments, log α is larger than 1.0 under the elution operating condition.

In some embodiments, the log K_{P} for the lipase is larger than 1.0 under the elution operating condition. In other embodiments, the log K_{P} for the lipase is larger than 1.5 under the elution operating condition.

In certain embodiments, log α is larger than 0.5 and the log K_{P} for the lipase is larger than 1.0 under the elution operating condition. In some embodiments, log α is larger than 0.5 and the log K_{P} for the lipase is larger than 1.5 under the elution operating condition. In other embodiments, log α is larger than 1.0 and the log K_{P} for the lipase is larger than 1.0 under the elution operating condition. In yet other embodiments, log α is larger than 1.0 and the log K_{P} for the lipase is larger than 1.5 under the elution operating condition.

In some embodiments of various methods provided herein, the lipase is a Chinese Hamster Ovary (CHO) cell lipase.

In certain embodiments, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

In certain embodiments, the CHO cell lipase is selected from the group consisting of PLBL2, LPL, LPLA2, LP-PLA2, and LAL. In one embodiment, the CHO cell lipase is PLBL2. In another embodiment, the CHO cell lipase is LPL. In yet another embodiment, the CHO cell lipase is LPLA2. In one embodiment, the CHO cell lipase is LP-PLA2. In another embodiment, the CHO cell lipase is LAL. In still another embodiment, the CHO cell lipase includes two, three, four, five, six, seven, eight, nine, ten, or more different CHO cell lipases. In yet still another embodiment, the CHO cell lipase includes two, three, four, or five different CHO cell lipases selected from the group consisting of PLBL2, LPL, LPLA2, LP-PLA2, and LAL. In one embodiment, the CHO cell lipase includes PLBL2 and LPL. In another embodiment, the CHO cell lipase includes PLBL2 and LPLA2. In yet another embodiment, the CHO cell lipase includes PLBL2 and LP-PLA2. In still another embodiment, the CHO cell lipase includes PLBL2 and LAL. In one embodiment, the CHO cell lipase includes LPL and LPLA2. In another embodiment, the CHO cell lipase includes LPL and LP-PLA2. In yet another embodiment, the CHO cell lipase includes LPL and LAL. In still another embodiment, the CHO cell lipase includes LPLA2 and LP-PLA2. In one embodiment, the CHO cell lipase includes LPLA2 and LAL. In another embodiment, the CHO cell lipase includes LP-PLA2 and LAL. In yet another embodiment, the CHO cell lipase includes PLBL2, LPL, and LPLA2. In still another embodiment, the CHO cell lipase includes PLBL2, LPL, and LP-PLA2. In one embodiment, the CHO cell lipase includes PLBL2, LPL, and LAL. In another embodiment, the CHO cell lipase includes PLBL2, LPLA2, and LP-PLA2. In yet another embodiment, the CHO cell lipase includes PLBL2, LPLA2, and LAL. In still another embodiment, the CHO cell lipase includes PLBL2, LP-PLA2, and LAL. In one embodiment, the CHO cell lipase includes LPL, LPLA2, and LP-PLA2. In another embodiment, the CHO cell lipase includes LPL, LPLA2, and LAL. In yet another embodiment, the CHO cell lipase includes LPL, LP-PLA2, and LAL. In still another embodiment, the CHO cell lipase includes LPLA2, LP-PLA2, and LAL. In one embodiment, the CHO cell lipase includes PLBL2, LPL, LPLA2, and LP-PLA2. In another embodiment, the CHO cell lipase includes PLBL2, LPL, LPLA2, and LAL. In yet another embodiment, the CHO cell lipase includes PLBL2, LPL, LP-PLA2, and LAL. In still another embodiment, the CHO cell lipase includes PLBL2, LPLA2, LP-PLA2, and LAL. In yet still another embodiment, the CHO cell lipase includes PLBL2, LPL, LPLA2, LP-PLA2, and LAL.

In some embodiments of various methods provided herein, the chromatographic resin is an IEX resin. In other embodiments, the chromatographic resin is a HIC resin. In one embodiment, the IEX resin is a CEX resin. In another embodiment, the CEX resin is a mixed mode CEX resin. In yet another embodiment, the IEX resin is an AEX resin. In still another embodiment, the AEX resin is a mixed mode AEX resin.

In certain embodiments of various methods using a CEX resin or a mixed mode CEX resin, the pH of the operating condition is below about 6.0. In some embodiments of various methods using a CEX resin or a mixed mode CEX resin, the pH of the operating condition is below about 5.5. In other embodiments of various methods using a CEX resin or a mixed mode CEX resin, the pH of the operating condition is below about 5.0. In yet other embodiments of various methods using a CEX resin or a mixed mode CEX resin, the pH of the operating condition is from about 4.5 to about 5.5. In still other embodiments of various methods using a CEX resin or a mixed mode CEX resin, the pH of the operating condition is from about 4.5 to about 5.0. In certain embodiments of various methods using a CEX resin or a mixed mode CEX resin, the pH of the operating condition is from about 5.0 to about 5.5. In some embodiments of various methods using a CEX resin or a mixed mode CEX resin, the pH of the operating condition is from about 4.9 to about 5.3.

In certain embodiments of various methods using an AEX resin or a mixed mode AEX resin, the pH of the operating condition is above about 6.5. In some embodiments of various methods using an AEX resin or a mixed mode AEX resin, the pH of the operating condition is above about 6.9. In other embodiments of various methods using an AEX resin or a mixed mode AEX resin, the pH of the operating condition is above about 7.2. In yet other embodiments of various methods using an AEX resin or a mixed mode AEX resin, the pH of the operating condition is from about 6.9 to about 7.9. In still other embodiments of various methods using an AEX resin or a mixed mode AEX resin, the pH of the operating condition is from about 7.2 to about 7.5. In certain embodiments of various methods using an AEX resin or a mixed mode AEX resin, the pH of the operating condition is from about 7.5 to about 7.8.

In certain embodiments of various methods provided herein, the operating condition further comprises modulating the ionic strength and/or conductivity of the operating solution by adding a salt. In one embodiment, the operating condition further comprises modulating the ionic strength of the operating solution by adding a salt. In another embodiment, the operating condition further comprises modulating the conductivity of the operating solution by adding a salt. In yet another embodiment, the operating condition further comprises modulating the ionic strength and conductivity of the operating solution by adding a salt. In some embodiments, the effect of adding a salt is to achieve the desired log α. In other embodiments, the effect of adding a salt is to achieve the desired log K_{P} for the lipase. In yet other embodiments, the effect of adding a salt is to achieve the desired log α and the desired log K_{P} for the lipase.

In some embodiments, the salt in the operating solution is selected from the group consisting of sodium chloride, sodium acetate, sodium phosphate, ammonium sulfate, sodium sulfate, and Tris-HCl. In one embodiment, the salt is sodium chloride. In another embodiment, the salt is sodium acetate. In yet another embodiment, the salt is sodium phosphate. In still another embodiment, the salt is ammonium sulfate. In one embodiment, the salt is sodium sulfate. In another embodiment, the salt is Tris-HCl.

In a specific embodiment, the concentration of sodium chloride in the operating solution is from about 100 mM to about 225 mM, the chromatographic resin is CEX, and the pH of the operating condition is from about 5.0 to about 6.0.

In another specific embodiment, the concentration of sodium chloride in the operating solution is from about 150 mM to about 180 mM, the chromatographic resin is CEX, and the pH of the operating condition is from about 5.0 to about 6.0.

In yet another specific embodiment, the concentration of sodium acetate in the operating solution is from about 100 mM to about 200 mM, the chromatographic resin is AEX; the pH of the operating condition is from about 6.9 to about 7.8.

In still another specific embodiment, the concentration of sodium sulfate in the operating solution is from about 500 mM to about 620 mM, the chromatographic resin is HIC, and the pH of the operating condition is about 7.

In yet still another specific embodiment, the concentration of sodium sulfate in the operating solution is from about 510 mM to about 560 mM, the chromatographic resin is HIC, and the pH of the operating condition is about 7.

In yet another aspect, provided herein is a method of improving PS-80 stability in an anti-CTLA4 antibody, or antigen binding fragment thereof, formulation, comprising:
(a) passing a load fluid comprising the anti-CTLA4 antibody or antigen binding fragment thereof, through a mixed mode AEX resin;
(b) collecting the anti-CTLA4 antibody, or antigen binding fragment thereof, in a flowthrough; and
(c) formulating the anti-CTLA4 antibody, or antigen binding fragment thereof, so that the anti-CTLA4 antibody, or antigen binding fragment thereof, formulation is a PS-80-containing solution;
   wherein the pH of the load fluid is from about pH 7.2 to about pH 7.6, and wherein the load fluid does not comprise a salt.

In still another aspect, provided herein is a method of improving PS-80 stability in an anti-CTLA4 antibody, or antigen binding fragment thereof, formulation, comprising:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a PS-80-containing solution;
   wherein the pH of the elution solution is from about pH 4.9 to about pH 5.3, and wherein the elution solution further comprises from about 120 mM to about 175 mM sodium chloride.

In one embodiment, the method of improving PS-80 stability in an anti-CTLA4 antibody, or antigen binding fragment thereof, formulation comprises:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a PS-80-containing solution;

wherein the pH of the elution solution is about pH 5.1, and wherein the elution solution further comprises about 150 mM sodium chloride.

In yet another embodiment, the method of improving PS-80 stability in an anti-CTLA4 antibody, or antigen binding fragment thereof, formulation comprises:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a PS-80-containing solution;

wherein the pH of the elution solution is about pH 5.1, and wherein the elution solution further comprises about 165 mM sodium chloride.

In still another aspect, provided herein is a method of improving PS-80 stability in an anti-CTLA4 antibody, or antigen binding fragment thereof, formulation, comprising:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody so that the anti-CTLA4 antibody, or antigen binding fragment thereof, formulation is a PS-80-containing solution;

wherein the pH of the elution solution is from about pH 5.0 to about pH 5.4, and wherein the elution solution further comprises from about 150 mM to about 275 mM sodium chloride.

In one embodiment, the method of improving PS-80 stability in an anti-CTLA4 antibody, or antigen binding fragment thereof, formulation comprises:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a PS-80-containing solution;

wherein the pH of the elution solution is about pH 5.1, and wherein the elution solution further comprises about 200 mM sodium chloride.

In another embodiment, the method of improving PS-80 stability in an anti-CTLA4 antibody, or antigen binding fragment thereof, formulation comprises:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a PS-80-containing solution;

wherein the pH of the elution solution is about pH 5.1, and wherein the elution solution further comprises about 250 mM sodium chloride.

In more embodiments of the various methods provided herein, the load fluid is an eluate from a prior chromatographic process. In one embodiment, the prior chromatographic process comprises an affinity chromatography. In another embodiment, the prior chromatographic process comprises an affinity chromatography followed by a non-affinity chromatography. In yet another embodiment, the affinity chromatography is a protein A chromatography. In still another embodiment, the non-affinity chromatography is an AEX chromatography. In yet still another embodiment, the prior chromatographic process comprises a protein A chromatography followed by an AEX chromatography.

In yet still another aspect, provided herein is a method of separating a host cell lipase from an anti-CTLA4 antibody, or antigen binding fragment thereof, through a CEX chromatographic process, comprising:
(a) passing a load fluid comprising the host cell lipase and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is from about pH 4.9 to about pH 5.4, and wherein the conductivity of the elution solution is from about 15 mS/cm to about 21 mS/cm.

In another aspect, provided herein is a method of improving PS-80 stability in an anti-CTLA4 antibody, or antigen binding fragment thereof, formulation, comprising:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation comprises PS-80;

wherein the pH of the elution solution is from about pH 4.9 to about pH 5.4, and wherein the conductivity of the elution solution is from about 15 mS/cm to about 21 mS/cm.

In yet still another aspect, provided herein is a method of separating a host cell lipase from an anti-CTLA4 antibody, or antigen binding fragment thereof, through a CEX chromatographic process, comprising:
(a) passing a load fluid comprising the host cell lipase and the anti-CTLA4 antibody, or antigen binding fragment thereof, through the CEX resin; and
(b) eluting the anti-CTLA4 antibody, or antigen binding fragment thereof, from the CEX resin with an elution solution;

wherein the pH of the elution solution is from about pH 4.9 to about pH 5.4, and wherein the elution solution further comprises from about 135 mM to about 195 mM sodium chloride.

In another aspect, provided herein is a method of improving PS-80 stability in an anti-CTLA4 antibody, or antigen binding fragment thereof, formulation, comprising:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation comprises PS-80;

wherein the pH of the elution solution is from about pH 4.9 to about pH 5.4, and wherein the elution solution further comprises from about 135 mM to about 195 mM sodium chloride.

In certain embodiments of various method described herein, the anti-CTLA4 antibody, or antigen binding fragment thereof, is a therapeutic protein.

In some embodiments of various method described herein, the anti-CTLA4 antibody is a monoclonal antibody.

In another aspect, provided herein is a pharmaceutical composition comprising a therapeutic protein and less than 1 ppm of a host cell lipase. In some embodiments, the pharmaceutical composition comprises a therapeutic protein and less than 0.1, 0.2, 0.3, 0.4, 0.5, 0.6, 0.7, 0.8, or 0.9 ppm of a host cell lipase. In one embodiment, the pharmaceutical composition comprises a therapeutic protein and less than 0.1 ppm of a host cell lipase. In another embodiment, the pharmaceutical composition comprises a therapeutic protein and less than 0.2 ppm of a host cell lipase. In yet another embodiment, the pharmaceutical composition comprises a therapeutic protein and less than 0.3 ppm of a host cell lipase. In still another embodiment, the pharmaceutical composition comprises a therapeutic protein and less than 0.4 ppm of a host cell lipase. In yet still another embodiment, the pharmaceutical composition comprises a therapeutic protein and less than 0.5 ppm of a host cell lipase. In one embodiment, the pharmaceutical composition comprises a therapeutic protein and less than 0.6 ppm of a host cell lipase. In another embodiment, the pharmaceutical composition comprises a therapeutic protein and less than 0.7 ppm of a host cell lipase. In yet another embodiment, the pharmaceutical composition comprises a therapeutic protein and less than 0.8 ppm of a host cell lipase. In still another embodiment, the pharmaceutical composition comprises a therapeutic protein and less than 0.9 ppm of a host cell lipase.

In various embodiments of the pharmaceutical compositions described herein, the level of the host cell lipase is measured by liquid chromatography-mass spectrometry (LC-MS).

In another aspect, provided herein is a pharmaceutical composition comprising a therapeutic protein with increased PS-80 stability, wherein the pharmaceutical composition has a decrease in PS-80 degradation.

In certain embodiments, the pharmaceutical composition is an eluate from a CEX chromatography comprising an anti-CTLA4 antibody, or antigen binding fragment thereof, using an elution solution selected from the group consisting of:
(a) an elution solution with a pH from about 4.9 to about 5.3, comprising from about 120 mM to about 175 mM sodium chloride;
(b) an elution solution with a pH of about 5.1, comprising about 150 mM sodium chloride;
(c) an elution solution with a pH of about 5.1, comprising about 165 mM sodium chloride;
(d) an elution solution with a pH from about 4.9 to about 5.4 and a conductivity from about 15 mS/cm to about 21 mS/cm;
(e) an elution solution with a pH from about 4.9 to about 5.4, comprising from about 135 mM to about 195 mM sodium chloride;
(f) an elution solution with a pH from about pH 5.0 to about pH 5.4, comprising from about 150 mM to about 275 mM sodium chloride;
(g) an elution solution with a pH of about 5.1, comprising about 200 mM sodium chloride; and
(h) an elution solution with a pH of about 5.1, comprising about 250 mM sodium chloride.

In one embodiment, the pharmaceutical composition is an eluate from a CEX chromatography using an elution solution with a pH from about 4.9 to about 5.3, comprising from about 120 mM to about 175 mM sodium chloride and an anti-CTLA4 antibody, or antigen binding fragment thereof.

In another embodiment, the pharmaceutical composition is an eluate from a CEX chromatography using an elution solution with a pH of about 5.1, comprising about 150 mM sodium chloride and an anti-CTLA4 antibody, or antigen binding fragment thereof.

In yet another embodiment, the pharmaceutical composition is an eluate from a CEX chromatography using an elution solution with a pH of about 5.1, comprising about 165 mM sodium chloride and an anti-CTLA4 antibody, or antigen binding fragment thereof.

In still another embodiment, the pharmaceutical composition is an eluate from a CEX chromatography using an elution solution with a pH from about 4.9 to about 5.4 and a conductivity from about 15 mS/cm to about 21 mS/cm and comprising an anti-CTLA4 antibody, or antigen binding fragment thereof.

In one embodiment, the pharmaceutical composition is an eluate from a CEX chromatography using an elution solution with a pH from about 4.9 to about 5.4, comprising from about 135 mM to about 195 mM sodium chloride and an anti-CTLA4 antibody, or antigen binding fragment thereof.

In another embodiment, the pharmaceutical composition is an eluate from a CEX chromatography using an elution solution with a pH from about pH 5.0 to about pH 5.4, comprising from about 150 mM to about 275 mM sodium chloride and an anti-CTLA4 antibody, or antigen binding fragment thereof.

In still another embodiment, the pharmaceutical composition is an eluate from a CEX chromatography using an elution solution with a pH of about 5.1, comprising about 200 mM sodium chloride and an anti-CTLA4 antibody, or antigen binding fragment thereof.

In yet still another embodiment, the pharmaceutical composition is an eluate from a CEX chromatography using an elution solution with a pH of about 5.1, comprising about 250 mM sodium chloride and an anti-CTLA4 antibody, or antigen binding fragment thereof.

In some embodiments of the pharmaceutical compositions, the CEX chromatography is preceded by an AEX chromatography operated in a flowthrough mode.

In certain embodiments of the pharmaceutical compositions, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

In other embodiments of the pharmaceutical compositions, the therapeutic protein is an anti-CTLA4 antibody, or antigen binding fragment thereof.

### Anti-CTLA4 Antibodies and Antigen Binding Fragments Useful in the Invention

Also provided herein is a composition comprising 10-200 mg of an anti-CTLA4 antibody, or antigen binding fragment thereof.

Also provided herein is a composition comprising 10-100 mg of an anti-CTLA4 antibody, or antigen binding fragment thereof.

Also provided herein is a composition comprising an anti-CTLA4 antibody, or antigen binding fragment thereof, selected from the group consisting of: (1) 10 mg of an anti-CTLA4 antibody, or antigen binding fragment thereof; (2) 25 mg of an anti-CTLA4 antibody, or antigen binding fragment thereof; (3) 50 mg of an anti-CTLA4 antibody, or antigen binding fragment thereof; (4) 75 mg of an anti-CTLA4 antibody, or antigen binding fragment thereof; or (5) 100 mg of an anti-CTLA4 antibody, or antigen binding fragment thereof.

Also provided herein is a composition comprising 25 mg of an anti-CTLA4 antibody, or antigen binding fragment thereof.

In one embodiment of the purification methods, treatment methods, compositions, kits and uses of the invention, the anti-CTLA-4 antibody is the human monoclonal antibody 10D1, now known as ipilimumab, and marketed as Yervoy™, which is disclosed in U.S. Pat. No. 6,984,720 and WHO Drug Information 19(4): 61 (2005). In another embodiment, the anti-CTLA-4 antibody is tremelimumab, also known as CP-675,206, which is an IgG2 monoclonal antibody which is described in U.S. Patent Application Publication No. 2012/263677, or PCT International Application Publication Nos. WO 2012/122444 or WO 2007/113648 A2.

In further embodiments of the purification methods, treatment methods, compositions, kits, and uses of the present invention, the anti-CTLA4 antibody, or antigen binding fragment thereof, comprises: light chain CDRs comprising a sequence of amino acids as set forth in SEQ ID NOs: 1, 2 and 3 and heavy chain CDRs comprising a sequence of amino acids as set forth in SEQ ID NOs: 4, 5 and 6.

In other embodiments of the purification methods, treatment methods, compositions, kits, and uses of the present invention, the anti-CTLA4 antibody, is a monoclonal antibody, or antigen binding fragment thereof, which binds to human CTLA4 and comprises (a) a heavy chain variable region comprising an amino acid sequence as set forth in SEQ ID NO: 7 and (b) a light chain variable region comprising an amino acid sequence as set forth in SEQ ID NO: 8.

Exemplary anti-human CTLA4 antibodies
A. Comprises light and heavy chain CDRs
of ipilimumab

| | |
|---|---|
| CDRL1 | RASQSVGSSYLA (SEQ ID NO: 1) |
| CDRL2 | GAFSRAT (SEQ ID NO: 2) |
| CDRL3 | QQYGSSPWT (SEQ ID NO: 3) |
| CDRH1 | SYTMH (SEQ ID NO: 4) |
| CDRH2 | FISYDGNNKYYADSVKG (SEQ ID NO: 5) |
| CDRH3 | TGWLGPFDY (SEQ ID NO: 6) |

B. Comprises the mature heavy chain variable
region and the mature light chain variable
region of ipilimumab

| | |
|---|---|
| Heavy chain VR | QVQLVESGGGVVQPGRSLRLSCAASGFTF |
| | SSYTMHWVRQAPGKGLEWVTFISYDGNNK |
| | YYADSVKGRFTISRDNSKNTLYLQMNSLR |
| | AEDTAIYYCARTGWLGPFDYWGQGTLVTV |
| | SS (SEQ ID NO: 7) |
| Light chain VR | EIVLTQSPGTLSLSPGERATLSCRASQSV |
| | GSSYLAWYQQKPGQAPRLLIYGAFSRATG |
| | IPDRFSGSGSGTDFTLTISRLEPEDFAVY |
| | YCQQYGSSPWTFGQGTKVEIK (SEQ ID |
| | NO: 8) |

C. Comprises the mature heavy chain and
the mature light chain of ipilimumab

| | |
|---|---|
| Heavy chain | SEQ ID NO: 9 |
| Light chain | SEQ ID NO: 10 |

In one embodiment of the purification methods, treatment methods, compositions, kits and uses of the invention, the anti-CTLA-4 antibody is a monoclonal antibody that comprises a heavy chain having the amino acid sequence set forth in SEQ ID NO:9 and a light chain comprising the amino acid sequence set forth in SEQ ID NO:10. In some embodiments, the anti-CTLA4 antibody is an antigen binding fragment of SEQ ID NO:9 and/or SEQ ID NO:10, wherein the antigen binding fragment specifically binds to CTLA4.

In one embodiment of the purification methods, treatment methods, compositions, kits and uses of the invention, the anti-CTLA-4 antibody is any of the anti-CTLA-4 antibodies, or antigen binding fragments thereof, disclosed in International Application Publication No. WO 2016/015675 A1. In one embodiment, the anti-CTLA4 antibody is a monoclonal antibody which comprises the following CDR's:
CDRH1 comprising the amino acid sequence GFTFSDNW (SEQ ID NO:11);
CDRH2 comprising the amino acid sequence IRNKPYNYET (SEQ ID NO:12);
CDRH3 comprising the amino acid sequence TAQFAY (SEQ ID NO:13); and/or
CDRL1 comprising the amino acid sequence ENIYGG (SEQ ID NO:14);
CDRL2 comprising the amino acid sequence GAT (SEQ ID NO:15); and
CDRL3 comprising an amino acid sequence selected from: QNVLRSPFT (SEQ ID NO:16); QNVLSRHPG (SEQ ID NO:17); or QNVLSSRPG (SEQ ID NO:18).

In one embodiment of the purification methods, treatment methods, compositions, kits and uses of the invention, the anti-CTLA4 antibody is 8D2/8D2 (RE) or a variant thereof, 8D2H1L1 or a variant thereof, 8D2H2L2 or a variant thereof, 8D3H3L3 or a variant thereof, 8D2H2L15 or a variant thereof, or 8D2H2117 or a variant thereof.

| Antibody | V_{H} | V_{L} |
|---|---|---|
| 8D2/8D2 | EVKLDETGGGLVQPGRPMKLSCVAS | DIQMTQSPASLSASVGETVTITCGT |
| (RE) | GFTFSDNWMNWVRQSPEKGLEWLA | SENIYGGLNWYQRKQGKSPQLLIF |
| | QIRNKPYNYETYYSDSVKGRFTISRD | GATNLADGMSSRFSGSGSGRQYSL |
| | DSKSSVYLQMNNLRGEDMGIYYCTA | KISSLHPDDVATYYCQNVLRSPFTF |
| | QFAYWGQGTLVTVSA (SEQ ID | GSGTKLEI (SEQ ID NO: 20) |
| | NO: 19) | |
| 8D2H1L1 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKQGKSPKLLIY |
| | QIRNKPYNYETYYSDSVKGRFTISRD | GATNLASGMSSRFSGSGSGTDYTL |
| | DSKNSVYLQMNSLKTEDTGVYYCTA | KISSLHPDDVATYYCQNVLRSPFTF |
| | QFAYWGQGTLVTVSS (SEQ ID | GSGTKLEIK (SEQ ID NO: 22) |
| | NO: 21) | |
| 8D2H2L2 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLRSPFTF |
| | QFAYWGQGTLVTVSS (SEQ ID | GSGTKLEIK (SEQ ID NO: 24) |
| | NO: 23) | |
| 8D2H2L2 | EVQLVESGGGLVQPGGSLRLSCAAS | DIQMTQSPSSLSASVGDRVTITCRT |
| VARIANT 1 | GFTFSDNWMNWVRQAPGKGLEWLA | SENIYGGLNWYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLRSPFTF |
| | QFAYWGQGTLVTVSS (SEQ ID NO: | GSGTKLEIK (SEQ ID NO: 24) |
| | 25) | |
| 8D3H3L3 | EVQLVESGGGLVQPGGSLRLSCAAS | DIQMTQSPSSLSASVGDRVTITCRA |
| | GFTFSDNWMNWVRQAPGKGLEWV | SENIYGGLNWYQQKPGKAPKLLIY |
| | AQIRNKPYNYETEYAASVKGRFTISR | GATSLASGVPSRFSGSGSGTDYTL |
| | DDSKNSAYLQMNSLKTEDTAVYYCT | TISSLQPEDFATYYCQNVLRSPFTF |
| | AQFAYWGQGTLVTVSS (SEQ ID | GSGTKLEIK (SEQ ID NO: 27) |
| | NO: 26) | |
| 8D2H2L15 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLSRHPG |
| | QFAYWGQGTLVTVSS (SEQ ID | FGSGTKLEIK (SEQ ID NO: 29) |
| | NO: 28) | |
| 8D2H2L17 | EVQLVESGGGLVQPGGSMRLSCAAS | DIQMTQSPSSLSASVGDRVTITCRT |
| | GFTFSDNWMNWVRQAPGKGLEWLA | SENIYGGLNWYQRKPGKSPKLLIY |
| | QIRNKPYNYETYYSASVKGRFTISRD | GATNLASGVSSRFSGSGSGTDYTL |
| | DSKNSVYLQMNSLKTEDTGVYYCTA | TISSLQPEDVATYYCQNVLSSRPGF |
| | QFAYWGQGTLVTVSS (SEQ ID | GSGTKLEIK (SEQ ID NO: 31) |
| | NO: 30) | |

In another embodiment of the purification methods, treatment methods, compositions, kits and uses of the invention, the anti-CTLA4 antibody is a variant of 8D2/8D2 (RE), a variant of 8D2H1L1, a variant of 8D2H2L2, a variant of 8D2H2L15, or a variant of 8D2H2L17, wherein the methionine (Met) at position 18 in the VH chain amino acid sequence is independently substituted with an amino acid selected from: Leucine (Leu), Valine (Val), Isoleucine (Ile) or Alanine (Ala). In embodiments of the invention, the anti-CTLA4 antibody comprises the sequence of the 8D2H2L2 Variant 1 as set forth in the table above.

In another embodiment of the purification methods, treatment methods, compositions, kits and uses of the invention, the anti-CTLA4 antibody is 8D2H2L2 Variant 1, having the full heavy chain amino acid sequence set forth in SEQ ID NO: 32 and the full light chain sequence set forth in SEO ID NO: 33.

| Antibody | Full Heavy Chain | Full Light Chain |
|---|---|---|
| 8D2H2L2 | EVQLVESGGGLVQPGGS | DIQMTQSPSSLSASVGD |
| VARIANT 1 | LRLSCAASGFTFSDNWM | RVTITCRTSENIYGGLN |
| | NWVRQAPGKGLEWLAQI | WYQRKPGKSPKLLIYGA |
| | RNKPYNYETYYSASVKG | TNLASGVSSRFSGSGSG |
| | RFTISRDDSKNSVYLQM | TDYTLTISSLQPEDVAT |
| | NSLKTEDTGVYYCTAQF | YYCQNVLRSPFTFGSGT |
| | AYWGQGTLVTVSSASTK | KLEIKRTVAAPSVFIFP |
| | GPSVFPLAPSSKSTSGG | PSDEQLKSGTASVVCLL |
| | TAALGCLVKDYFPEPVT | NNFYPREAKVQWKVDNA |
| | VSWNSGALTSGVHTFPA | LQSGNSQESVTEQDSKD |
| | VLQSSGLYSLSSVVTVP | STYSLSSTLTLSKADYE |
| | SSSLGTQTYICNVNHKP | KHKVYACEVTHQGLSSP |
| | SNTKVDKKVEPKSCDKT | VTKSFNRGEC |
| | HTCPPCPAPELLGGPSV | (SEQ ID NO: 33) |
| | FLFPPKPKDTLMISRTP | |
| | EVTCVVVDVSHEDPEVK | |
| | FNWYVDGVEVHNAKTKP | |
| | REEQYNSTYRVVSVLTV | |
| | LHQDWLNGKEYKCKVSN | |
| | KALPAPIEKTISKAKGQ | |
| | PREPQVYTLPPSRDELT | |
| | KNQVSLTCLVKGFYPSD | |
| | IAVEWESNGQPENNYKT | |
| | TPPVLDSDGSFFLYSKL | |
| | TVDKSRWQQGNVFSCSV | |
| | MHEALHNHYTQKSLSLS | |
| | PGK | |
| | (SEQ ID NO: 32) | |

In one embodiment of the purification methods, treatment methods, compositions, kits and uses of the invention, the anti-CTLA4 antibody is any of the anti-CTLA4 antibodies, or antigen binding fragments thereof, described as disclosed in International Application Publication No. WO 2018/035710 A1, published Mar. 1, 2018.

In another embodiment of the purification methods, treatment methods, compositions, kits and uses of the invention, the anti-CTLA-4 antibody is an antibody, or antigen binding fragment thereof, which cross-competes for binding to human CTLA-4 with, or binds to the same epitope region of human CTLA-4 as does or any of the above described antibodies, including 8D2/8D2 (RE) or variant thereof, 8D2H1L1 or variant thereof, 8D2H2L2 or variant thereof, 8D3H3L3 or variant thereof, 8D2H2L15 or variant thereof, 8D2H2L17.

In an embodiment, a composition comprises; (i) about 10 mg/ml to about 200 mg/ml of an anti-CTLA4 antibody, or antigen binding fragment thereof; about 5 mM to about 20 mM buffer; about 6% to 8% weight/volume (w/v) non-reducing sugar; about 0.01% to about 0.10% non-ionic surfactant; and about 1 mM to about 20 mM anti-oxidant, and wherein the level of PLBL2, LPLA2, and LPL are ≤1 ng/ml of CTLA4 antibody.

In another embodiment, a composition comprises: (i) about 10 mg/ml to about 200 mg/ml of an anti-CTLA4 antibody, or antigen binding fragment thereof; about 5 mM to about 20 mM buffer; about 6% to 8% weight/volume (w/v) non-reducing sugar; about 0.01% to about 0.10% non-ionic surfactant; and about 1 mM to about 20 mM anti-oxidant; a residual amount of host cell lipase, wherein the residual amount of host cell lipase is less than 2 ppm.

In an embodiment, the composition comprises (i) about 10 mg/ml to about 200 mg/ml of an anti-CTLA4 antibody, or antigen binding fragment thereof; (ii) about 5 mM to about 20 mM buffer (iii) about 6% to 8% weight/volume (w/v) non-reducing sugar; (iv) about 0.01% to about 0.10% non-ionic surfactant; and (v) about 1 mM to about 20 mM anti-oxidant, wherein the level of PLBL2, LPLA2, and LPL are ≤1 ng/mg.

In a further embodiment, the non-ionic surfactant is PS-80.

In another embodiment, the composition comprises (i) about 10 mg/ml to about 200 mg/ml of an anti-CTLA4 antibody, or antigen binding fragment thereof; (ii) about 5 mM to about 20 mM buffer (iii) about 6% to 8% weight/volume (w/v) non-reducing sugar; (iv) about 0.01% to about 0.10% polysorbate 80 (PS80); and (v) about 1 mM to about 20 mM anti-oxidant, wherein the level of PLBL2, LPLA2, and LPL are ≤1 ng/mg and the average PS80 degradation is less than or equal to 10% after six months. In another embodiment, the PS-80 degradation refers to the state of PS-80 remaining physically, chemically, and/or biologically stable under common storage conditions.

In another embodiment, the PS-80 degradation can be measured by the amount of intact PS-80 molecules and/or the amount of degraded products using various methods, including but not limited to mass spectrometry (MS), liquid chromatography-mass spectrometry (LCMS), or solid phase extraction (SPE) on a HPLC system with a charged aerosol detector (CAD).

In an embodiment, the composition comprises (i) about 10 mg/ml to about 200 mg/ml of an anti-CTLA4 antibody, or antigen binding fragment thereof; (ii) about 5 mM to about 20 mM buffer (iii) about 6% to 8% weight/volume (w/v) non-reducing sugar; (iv) about 0.01% to about 0.10% non-ionic surfactant; and (v) about 1 mM to about 20 mM anti-oxidant, wherein the level of PLBL2 is ≤1 ng/mg.

In an embodiment, the composition comprises (i) about 10 mg/ml to about 200 mg/ml of an anti-CTLA4 antibody, or antigen binding fragment thereof; (ii) about 5 mM to about 20 mM buffer (iii) about 6% to 8% weight/volume (w/v) non-reducing sugar; (iv) about 0.01% to about 0.10% PS80; and (v) about 1 mM to about 20 mM anti-oxidant, wherein the level of PLBL2 is ≤1 ng/mg and the average PS80 degradation is less than or equal to 10% after six months. In an embodiment, the composition comprises (i) about 10 mg/ml to about 200 mg/ml of an anti-CTLA4 antibody, or antigen binding fragment thereof; (ii) about 5 mM to about 20 mM buffer (iii) about 6% to 8% weight/volume (w/v) non-reducing sugar; (iv) about 0.01% to about 0.10% PS80; and (v) about 1 mM to about 20 mM anti-oxidant, wherein the level of PLBL2 is ≤1 ng/mg and the average PS80 degradation is equal to or less than 10% after six months, and wherein the PS80 degradation is measured by the amount of intact PS-80 molecules and/or the amount of degraded products using various methods, including but not limited to mass spectrometry (MS), liquid chromatography-mass spectrometry (LCMS), or solid phase extraction (SPE) on a HPLC system with a charged aerosol detector (CAD).

In an embodiment, the composition comprises (i) about 50 mg/ml of an anti-CTLA4 antibody, or antigen binding fragment thereof; (ii) about 10 mM buffer (iii) about 7% weight/volume (w/v) non-reducing sugar; (iv) about 0.02% non-ionic surfactant; and (v) about 1 mM to about 20 mM anti-oxidant, wherein the level of PLBL2 is ≤1 ng/mg.

In an embodiment, the composition comprises (i) about 50 mg/ml of an anti-CTLA4 antibody, or antigen binding fragment thereof (ii) about 10 mM buffer (iii) about 7% weight/volume (w/v) non-reducing sugar; (iv) about 0.02% PS80; and (v) about 1 mM to about 20 mM anti-oxidant, wherein the level of PLBL2 is ≤1 ng/mg and the average PS80 degradation is less than or equal to 10% after six months.

In one embodiment, the formulation has a pH between 4.5-6.5. In particular embodiments, the pH of the formulation is from about pH 5.0 to about pH 6.0. In a further embodiment, the pH of the formulation is from about pH 5.3 to about pH 5.8. In another embodiment, the pH is 5.3. In another embodiment, the pH is 5.4. In one embodiment, the pH is 5.5. In one embodiment, the pH is 5.6. In a further embodiment, the pH is 5.7. In an embodiment, the pH is 5.8.

In one embodiment of the formulation, the buffer is L-histidine buffer or sodium acetate buffer, the non-reducing sugar is sucrose, the non-ionic surfactant is polysorbate 80, and the antioxidant is methionine, or a pharmaceutically acceptable salt thereof. In one embodiment, the anti-oxidant is L-methionine. In another embodiment, the anti-oxidant is a pharmaceutically acceptable salt of L-methionine, such as, for example, methionine HCl.

In one embodiment, the PS-80 concentration in the composition remains±0.02 mg/mL as compared to the time zero result.

In another embodiments, the composition according to any of the above-described methods, wherein the composition is administered to a patient.

### IV. BRIEF DESCRIPTION OF THE DRAWINGS

FIGS. 1A-1F show PLBL2 (FIGS. 1A-1C) or LPLA2 (FIGS. 1D-1F) log K_{P} values for a range of typical AEX conditions. FIG. 1A and FIG. 1D show conditions typical for loading in Tris and acetate buffer. FIG. 1B and FIG. 1E show conditions typical for equilibrating or washing with Tris buffer. FIG. 1C and FIG. 1F show conditions for equilibrating or washing with phosphate buffer.

FIGS. 2A-2C show PLBL2 (FIGS. 2A and 2B) or LPLA2 (FIG. 2C) log K_{P} values for a range of typical CEX conditions. FIG. 2A and FIG. 2C show conditions for modulation of binding by mainly varying salt. FIG. 2B shows conditions for modulation of binding by mainly varying pH.

FIG. 3 shows PLBL2 or LPLA2 log K_{P} values for a range of HIC conditions typical for modulation of binding by salt concentration.

FIGS. 4A and 4B show PLBL2 log K_{P} values for a range of pH and salt conditions typical for multimodal chromatography resins. FIG. 4A shows conditions for a multimodal anion exchanger, Capto adhere. FIG. 4B shows conditions for a multimodal cation exchanger, Capto MMC.

FIG. 5 shows the chromatogram of an AEX process operated in flowthrough mode under the specified condition, indicating that very little mAb3 bound to the AEX resin.

FIGS. 6A-6C show log α separation factor values for mAb3 and PLBL2 (FIGS. 6A and 6B) or mAb3 and LPLA2 (FIG. 6C) at a range of pH and salt conditions typical for CEX chromatography. FIGS. 6A and 6C demonstrate optimizing separation conditions where binding is modulated by mainly varying salt concentrations. FIG. 6B demonstrates optimizing conditions where binding is modulated by mainly varying pH. Black boxes represent regions where separation is maximized.

FIG. 7 shows a comparison of log K_{P} values on a HIC resin for PLBL2, LPLA2, and two different mAbs, mAb2 and mAb3. mAb3 has very similar binding to PLBL2 and LPLA2, but mAb2 is bound weaker than mAb3, PLBL2, and LPLA2, offering greater separation potential of PLBL2 and LPLA2 from mAb2 than from mAb3.

FIGS. 8A and 8B show log α values for mAb3 and PLBL2 at typical operating conditions for multimodal chromatography resins. FIG. 8A demonstrates optimizing separation conditions for a multimodal anion exchanger, Capto adhere. FIG. 8B demonstrates optimizing separation conditions for a multimodal cation exchanger, Capto MMC. Black boxes represent regions where separation is maximized.

FIG. 9 shows Pareto chart summarizing the ranked statistical significance of model parameters (factors) for residual HCP (ng/mg).

FIGS. 10A-10C show PS-80 concentration of the placebo, mAb4 Anion Exchange Chromatography (AEX) pool drug substance (AEXP DS), and mAb4 Cation Exchange Chromatography (CEX) pool drug substance (CEXP DS) at 5° C. (FIG. 10A), 25° C. (FIG. 10B), and 40° C. (FIG. 10C) over 26 weeks.

FIGS. 11A and 11B show percentage of PS80 degradation in formulations containing mAb4 purified by two-column chromatography (Protein A and AEX, FIG. 11A) or three-column chromatography (Protein A, AEX, and CEX, FIG. 11B).

FIG. 12 demonstrates that the three-column chromatography is necessary to fully remove residual lipases for mAb4.

FIG. 13 shows a PS-80 stability study that was conducted on the combined CEXP runs at 5±3° C. and 25±3° C. for 24 weeks and 16 weeks.

FIG. 14 shows PLBL2 spiking results for AEX

FIG. 15 shows PLBL2 spiking results for CEX

FIG. 16 shows LPL spiking results for AEX

FIG. 17 shows LPL spiking results for CEX

FIG. 18 shows basic variants spiking results for CEX

FIG. 19 shows total high molecular weight species spiking results for CEX. The total HMW capacity for CEX was approximately 10.8%.

### V. DETAILED DESCRIPTION OF THE INVENTION

### 1. Definitions

Certain technical and scientific terms are specifically defined below. Unless specifically defined elsewhere in this document, all other technical and scientific terms used herein have the meaning commonly understood by one of ordinary skill in the art to which this disclosure relates. In case of conflict, the present specification, including definitions, will control.

The terms “operating condition,” “operation condition,” “processing condition,” or “process condition,” exchangeable and as used herein, refer to the condition for operating a chromatographic process. The operating condition can be equilibration condition, loading condition, wash condition, and/or elution condition, etc. The operating condition includes but is not limited to the type of the chromatographic resin, the resin backbone, the resin ligand, the pH of the operating solution, the composition of the operating solution, the concentration of each ingredient of the operating solution, the conductivity of the operating solution, the ionic strength of the operating solution, the cationic strength of the operating solution, the anionic strength of the operating solution, or a combination of two or more above factors.

The term “operating solution” refers to the solution used in operating a chromatographic process. The operating solution can be equilibration solution, loading or feed solution, wash solution, and/or elution solution, etc.

The term “partition coefficient” or “K_{P},” as used herein, refers to the ratio of the concentration of a protein bound to a chromatographic resin (Q) to the concentration of the protein remaining in the solution (C) at equilibrium under a specific operating condition. The partition coefficient for a particular protein can be calculated as follows: K_{P}=Q/C.

The term “separation factor” or “α,” as used herein, refers to the ratio of the partition coefficient for a first protein (K_{P, protein 1}) and the partition coefficient for a second protein (K_{P, protein 2}). The separation factor quantifies the selectivity of a chromatographic resin between the two proteins, under a specific operating condition. It can be used to predict the extent of separation of the two proteins through the chromatographic resin under the operating condition. The separation factor between two proteins can be calculated as follows: α=K_{P, protein 1}/K_{P, protein 2}; or log α=log K_{P, protein 1}−log K_{P, protein 2}.

“Production protein,” as used herein, refers to any protein that is the intended product of a bioprocess. Non-limiting examples of the production protein include therapeutic proteins, antibodies (e.g., monoclonal antibodies bispecific antibodies, or antigen-binding fragments thereof, etc), hormones, cytokines, enzymes, growth factors, clotting factors, or immunoconjugates thereof, fusion proteins thereof, or fragments thereof. One example of a production protein includes, but is not limited to, an anti-CTLA4 antibody.

“Therapeutic protein,” as used herein, refers to any protein that has therapeutic effect in an animal (e.g., human, cow, horse, dog, etc.). Non-limiting examples of the therapeutic protein include antibodies (e.g., monoclonal antibodies, bispecific antibodies, or antigen-binding fragments thereof, etc.), hormones, cytokines, enzymes, growth factors, dotting factors, or immunoconjugates thereof, fusion proteins thereof, or fragments thereof.

“Lipase”, as used herein, generally refers to host cell lipases and related proteins/enzymes, including (for the Chinese Hamster Ovary (CHO) expression system: PLBL2, LPL, LPLA2, LP-PLA2, and LAL.

“Eluate,” as used herein, refers to the liquid that passes through a chromatography. In some embodiments, the eluate is the flowthrough of a loading solution. In other embodiments, the eluate comprises the elution solution that passes through the chromatography and any additional components eluted from the chromatography.

“Mixed mode” or “multimodal,” when used with a chromatographic resin, means that the resin can separate molecules by more than one mode, function, or mechanism, for example, an ion exchange and a hydrophobic interaction. In some embodiments, the mixed mode or multimodal chromatographic resin can separate molecules by both cation exchange and hydrophobic interaction. In other embodiments, the mixed mode or multimodal chromatographic resin can separate molecules by both anion exchange and hydrophobic interaction.

“Polysorbate-80 stability” or “PS-80 stability,” as used herein, refers to the state of PS-80 remaining physically, chemically, and/or biologically stable under common storage conditions (e.g., 5° C.±3° C., 25° C.±3° C., 60%±5% relative humidity (RH), 40° C.±2° C., 75%±5% relative humidity (RH)) over a period of time (e.g., 1 week, 1 month, 6 months, 1 year, 2 years, etc.) The PS-80 stability can be measured by the amount of intact PS-80 molecules and/or the amount of degraded products using various methods, including but not limited to mass spectrometry (MS), liquid chromatography-mass spectrometry (LCMS), or solid phase extraction (SPE) on a HPLC system with a charged aerosol detector (CAD).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same. Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5ₜₕ ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

An “anti-CTLA-4 antibody” means an antibody, or antigen binding fragment thereof, which binds to human CTLA-4 so as to disrupt the interaction of CTLA-4 with a human B7 receptor. After binding to B7, CTLA4 can inhibit the activation of mouse and human T cells, playing a negative regulating role in the activation of T cells. As used herein, unless specifically stated, said B7 refers to B7-1 and/or B7-2; and their specific protein sequences refer to the sequences known in the art. Reference can be made to the sequences disclosed in the literature or GenBank, e.g., B7-1 (CD80, NCBI Gene ID: 941), B7-2 (CD86, NCBI Gene ID: 942). An anti-CTLA4 antibody may be a human antibody, a humanized antibody or a chimeric antibody, and may include a human constant region. In some embodiments the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and FIT fragments.

“CDR” or “CDRs” means complementarity determining region(s) in a immunoglobulin variable region, defined using the Kabat numbering system, unless otherwise indicated.

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“About” when used to modify a numerically defined parameter (e.g., pH, concentration, etc.) means that the parameter is within 20%, within 15%, within 10%, within 9%, within 8%, within 7%, within 6%, within 5%, within 4%, within 3%, within 2%, within 1%, or less of the stated numerical value or range for that parameter; where appropriate, the stated parameter may be rounded to the nearest whole number.

As used herein, including the appended claims, the singular forms of words such as “a,” “an,” and “the,” include their corresponding plural references unless the context clearly dictates otherwise. Unless otherwise required by context, singular terms shall include pluralities and plural terms shall include the singular.

As used herein, the terms “at least one” item or “one or more” item each include a single item selected from the list as well as mixtures of two or more items selected from the list.

Any example(s) following the term “e.g.” or “for example” is not meant to be exhaustive or limiting.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. All ranges also are intended to include all included sub-ranges, although not necessarily explicitly set forth. For example, a range of pH 4.0-5.0 is intended to include pH 4.0, 4.1, 4.13, 4.2, 4.1-4.6, 4.3-4.4, and 5.0. In addition, the term “or,” as used herein, denotes alternatives that may, where appropriate, be combined; that is, the term “or” includes each listed alternative separately as well as their combination.

The term “patient” (alternatively referred to as “subject” or “individual” herein) refers to a mammal (e.g., rat, mouse, dog, cat, rabbit) capable of being treated with the compositions of the invention, most preferably a human. In some embodiments, the patient is an adult patient. In other embodiments, the patient is a pediatric patient.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Exemplary methods and materials are described herein, although methods and materials similar or equivalent to those described herein can also be used in the practice or testing of the present disclosure. The materials, methods, and examples are illustrative only and not intended to be limiting.

### 2. Chromatographic Processes

The various methods provided herein can be used with any chromatographic process disclosed herein or understood by a person of ordinary skill in the art, for separation of a an anti-CTLA4 antibody, or antigen binding fragment thereof, from impurities. Non-limiting examples of chromatographic processes include IEX, AEX, CEX, HIC, mixed mode AEX, mixed mode CEX, affinity, and hydroxyapatite chromatographic (HAC) process, etc. In one embodiment, the chromatographic process is an IEX chromatographic process. In another embodiment, the chromatographic process is an AEX chromatographic process. In yet another embodiment, the chromatographic process is a CEX chromatographic process. In still another embodiment, the chromatographic process is a HIC chromatographic process. In one embodiment, the chromatographic process is a mixed mode IEX chromatographic process. In another embodiment, the chromatographic process is a mixed mode AEX chromatographic process. In yet another embodiment, the chromatographic process is a mixed mode CEX chromatographic process. In still another embodiment, the chromatographic process is an affinity chromatographic process. In one embodiment, the chromatographic process is a protein A chromatographic process. In another embodiment, the chromatographic process is a protein G affinity chromatographic process. In yet another embodiment, the chromatographic process is an immobilized metal affinity chromatographic (IMAC) process. In still another embodiment, the chromatographic process is a HAC process.

IEX chromatography separates molecules based on net charge of the molecules. Separation occurs as a result of competition between the charged molecule of interest and counter ions for oppositely charged ligand groups on the IEX chromatographic resin. Strength of the binding of the molecule to the IEX resin depends on the net charge of the molecules, which is affected by operating conditions, such as pH and ionic strength. IEX resins include AEX resins and CEX resins. AEX resins may contain substituents such as diethylaminoethyl (DEAE), trimethyalaminoethyl (TMAE), quaternary aminoethyl (QAE) and quaternary amine (QA) groups. CEX resins may contain substituents such as carboxymethyl (CM), sulfoethyl (SE), sulfopropyl (SP), phosphate (P) and sulfonate (S). Cellulosic IEX resins such as DE23, DE32, DE52, CM-23, CM-32 and CM-52 are available from Whatman Ltd. Maidstone, Kent, U.K. Sephadex-based and cross-linked IEX resins are also known. For example, DEAE-, QAE-, CM-, and SP-Sephadex, and DEAE-, Q-, CM- and S-Sepharose, and Sepharose are all available from GE Healthcare, Piscataway, NJ. Further, both DEAE and CM derived ethylene glycol-methacrylate copolymer such as TOYOPEARL™ DEAE-650S or M and TOYOPEARL™ CM-650S or M are available from Toso Haas Co., Philadelphia, PA. POROS™ HS, POROS™ HQ, POROS™ XS are available from Thermo Fisher Scientific, Waltham, MA.

HIC chromatography separates molecules based on hydrophobicity of molecules. Hydrophobic regions in the molecule of interest bind to the HIC resin through hydrophobic interaction. Strength of the interaction depends on operating conditions such as pH, ionic strength, and salt concentration. In general, HIC resins contain a base matrix (e.g., cross-linked agarose or synthetic copolymer material) to which hydrophobic ligands (e.g., alkyl or aryl groups) are coupled. Non-limiting examples of HIC resins include Phenyl SEPHAROSE™ 6 FAST FLOW™ (Pharmacia LKB Biotechnology, AB, Sweden); Phenyl SEPHAROSE™ High Performance (Pharmacia LKB Biotechnology, AB, Sweden); Octyl SEPHAROSE™ High Performance (Pharmacia LKB Biotechnology, AB, Sweden); Fractogel™ EMD Propyl or FRACTOGEL™ EMD Phenyl (E. Merck, Germany); MACRO-PREP™ Methyl or MACRO-PREP™ t-Butyl Supports (Bio-Rad, CA); WP HI-Propyl (C₃)™ (J. T. Baker, NJ); TOYOPEARL™ ether, phenyl or butyl (TosoHaas, PA); and Tosoh-Butyl-650M (Tosoh Corp., Tokyo, Japan).

HAC chromatography uses an insoluble hydroxylated calcium phosphate of the formula [Ca₁₀(PO₄)₆(OH)₂] as both the matrix and the ligand. The functional groups of the HAC resin include pairs of positively charged calcium ions (C-sites) and negatively charged phosphate groups (P-sites). The C-sites can interact with carboxylate residues on the protein surface while the P-sites can interact with basic protein residues. Strength of the binding between the protein and the HAC resin depends on operating conditions including pH, ionic strength, composition of solution, concentration of each component of the composition, gradient of pH, gradient of component concentration, etc. Various HAC resins, such as CHT™ Ceramic Hydroxyapatite and CFT™ Ceramic Fluoroapatite, are commercially available.

Affinity chromatography separates molecules based on a highly specific interaction between the molecule of interest and the functional group of the resin, such as interaction between antigen and antibody, enzyme and substrate, receptor and ligand, or protein and nucleic acid, etc. Some commonly used affinity chromatographic resins include protein A or protein G resin to purify antibodies, avidin biotin resin to purify biotin/avidin and their derivatives, glutathione resin to purify GST-tagged recombinant proteins, heparin resin to separate plasma coagulation proteins, IMAC resin to purify proteins that specifically interact with the metal ions, etc. Operating conditions of each affinity chromatography depend on the mechanism of the interaction and factors that affect the interaction. Commercial affinity chromatographic resins include but are not limited to MabSelect Sure, UNOsphere SUPrA™, Affi-Gel®, and Affi-Prep®.

In certain embodiments, the chromatographic resin employed herein can separate molecules based on more than one function or mechanism, i.e., in a mixed mode. The mixed mode can be a combination of any two or more functions or mechanisms described above or understood by a person of ordinary skill in the art, such as a combination of IEX and HIC (e.g., AEX/HIC or CEX/HIC), a combination of AEX and CEX (AEX/CEX), or a combination of HIC, AEX, and CEX (HIC/AEX/CEX), etc. Exemplary mixed mode chromatographic resins include but are not limited to OminPac PCX-500, Primesep, Obelisc R, Oblisc N, Acclaim Trinity P1, Acclaim Trinity P2, Capto Adhere, Capto Adhere Impres, Capto MMC, Capto MMC Impres, Capto Core 700, PPA Hypercel, HEA Hypercel, MEP Hypercel, Eshmuno HCX, Toyopearl MX-Trp-650M, Nuvia C Prime, CHT Type I, and CHT Type II.

### 3. Partition Coefficient (K_{P}) and Separation Factor (α)

Partition coefficient (K_{P}) and separation factor (α) are two thermodynamic parameters specific for an operating condition of a chromatographic process, which can be used to quantify separation that can be achieved through the process under the operating condition.

A partitioning coefficient, K_{P}, is determined by mixing a known liquid concentration of protein (or other molecule of interest) with a known volume of chromatographic resin and calculating the ratio of the protein bound to the resin and the protein remaining in the liquid at equilibrium: K_{P}=q/c=[bound]/[free].

Partitioning is generally reported in terms of log K_{P}, which can be accurately quantified from approximately 0 to 2 using the UV method described herein. General rules for log K_{P} screening are as follows:
log K_{P}≥1.5, strong binding to the resin;
log K_{P}<1, conditions where elution would be expected for a bind-and-elute modality;
0.5<log K_{P}<1, weak interaction conditions that will show some binding;
log K_{P}<0.5, very little or no binding.

The difference of log K_{P} values between different species can be used to predict separation of the species through the calculation of a separation factor, α, as follows: α=K_{P, protein 1}/K_{P, protein 2}; log α=log K_{P, protein 1}−log K_{P, protein 2}, where a log α further from 0 indicates better separation. In certain embodiments, an absolute value of log α larger than 0.2 indicates good separation between the two species. In some embodiments, an absolute value of log α larger than 0.3 indicates good separation between the two species. In other embodiments, an absolute value of log α larger than 0.5 indicates good separation between the two species. In other embodiments, an absolute value of log α larger than 1.0 indicates good separation between the two species.

### 4. HCP and Production Proteins

The various methods provided herein apply to a broad variety of HCP as well as a broad variety of production proteins.

The HCP can be any endogenous protein derived from a host cell (e.g., CHO cell) during bioprocessing of a production protein expressed in the host cell. Non-limiting examples of HCP include structural protein, functional protein, secreted protein, enzyme, such as lipase, proteinase, and kinase, etc. In some embodiments, the HCP is a structural protein. In certain embodiments, the HCP is a functional protein. In other embodiments, the HCP is a secreted protein. In yet other embodiment, the HCP is an enzyme. In one embodiment, the HCP is a lipase. In another embodiment, the HCP is a proteinase. In yet another embodiment, the HCP is a kinase.

In certain embodiments, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

The host cell can be any cell used for expressing an exogenous protein. Common host cells used in manufacturing of biopharmaceuticals include but are not limited to CHO cell, baby hamster kidney (BHK21) cell, murine myeloma NS0 cell, murine myeloma Sp2/0 cell, human embryonic kidney 293 (HEK293) cell, fibrosarcoma HT-1080 cell, PER.C6 cell, HKB-11 cell, CAP cell, HuH-7 cell, murine C127 cell, and a naturally generated or genetically modified variant thereof. In certain embodiments, the host cell is CHO cell. In some embodiments, the host cell is baby hamster kidney (BHK21) cell. In other embodiments, the host cell is murine myeloma NS0 cell. In yet other embodiments, the host cell is murine myeloma Sp2/0 cell. In still other embodiments, the host cell is human embryonic kidney 293 (HEK293) cell. In certain embodiments, the host cell is fibrosarcoma HT-1080 cell. In some embodiments, the host cell is PER.C6 cell. In other embodiments, the host cell is HKB-11 cell. In yet other embodiments, the host cell is CAP cell. In still other embodiments, the host cell is HuH-7 cell. In certain embodiments, the host cell is murine C127 cell. In some embodiments, the host cell is a naturally generated variant of the above host cell. In other embodiments, the host cell is a genetically modified variant of the above host cell.

In certain embodiments, the CHO cell lipase is selected from the group consisting of PLBL2, LPL, LPLA2, LP-PLA2, and LAL. In one embodiment, the CHO cell lipase is PLBL2. In another embodiment, the CHO cell lipase is LPL. In yet another embodiment, the CHO cell lipase is LPLA2. In one embodiment, the CHO cell lipase is LP-PLA2. In another embodiment, the CHO cell lipase is LAL. In still another embodiment, the CHO cell lipase includes two, three, four, five, six, seven, eight, nine, ten, or more different CHO cell lipases. In yet still another embodiment, the CHO cell lipase includes two, three, four, or five different CHO cell lipases selected from the group consisting of PLBL2, LPL, LPLA2, LP-PLA2, and LAL. In one embodiment, the CHO cell lipase includes PLBL2 and LPL. In another embodiment, the CHO cell lipase includes PLBL2 and LPLA2. In yet another embodiment, the CHO cell lipase includes PLBL2 and LP-PLA2. In still another embodiment, the CHO cell lipase includes PLBL2 and LAL. In one embodiment, the CHO cell lipase includes LPL and LPLA2. In another embodiment, the CHO cell lipase includes LPL and LP-PLA2. In yet another embodiment, the CHO cell lipase includes LPL and LAL. In still another embodiment, the CHO cell lipase includes LPLA2 and LP-PLA2. In one embodiment, the CHO cell lipase includes LPLA2 and LAL. In another embodiment, the CHO cell lipase includes LP-PLA2 and LAL. In yet another embodiment, the CHO cell lipase includes PLBL2, LPL, and LPLA2. In still another embodiment, the CHO cell lipase includes PLBL2, LPL, and LP-PLA2. In one embodiment, the CHO cell lipase includes PLBL2, LPL, and LAL. In another embodiment, the CHO cell lipase includes PLBL2, LPLA2, and LP-PLA2. In yet another embodiment, the CHO cell lipase includes PLBL2, LPLA2, and LAL. In still another embodiment, the CHO cell lipase includes PLBL2, LP-PLA2, and LAL. In one embodiment, the CHO cell lipase includes LPL, LPLA2, and LP-PLA2. In another embodiment, the CHO cell lipase includes LPL, LPLA2, and LAL. In yet another embodiment, the CHO cell lipase includes LPL, LP-PLA2, and LAL. In still another embodiment, the CHO cell lipase includes LPLA2, LP-PLA2, and LAL. In one embodiment, the CHO cell lipase includes PLBL2, LPL, LPLA2, and LP-PLA2. In another embodiment, the CHO cell lipase includes PLBL2, LPL, LPLA2, and LAL. In yet another embodiment, the CHO cell lipase includes PLBL2, LPL, LP-PLA2, and LAL. In still another embodiment, the CHO cell lipase includes PLBL2, LPLA2, LP-PLA2, and LAL. In yet still another embodiment, the CHO cell lipase includes PLBL2, LPL, LPLA2, LP-PLA2, and LAL.

The production protein can be any protein of interest expressed in the host cell for the purpose of generating a biopharmaceutical product. Non-limiting examples of production proteins include therapeutic proteins, monoclonal antibodies, hormones, cytokines, growth factors, clotting factors, enzymes, fusion proteins thereof, immunoconjugates thereof, and fragments thereof. In certain embodiments, the production protein is a therapeutic protein. In some embodiments, the production protein is a monoclonal antibody. In other embodiments, the production protein is a hormone. In yet other embodiments, the production protein is a cytokine. In still other embodiments, the production protein is a growth factor. In certain embodiments, the production protein is a clotting factor. In some embodiments, the production protein is an enzyme. In other embodiments, the production protein is a fusion protein of the above production protein. In yet other embodiments, the production protein is an immunoconjugate of the above production protein. In still other embodiments, the production protein is a fragment of the above production protein.

In some embodiments, the production protein is a monoclonal antibody specific for an antigen including but not limited to PD-1, PD-L1, CTLA-4, LAG3, TIM3, TIGIT, GITR, TNF-α, HER2, GPIIb/IIIa, CD52, PCSK9, IL-2Rα, BLyS, VEGF, Clostridium difficile toxin B, CD19, CD30, IL-1β, IL17Rα, PSMA, EGFR, IL-2R, IL-2Rβγ, CD38, RANKL, GD2, IL-4Rα, complement component 5, CD20, SLAMF7, dabigatran, IL-5, α-4 integrin, PDGFRα, VEGFR1, VEGFR2, F protein of RSV, IL-6, IL-6R, IL-12, IL-23, and CD33. In one embodiment, the production protein is an anti-PD-1 monoclonal antibody. In another embodiment, the production protein is an anti-CTLA-4 monoclonal antibody. In another embodiment, the production protein is an anti-CTLA-4 monoclonal antibody or antigen binding fragment thereof. In yet another embodiment, the production protein is an anti-LAG3 monoclonal antibody. In still another embodiment, the production protein is an anti-TIGIT monoclonal antibody. In one embodiment, the production protein is an anti-GITR monoclonal antibody.

In a specific embodiment, the anti-PD-1 monoclonal antibody is pembrolizumab. In another embodiment, the anti-PD-1 monoclonal antibody is nivolumab. In yet another embodiment, the anti-PD-1 monoclonal antibody is pidilizumab (U.S. Pat. No. 7,332,582). In still another embodiment, the anti-PD-1 monoclonal antibody is AMP-514 (MedImmune LLC, Gaithersburg, MD). In another embodiment, the anti-PD-1 monoclonal antibody is PDR001 (U.S. Pat. No. 9,683,048). In yet another embodiment, the anti-PD-1 monoclonal antibody is BGB-A317 (U.S. Pat. No. 8,735,553). In still another embodiment, the anti-PD-1 monoclonal antibody is MGA012 (MacroGenics, Rockville, MD).

In one embodiment, the anti-LAG3 monoclonal antibody is BMS-986016 (Bristol-Myers Squibb, New York, NY). In another embodiment, the anti-LAG3 monoclonal antibody is REGN3767 (Regeneron, Tarrytown, NY). In yet another embodiment, the anti-LAG3 monoclonal antibody is LAG525 (Novartis, Basel, Switzerland). In still another embodiment, the anti-LAG3 monoclonal antibody is GSK2813781 (GlaxoSmithKline, Brentford, UK).

In one embodiment, the anti-TIGIT monoclonal antibody is BMS-986207 (Bristol-Myers Squibb, New York, NY). In another embodiment, the anti-TIGIT monoclonal antibody is OMP-313M32 (OncoMed Pharmaceuticals, Redwood city, CA). In yet another embodiment, the anti-TIGIT monoclonal antibody is MTIG7192A (also known as RG6058, U.S. Publ. No. 2017/0088613). In still another embodiment, the anti-TIGIT monoclonal antibody is PTZ-201 (Potenza Therapeutics, Cambridge, MA; also known as ASP8374, Astellas Pharma, Tokyo, Japan).

### 5. Methods of Screening Operating Conditions for Separation of a Host Cell Lipase from a Production Protein

This disclosure provides methods of screening operating conditions for separation of a HCP (e.g., lipase) from a production protein (e.g., monoclonal antibody) through a chromatographic process.

Screening can be done by batch binding studies, mini-column binding studies, or any other methods that one of ordinary skill in the art would understand. A plethora of combinations of chromatographic resins and operating conditions, including pH, with or without salt, salt type, salt concentration, other components (e.g., counter ion) in solution, concentration of each component, or load protein concentration, etc., can be designed and examined for the HCP (e.g., lipase) or the production protein (e.g., monoclonal antibody). The K_{P} values of the HCP (e.g., lipase) and the production protein (e.g., monoclonal antibody) are determined by methods disclosed herein or commonly understood by a person of ordinary skill in the art. Log α values between the HCP (e.g., lipase) and the production protein (e.g., monoclonal antibody) are calculated using methods described herein. In general, an absolute value of log α larger than 0.5 is desirable for good separation between the HCP (e.g., lipase) and the production protein (e.g., monoclonal antibody).

Chromatographic resins to be screened can be any chromatographic resins that may separate the HCP (e.g., lipase) from the production protein (e.g., monoclonal antibody) based on characteristics of the HCP (e.g., lipase) and the production protein (e.g., monoclonal antibody). Operating conditions to be screened can be commonly used process conditions for each resin selected, for example, equilibration condition, loading condition, washing condition, elution condition, or stripping condition, etc.

In one embodiment, the screening is performed using a resin slurry plate method, as disclosed in Welsh et al., Biotechnol Prog. 30 (3):626-635 (2014). For example, mixtures of different combinations of pH, salt, and feed are added into 96-well filter plates (e.g., P/N MSBVN1250, Millipore Sigma, Burlington, MA). The chromatographic resin volume is 2-50 μL, and the liquid feed volume is 200 μL. In some embodiments, 16-32 conditions are tested for each resin. In other embodiments, 24-96 conditions are tested for each resin. Separation of resin and liquid is accomplished by vacuum filtration. First, the resin is incubated with the equilibration buffer for 10 minutes and the equilibration step is repeated three times. Next, the resin is incubated with feed for 60 minutes. Then, the resin is incubated in strip condition for 10 minutes and repeated twice. The equilibration step allows for buffer exchange from the initial resin slurry buffer. The 60 min time for feed mixing allows for pseudo equilibration between the resin ligand and protein at a given set of conditions. The filtrate from the feed step was measured by UV absorbance at 280-320 nm to determine the final liquid concentration of the protein, c. The bound concentration of the protein, q, is determined by a mass balance of c and the known feed concentration, c₀.

In another embodiment, the screening is performed using a mini-column method, as disclosed in Welsh et al., Biotechnol Prog. 30 (3):626-635 (2014) or Petroff et al., Biotech Bioeng. 113 (6):1273-1283 (2015). For example, mixtures of different combinations of pH, salt, and feed are screened in a 0.6 mL column format with a 3 cm bed height. Up to 8 columns are screened in parallel. A typical residence time of about 4 min is preserved in the miniature columns by reducing the linear flowrate from about 300 cm/h for a typical column to about 45 cm/h in the miniature column format. All other typical parameters for chromatography screening are conserved. Eluate factions can be collected as pools or as fractions by collecting in 96-well plates to produce chromatograms similar to lab scale studies.

Once the operating conditions for separating the HCP (e.g., lipase) from the production protein (e.g., monoclonal antibody) are determined, the conditions of the load fluid and/or resin can be adjusted accordingly. For example, the resin can be equilibrated by washing it with a solution that will bring it to the necessary operating conditions.

### 6. Methods of Separating a Host Cell Lipase from a Production Protein

This disclosure further provides methods of separating an HCP (e.g., lipase) from a production protein (e.g., monoclonal antibody) through a chromatographic process.

In one aspect, provided herein is a method of separating a host cell lipase from a anti-CTLA4 antibody, or antigen binding fragment thereof, through a chromatographic process, comprising:
(a) passing a load fluid comprising the lipase and the anti-CTLA4 antibody, or antigen binding fragment thereof, through a chromatographic resin under a loading operating condition; and
(b) collecting the anti-CTLA4 antibody, or antigen binding fragment thereof, in a flowthrough;
   wherein separation factor (α) is the ratio of the partition coefficient (K_{P}) for the lipase to the K_{P} for the anti-CTLA4 antibody, or antigen binding fragment thereof, and wherein log α is larger than 0.5 under the loading operating condition.

In certain embodiments, log α is larger than 1.0 under the loading operating condition.

In some embodiments, the log K_{P} for the lipase is larger than 1.0 under the loading operating condition. In other embodiments, the log K_{P} for the lipase is larger than 1.5 under the loading operating condition.

In certain embodiments, log α is larger than 0.5 and the log K_{P} for the lipase is larger than 1.0 under the loading operating condition. In some embodiments, log α is larger than 0.5 and the log K_{P} for the lipase is larger than 1.5 under the loading operating condition. In other embodiments, log α is larger than 1.0 and the log K_{P} for the lipase is larger than 1.0 under the loading operating condition. In yet other embodiments, log α is larger than 1.0 and the log K_{P} for the lipase is larger than 1.5 under the loading operating condition.

In another aspect, provided herein is a method of separating a host cell lipase from an anti-CTLA4 antibody, or antigen binding fragment thereof, through a chromatographic process, comprising:
(a) passing a load fluid comprising the lipase and the anti-CTLA4 antibody, or antigen binding fragment thereof, through a chromatographic resin; and
(b) eluting the anti-CTLA4 antibody, or antigen binding fragment thereof, from the chromatographic resin with an elution solution under an elution operating condition;
   wherein α is the ratio of K_{P} for the lipase to the K_{P} for the anti-CTLA4 antibody, or antigen binding fragment thereof, and wherein log α is larger than 0.5 under the elution operating condition.

In certain embodiments, log α is larger than 1.0 under the elution operating condition.

In some embodiments, the log K_{P} for the lipase is larger than 1.0 under the elution operating condition. In other embodiments, the log K_{P} for the lipase is larger than 1.5 under the elution operating condition.

In certain embodiments, log α is larger than 0.5 and the log K_{P} for the lipase is larger than 1.0 under the elution operating condition. In some embodiments, log α is larger than 0.5 and the log K_{P} for the lipase is larger than 1.5 under the elution operating condition. In other embodiments, log α is larger than 1.0 and the log K_{P} for the lipase is larger than 1.0 under the elution operating condition. In yet other embodiments, log α is larger than 1.0 and the log K_{P} for the lipase is larger than 1.5 under the elution operating condition.

In some embodiments of various methods provided herein, the lipase is a CHO cell lipase.

In certain embodiments, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

In certain embodiments, the CHO cell lipase is selected from the group consisting of PLBL2, LPL, LPLA2, LP-PLA2, and LAL. In one embodiment, the CHO cell lipase is PLBL2. In another embodiment, the CHO cell lipase is LPL. In yet another embodiment, the CHO cell lipase is LPLA2. In one embodiment, the CHO cell lipase is LP-PLA2. In another embodiment, the CHO cell lipase is LAL. In still another embodiment, the CHO cell lipase includes two, three, four, five, six, seven, eight, nine, ten, or more different CHO cell lipases. In yet still another embodiment, the CHO cell lipase includes two, three, four, or five different CHO cell lipases selected from the group consisting of PLBL2, LPL, LPLA2, LP-PLA2, and LAL. In one embodiment, the CHO cell lipase includes PLBL2 and LPL. In another embodiment, the CHO cell lipase includes PLBL2 and LPLA2. In yet another embodiment, the CHO cell lipase includes PLBL2 and LP-PLA2. In still another embodiment, the CHO cell lipase includes PLBL2 and LAL. In one embodiment, the CHO cell lipase includes LPL and LPLA2. In another embodiment, the CHO cell lipase includes LPL and LP-PLA2. In yet another embodiment, the CHO cell lipase includes LPL and LAL. In still another embodiment, the CHO cell lipase includes LPLA2 and LP-PLA2. In one embodiment, the CHO cell lipase includes LPLA2 and LAL. In another embodiment, the CHO cell lipase includes LP-PLA2 and LAL. In yet another embodiment, the CHO cell lipase includes PLBL2, LPL, and LPLA2. In still another embodiment, the CHO cell lipase includes PLBL2, LPL, and LP-PLA2. In one embodiment, the CHO cell lipase includes PLBL2, LPL, and LAL. In another embodiment, the CHO cell lipase includes PLBL2, LPLA2, and LP-PLA2. In yet another embodiment, the CHO cell lipase includes PLBL2, LPLA2, and LAL. In still another embodiment, the CHO cell lipase includes PLBL2, LP-PLA2, and LAL. In one embodiment, the CHO cell lipase includes LPL, LPLA2, and LP-PLA2. In another embodiment, the CHO cell lipase includes LPL, LPLA2, and LAL. In yet another embodiment, the CHO cell lipase includes LPL, LP-PLA2, and LAL. In still another embodiment, the CHO cell lipase includes LPLA2, LP-PLA2, and LAL. In one embodiment, the CHO cell lipase includes PLBL2, LPL, LPLA2, and LP-PLA2. In another embodiment, the CHO cell lipase includes PLBL2, LPL, LPLA2, and LAL. In yet another embodiment, the CHO cell lipase includes PLBL2, LPL, LP-PLA2, and LAL. In still another embodiment, the CHO cell lipase includes PLBL2, LPLA2, LP-PLA2, and LAL. In yet still another embodiment, the CHO cell lipase includes PLBL2, LPL, LPLA2, LP-PLA2, and LAL.

In some embodiments of various methods provided herein, the chromatographic resin is an IEX resin. In other embodiments, the chromatographic resin is a HIC resin. In one embodiment, the IEX resin is a CEX resin. In another embodiment, the CEX resin is a mixed mode CEX resin. In yet another embodiment, the IEX resin is an AEX resin. In still another embodiment, the AEX resin is a mixed mode AEX resin.

In certain embodiments of various methods using a CEX resin, the pH of the operating condition is below about 6.0. In some embodiments, the pH of the operating condition is below about 5.5. In other embodiments, the pH of the operating condition is below about 5.0. In yet other embodiments, the pH of the operating condition is from about 4.5 to about 5.5. In still other embodiments, the pH of the operating condition is from about 4.5 to about 5.0. In certain embodiments, the pH of the operating condition is from about 5.0 to about 5.5. In some embodiments, the pH of the operating condition is from about 4.9 to about 5.3.

In certain embodiments of various methods using a mixed mode CEX resin, the pH of the operating condition is below about 6.0. In some embodiments, the pH of the operating condition is below about 5.5. In other embodiments, the pH of the operating condition is below about 5.0. In yet other embodiments, the pH of the operating condition is from about 4.5 to about 5.5. In still other embodiments, the pH of the operating condition is from about 4.5 to about 5.0. In certain embodiments, the pH of the operating condition is from about 5.0 to about 5.5. In some embodiments, the pH of the operating condition is from about 4.9 to about 5.3.

In certain embodiments of various methods using an AEX resin, the pH of the operating condition is above about 6.5. In some embodiments, the pH of the operating condition is above about 6.9. In other embodiments, the pH of the operating condition is above about 7.2. In yet other embodiments, the pH of the operating condition is from about 6.9 to about 7.9. In still other embodiments, the pH of the operating condition is from about 7.2 to about 7.5. In certain embodiments, the pH of the operating condition is from about 7.5 to about 7.8.

In certain embodiments of various methods using a mixed mode AEX resin, the pH of the operating condition is above about 6.5. In some embodiments, the pH of the operating condition is above about 6.9. In other embodiments, the pH of the operating condition is above about 7.2. In yet other embodiments, the pH of the operating condition is from about 6.9 to about 7.9. In still other embodiments, the pH of the operating condition is from about 7.2 to about 7.5. In certain embodiments, the pH of the operating condition is from about 7.5 to about 7.8.

In certain embodiments of various methods provided herein, the operating condition further comprises modulating ionic strength and/or conductivity by adding a salt. In one embodiment, the operating condition further comprises modulating ionic strength by adding a salt. In another embodiment, the operating condition further comprises modulating conductivity by adding a salt. In yet another embodiment, the operating condition further comprises modulating ionic strength and conductivity by adding a salt. In some embodiments, the effect of adding a salt is to achieve the desired log α. In other embodiments, the effect of adding a salt is to achieve the desired log K_{P} for the lipase. In yet other embodiments, the effect of adding a salt is to achieve the desired log α and the desired log K_{P} for the lipase. Thus, in one embodiment, the operating condition further comprises achieving the desired log α by adding a salt. In another embodiment, the operating condition further comprises achieving the desired log K_{P} for the lipase by adding a salt. In yet another embodiment, the operating condition further comprises achieving the desired log α and the desired log K_{P} for the lipase by adding a salt.

In some embodiments, the salt in the operating solution is selected from the group consisting of sodium chloride, sodium acetate, sodium phosphate, ammonium sulfate, sodium sulfate, and Tris-HCl. In one embodiment, the salt is sodium chloride. In another embodiment, the salt is sodium acetate. In yet another embodiment, the salt is sodium phosphate. In still another embodiment, the salt is ammonium sulfate. In one embodiment, the salt is sodium sulfate. In another embodiment, the salt is Tris-HCl.

In a specific embodiment, the concentration of sodium chloride in the operating solution is from about 100 mM to about 225 mM, the chromatographic resin is CEX, and the pH of the operating condition is from about 5.0 to about 6.0.

In another specific embodiment, the concentration of sodium chloride in the operating solution is from about 150 mM to about 180 mM, the chromatographic resin is CEX, and the pH of the operating condition is from about 5.0 to about 6.0.

In yet another specific embodiment, the concentration of sodium acetate in the operating solution is from about 100 mM to about 200 mM, the chromatographic resin is AEX, and the pH of the operating condition is from about 6.9 to about 7.8.

In still another specific embodiment, the concentration of sodium sulfate in the operating solution is from about 500 mM to about 620 mM, the chromatographic resin is HIC, and the pH of the operating condition is about 7.

In yet still another specific embodiment, the concentration of sodium sulfate in the operating solution is from about 510 mM to about 560 mM, the chromatographic resin is HIC, and the pH of the operating condition is about 7.

In yet another aspect, provided herein is a method of separating PLBL2 from a anti-CTLA4 antibody, or antigen binding fragment thereof, through a mixed mode AEX chromatographic process, comprising:
(a) passing a load fluid comprising PLBL2 and the anti-CTLA4 antibody, or antigen binding fragment thereof, through a mixed mode AEX resin; and
(b) collecting the anti-CTLA4 antibody, or antigen binding fragment thereof, in a flowthrough;
   wherein the pH of the load fluid is from about pH 7.2 to about pH 7.6, and wherein the load fluid does not comprise a salt.

In certain embodiments of such methods, the anti-CTLA4 antibody, or antigen binding fragment thereof, is a therapeutic protein.

In some embodiments of such methods, the anti-CTLA4 antibody, or antigen binding fragment thereof, is a monoclonal antibody.

In still another aspect, provided herein is a method of separating PLBL2 from an anti-CTLA4 antibody, or antigen binding fragment thereof, through a CEX chromatographic process, comprising:
(a) passing a load fluid comprising PLBL2 and the anti-CTLA4 antibody, or antigen binding fragment thereof, through a CEX resin; and
(b) eluting the anti-CTLA4 antibody, or antigen binding fragment thereof, from the CEX resin with an elution solution;
   wherein the pH of the elution solution is from about pH 4.9 to about pH 5.3, and wherein the elution solution further comprises from about 120 mM to about 175 mM sodium chloride.

In one embodiment, the method of separating PLBL2 from an anti-CTLA4 antibody, or antigen binding fragment thereof, through a CEX chromatographic process comprises:
(a) passing a load fluid comprising PLBL2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is about pH 5.1, and wherein the elution solution further comprises about 150 mM sodium chloride.

In yet another embodiment, the method of separating PLBL2 from an anti-CTLA4 antibody, or antigen binding fragment thereof, through a CEX chromatographic process comprises:
(a) passing a load fluid comprising PLBL2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is about pH 5.1, and wherein the elution solution further comprises about 165 mM sodium chloride.

In still another aspect, provided herein is a method of separating LPLA2 from an anti-CTLA4 antibody, or antigen binding fragment thereof, through a CEX chromatographic process, comprising:
(a) passing a load fluid comprising LPLA2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is from about pH 5.0 to about pH 5.4, and wherein the elution solution further comprises from about 150 mM to about 275 mM sodium chloride.

In one embodiment, the method of separating LPLA2 from an anti-CTLA4 antibody, or antigen binding fragment thereof, through a CEX chromatographic process comprises:
(a) passing a load fluid comprising LPLA2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is about pH 5.1, and wherein the elution solution further comprises about 150 mM sodium chloride.

In another embodiment, the method of separating LPLA2 from an anti-CTLA4 antibody, or antigen binding fragment thereof, through a CEX chromatographic process comprises:
(a) passing a load fluid comprising LPLA2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is about pH 5.1, and wherein the elution solution further comprises about 200 mM sodium chloride.

In yet another embodiment, the method of separating LPLA2 from an anti-CTLA4 antibody, or antigen binding fragment thereof, through a CEX chromatographic process comprises:
(a) passing a load fluid comprising LPLA2 and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is about pH 5.1, and wherein the elution solution further comprises about 250 mM sodium chloride.

The methods of separation provided herein can be used in combination with one or more separation steps described herein or commonly used in the art. In one embodiment, one or more separation steps precede the method described herein. In another embodiment, one or more separation steps follow the method described herein. In yet another embodiment, one or more separation steps are performed between two methods described herein. In still other embodiments, one or more separation steps are performed before, after, and/or between the methods described herein. There is no limitation of how many separation steps or methods can be combined or the order of the separation steps or methods to be combined.

In more embodiments of the various methods provided herein, the load fluid is an eluate from a prior chromatographic process. In one embodiment, the prior chromatographic process comprises an affinity chromatography. In another embodiment, the prior chromatographic process comprises an affinity chromatography followed by a non-affinity chromatography. In yet another embodiment, the affinity chromatography is a protein A chromatography. In still another embodiment, the non-affinity chromatography is an AEX chromatography. In yet still another embodiment, the prior chromatographic process comprises a protein A chromatography followed by an AEX chromatography.

In yet still another aspect, provided herein is a method of separating a host cell lipase from an anti-CTLA4 antibody, or antigen binding fragment thereof, through a CEX chromatographic process, comprising:
(a) passing a load fluid comprising the host cell lipase and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is from about pH 4.9 to about pH 5.4, and wherein the conductivity of the elution solution is from about 15 mS/cm to about 21 mS/cm.

In certain embodiments of such a method, the anti-CTLA4 antibody, or antigen binding fragment thereof, is a therapeutic protein.

In other embodiments of such a method, the anti-CTLA4 antibody, or antigen binding fragment thereof, is a monoclonal antibody.

In still another aspect, provided herein is a method of separating a host cell lipase from an anti-CTLA4 antibody, or antigen binding fragment thereof, through a CEX chromatographic process, comprising:
(a) passing a load fluid comprising the host cell lipase and the anti-CTLA4 antibody, or antigen binding fragment thereof, through a CEX resin; and
(b) eluting the anti-CTLA4 antibody, or antigen binding fragment thereof, from the CEX resin with an elution solution;

wherein the pH of the elution solution is from about pH 4.9 to about pH 5.4, and wherein the elution solution further comprises from about 135 mM to about 195 mM sodium chloride.

### 7. Methods of Improving PS-80 Stability in a Production Protein Formulation

This disclosure further provides methods of improving PS-80 stability in a production protein formulation (e.g., drug substance formulation or drug product formulation) by separating a HCP (e.g., lipase) from the production protein (e.g., monoclonal antibody) using a chromatographic process.

In yet still another aspect, provided herein is a method of improving PS-80 stability in an anti-CTLA4 antibody, or antigen binding fragment thereof, formulation, comprising:
(a) passing a load fluid comprising a host cell lipase and the anti-CTLA4 antibody, or antigen binding fragment thereof, through a chromatographic resin under a loading operating condition;
(b) collecting the anti-CTLA4 antibody, or antigen binding fragment thereof, in a flowthrough; and
(c) formulating the anti-CTLA4 antibody, or antigen binding fragment thereof, so that the anti-CTLA4 antibody, or antigen binding fragment thereof, formulation comprises PS-80;
   wherein separation factor (α) is the ratio of the partition coefficient (K_{P}) for the lipase to the K_{P} for the anti-CTLA4 antibody, or antigen binding fragment thereof, and wherein log α is larger than 0.5 under the loading operating condition.

In certain embodiments, log α is larger than 1.0 under the loading operating condition.

In some embodiments, the log K_{P} for the lipase is larger than 1.0 under the loading operating condition. In other embodiments, the log K_{P} for the lipase is larger than 1.5 under the loading operating condition.

In certain embodiments, log α is larger than 0.5 and the log K_{P} for the lipase is larger than 1.0 under the loading operating condition. In some embodiments, log α is larger than 0.5 and the log K_{P} for the lipase is larger than 1.5 under the loading operating condition. In other embodiments, log α is larger than 1.0 and the log K_{P} for the lipase is larger than 1.0 under the loading operating condition. In yet other embodiments, log α is larger than 1.0 and the log K_{P} for the lipase is larger than 1.5 under the loading operating condition.

In another aspect, provided herein is a method of improving PS-80 stability in an anti-CTLA4 antibody, or antigen binding fragment thereof, formulation, comprising:
(a) passing a load fluid comprising a host cell lipase and the anti-CTLA4 antibody, or antigen binding fragment thereof, through a chromatographic resin;
(b) eluting the anti-CTLA4 antibody, or antigen binding fragment thereof, from the chromatographic resin with an elution solution under an elution operating condition; and
(c) formulating the anti-CTLA4 antibody, or antigen binding fragment thereof, so that the anti-CTLA4 antibody, or antigen binding fragment thereof, is in a solution containing PS-80;
   wherein α is the ratio of K_{P} for the lipase to the K_{P} for the anti-CTLA4 antibody, or antigen binding fragment thereof, and wherein log α is larger than 0.5 under the elution operating condition.

In certain embodiments, log α is larger than 1.0 under the elution operating condition.

In some embodiments, the log K_{P} for the lipase is larger than 1.0 under the elution operating condition. In other embodiments, the log K_{P} for the lipase is larger than 1.5 under the elution operating condition.

In certain embodiments, log α is larger than 0.5 and the log K_{P} for the lipase is larger than 1.0 under the elution operating condition. In some embodiments, log α is larger than 0.5 and the log K_{P} for the lipase is larger than 1.5 under the elution operating condition. In other embodiments, log α is larger than 1.0 and the log K_{P} for the lipase is larger than 1.0 under the elution operating condition. In yet other embodiments, log α is larger than 1.0 and the log K_{P} for the lipase is larger than 1.5 under the elution operating condition.

In some embodiments of various methods provided herein, the lipase is a Chinese Hamster Ovary (CHO) cell lipase.

In certain embodiments, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

In certain embodiments, the CHO cell lipase is selected from the group consisting of PLBL2, LPL, LPLA2, LP-PLA2, and LAL. In one embodiment, the CHO cell lipase is PLBL2. In another embodiment, the CHO cell lipase is LPL. In yet another embodiment, the CHO cell lipase is LPLA2. In one embodiment, the CHO cell lipase is LP-PLA2. In another embodiment, the CHO cell lipase is LAL. In still another embodiment, the CHO cell lipase includes two, three, four, five, six, seven, eight, nine, ten, or more different CHO cell lipases. In yet still another embodiment, the CHO cell lipase includes two, three, four, or five different CHO cell lipases selected from the group consisting of PLBL2, LPL, LPLA2, LP-PLA2, and LAL. In one embodiment, the CHO cell lipase includes PLBL2 and LPL. In another embodiment, the CHO cell lipase includes PLBL2 and LPLA2. In yet another embodiment, the CHO cell lipase includes PLBL2 and LP-PLA2. In still another embodiment, the CHO cell lipase includes PLBL2 and LAL. In one embodiment, the CHO cell lipase includes LPL and LPLA2. In another embodiment, the CHO cell lipase includes LPL and LP-PLA2. In yet another embodiment, the CHO cell lipase includes LPL and LAL. In still another embodiment, the CHO cell lipase includes LPLA2 and LP-PLA2. In one embodiment, the CHO cell lipase includes LPLA2 and LAL. In another embodiment, the CHO cell lipase includes LP-PLA2 and LAL. In yet another embodiment, the CHO cell lipase includes PLBL2, LPL, and LPLA2. In still another embodiment, the CHO cell lipase includes PLBL2, LPL, and LP-PLA2. In one embodiment, the CHO cell lipase includes PLBL2, LPL, and LAL. In another embodiment, the CHO cell lipase includes PLBL2, LPLA2, and LP-PLA2. In yet another embodiment, the CHO cell lipase includes PLBL2, LPLA2, and LAL. In still another embodiment, the CHO cell lipase includes PLBL2, LP-PLA2, and LAL. In one embodiment, the CHO cell lipase includes LPL, LPLA2, and LP-PLA2. In another embodiment, the CHO cell lipase includes LPL, LPLA2, and LAL. In yet another embodiment, the CHO cell lipase includes LPL, LP-PLA2, and LAL. In still another embodiment, the CHO cell lipase includes LPLA2, LP-PLA2, and LAL. In one embodiment, the CHO cell lipase includes PLBL2, LPL, LPLA2, and LP-PLA2. In another embodiment, the CHO cell lipase includes PLBL2, LPL, LPLA2, and LAL. In yet another embodiment, the CHO cell lipase includes PLBL2, LPL, LP-PLA2, and LAL. In still another embodiment, the CHO cell lipase includes PLBL2, LPLA2, LP-PLA2, and LAL. In yet still another embodiment, the CHO cell lipase includes PLBL2, LPL, LPLA2, LP-PLA2, and LAL.

In some embodiments of various methods provided herein, the chromatographic resin is an ion exchange (IEX) resin. In other embodiments, the chromatographic resin is a hydrophobic interaction (HIC) resin. In one embodiment, the IEX resin is a cation exchange (CEX) resin. In another embodiment, the CEX resin is a mixed mode CEX resin. In yet another embodiment, the IEX resin is an anion exchange (AEX) resin. In still another embodiment, the AEX resin is a mixed mode AEX resin.

In certain embodiments of various methods using a CEX resin or a mixed mode CEX resin, the pH of the operating condition is below about 6.0. In some embodiments of various methods using a CEX resin or a mixed mode CEX resin, the pH of the operating condition is below about 5.5. In other embodiments of various methods using a CEX resin or a mixed mode CEX resin, the pH of the operating condition is below about 5.0. In yet other embodiments of various methods using a CEX resin or a mixed mode CEX resin, the pH of the operating condition is from about 4.5 to about 5.5. In still other embodiments of various methods using a CEX resin or a mixed mode CEX resin, the pH of the operating condition is from about 4.5 to about 5.0. In certain embodiments of various methods using a CEX resin or a mixed mode CEX resin, the pH of the operating condition is from about 5.0 to about 5.5. In some embodiments of various methods using a CEX resin or a mixed mode CEX resin, the pH of the operating condition is from about 4.9 to about 5.3.

In certain embodiments of various methods using an AEX resin or a mixed mode AEX resin, the pH of the operating condition is above about 6.5. In some embodiments of various methods using an AEX resin or a mixed mode AEX resin, the pH of the operating condition is above about 6.9. In other embodiments of various methods using an AEX resin or a mixed mode AEX resin, the pH of the operating condition is above about 7.2. In yet other embodiments of various methods using an AEX resin or a mixed mode AEX resin, the pH of the operating condition is from about 6.9 to about 7.9. In still other embodiments of various methods using an AEX resin or a mixed mode AEX resin, the pH of the operating condition is from about 7.2 to about 7.5. In certain embodiments of various methods using an AEX resin or a mixed mode AEX resin, the pH of the operating condition is from about 7.5 to about 7.8.

In certain embodiments of various methods provided herein, the operating condition further comprises modulating the ionic strength and/or conductivity of the operating solution by adding a salt. In one embodiment, the operating condition further comprises modulating the ionic strength of the operating solution by adding a salt. In another embodiment, the operating condition further comprises modulating the conductivity of the operating solution by adding a salt. In yet another embodiment, the operating condition further comprises modulating the ionic strength and conductivity of the operating solution by adding a salt. In some embodiments, the effect of adding a salt is to achieve the desired log α. In other embodiments, the effect of adding a salt is to achieve the desired log K_{P} for the lipase. In yet other embodiments, the effect of adding a salt is to achieve the desired log α and the desired log K_{P} for the lipase.

In some embodiments, the salt in the operating solution is selected from the group consisting of sodium chloride, sodium acetate, sodium phosphate, ammonium sulfate, sodium sulfate, and Tris-HCl. In one embodiment, the salt is sodium chloride. In another embodiment, the salt is sodium acetate. In yet another embodiment, the salt is sodium phosphate. In still another embodiment, the salt is ammonium sulfate. In one embodiment, the salt is sodium sulfate. In another embodiment, the salt is Tris-HCl.

In a specific embodiment, the concentration of sodium chloride in the operating solution is from about 100 mM to about 225 mM, the chromatographic resin is CEX, and the pH of the operating condition is from about 5.0 to about 6.0.

In another specific embodiment, the concentration of sodium chloride in the operating solution is from about 150 mM to about 180 mM, the chromatographic resin is CEX, and the pH of the operating condition is from about 5.0 to about 6.0.

In yet another specific embodiment, the concentration of sodium acetate in the operating solution is from about 100 mM to about 200 mM, the chromatographic resin is AEX; the pH of the operating condition is from about 6.9 to about 7.8.

In still another specific embodiment, the concentration of sodium sulfate in the operating solution is from about 500 mM to about 620 mM, the chromatographic resin is HIC, and the pH of the operating condition is about 7.

In yet still another specific embodiment, the concentration of sodium sulfate in the operating solution is from about 510 mM to about 560 mM, the chromatographic resin is HIC, and the pH of the operating condition is about 7.

In yet another aspect, provided herein is a method of improving PS-80 stability in an anti-CTLA4 antibody, or antigen binding fragment thereof, formulation, comprising:
(a) passing a load fluid comprising the anti-CTLA4 antibody, or antigen binding fragment thereof, through a mixed mode AEX resin;
(b) collecting the anti-CTLA4 antibody, or antigen binding fragment thereof, in a flowthrough; and
(c) formulating the anti-CTLA4 antibody, or antigen binding fragment thereof, so that the anti-CTLA4 antibody, or antigen binding fragment thereof, formulation is a solution comprising PS-80;

wherein the pH of the load fluid is from about pH 7.2 to about pH 7.6, and wherein the load fluid does not comprise a salt.

In another aspect, provided herein is a process for making an anti-CTLA4 antibody or antigen binding fragment thereof, formulation, comprising:
(a) passing a load fluid comprising the anti-CTLA4 antibody, or antigen binding fragment thereof, through a mixed mode AEX resin;
(b) collecting the anti-CTLA4 antibody, or antigen binding fragment thereof, in a flowthrough; and
(c) formulating the anti-CTLA4 antibody, or antigen binding fragment thereof obtained from step (b), in a solution comprising PS-80, wherein and the average PS80 degradation is less than or equal to 10% after six months.

In yet another embodiment, the PS80 degradation is measured by the amount of intact PS-80 molecules and/or the amount of degraded products using various methods, including but not limited to mass spectrometry (MS), liquid chromatography-mass spectrometry (LCMS), or solid phase extraction (SPE) on a HPLC system with a charged aerosol detector (CAD).

In still another aspect, provided herein is a method of improving PS-80 stability in an anti-CTLA4 antibody, or antigen binding fragment thereof, formulation, comprising:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a solution comprising PS-80;

wherein the pH of the elution solution is from about pH 4.9 to about pH 5.3, and wherein the elution solution further comprises from about 120 mM to about 175 mM sodium chloride.

In one embodiment, the method of improving PS-80 stability in an anti-CTLA4 antibody, or antigen binding fragment thereof, formulation comprises:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a solution comprising PS-80;

wherein the pH of the elution solution is about pH 5.1, and wherein the elution solution further comprises about 150 mM sodium chloride.

In yet another embodiment, the method of improving PS-80 stability in an anti-CTLA4 antibody, or antigen binding fragment thereof, formulation comprises:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a solution comprising PS-80;

wherein the pH of the elution solution is about pH 5.1, and wherein the elution solution further comprises about 165 mM sodium chloride.

In still another aspect, provided herein is a method of improving PS-80 stability in an anti-CTLA4 antibody, or antigen binding fragment thereof, formulation, comprising:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a solution comprising PS-80;

wherein the pH of the elution solution is from about pH 5.0 to about pH 5.4, and wherein the elution solution further comprises from about 150 mM to about 275 mM sodium chloride.

In one embodiment, the method of improving PS-80 stability in an anti-CTLA4 antibody, or antigen binding fragment thereof, formulation comprises:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a solution comprising PS-80;

wherein the pH of the elution solution is about pH 5.1, and wherein the elution solution further comprises about 200 mM sodium chloride.

In yet another embodiment, the method of improving PS-80 stability in an anti-CTLA4 antibody, or antigen binding fragment thereof, formulation comprises:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a solution comprising PS-80;

wherein the pH of the elution solution is about pH 5.1, and wherein the elution solution further comprises about 250 mM sodium chloride.

In more embodiments of the various methods provided herein, the load fluid is an eluate from a prior chromatographic process. In one embodiment, the prior chromatographic process comprises an affinity chromatography. In another embodiment, the prior chromatographic process comprises an affinity chromatography followed by a non-affinity chromatography. In yet another embodiment, the affinity chromatography is a protein A chromatography. In still another embodiment, the non-affinity chromatography is an AEX chromatography. In yet still another embodiment, the prior chromatographic process comprises a protein A chromatography followed by an AEX chromatography.

In another aspect, provided herein is a method of improving PS-80 stability in an anti-CTLA4 antibody, or antigen binding fragment thereof, formulation, comprising:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a solution comprising PS-80;

wherein the pH of the elution solution is from about pH 4.9 to about pH 5.4, and wherein the conductivity of the elution solution is from about 15 mS/cm to about 21 mS/cm.

In another aspect, provided herein is a method of improving PS-80 stability in an anti-CTLA4 antibody, or antigen binding fragment thereof, formulation, comprising:
(a) passing a load fluid comprising the anti-CTLA4 antibody, or antigen binding fragment thereof, through a CEX resin;
(b) eluting the anti-CTLA4 antibody, or antigen binding fragment thereof, from the CEX resin with an elution solution; and
(c) formulating the anti-CTLA4 antibody, or antigen binding fragment thereof, so that the anti-CTLA4 antibody, or antigen binding fragment thereof, formulation is a solution comprising PS-80;

wherein the pH of the elution solution is from about pH 4.9 to about pH 5.4, and wherein the elution solution further comprises from about 135 mM to about 195 mM sodium chloride.

### 8. Pharmaceutical Compositions

This disclosure also provides pharmaceutical compositions (e.g., drug substance or drug product) comprising a therapeutic protein (e.g., monoclonal antibody) and little amount of an HCP (e.g., lipase).

In certain embodiments, the pharmaceutical composition comprises a therapeutic protein and less than 1 ppm of a host cell lipase. In other embodiments, the pharmaceutical composition comprises a therapeutic protein and less than 0.1, 0.2, 0.3, 0.4, 0.5, 0.6, 0.7, 0.8, or 0.9 ppm of a host cell lipase. In one embodiment, the pharmaceutical composition comprises a therapeutic protein and less than 0.1 ppm of a host cell lipase. In another embodiment, the pharmaceutical composition comprises a therapeutic protein and less than 0.2 ppm of a host cell lipase. In yet another embodiment, the pharmaceutical composition comprises a therapeutic protein and less than 0.3 ppm of a host cell lipase. In still another embodiment, the pharmaceutical composition comprises a therapeutic protein and less than 0.4 ppm of a host cell lipase. In yet still another embodiment, the pharmaceutical composition comprises a therapeutic protein and less than 0.5 ppm of a host cell lipase. In one embodiment, the pharmaceutical composition comprises a therapeutic protein and less than 0.6 ppm of a host cell lipase. In another embodiment, the pharmaceutical composition comprises a therapeutic protein and less than 0.7 ppm of a host cell lipase. In yet another embodiment, the pharmaceutical composition comprises a therapeutic protein and less than 0.8 ppm of a host cell lipase. In still another embodiment, the pharmaceutical composition comprises a therapeutic protein and less than 0.9 ppm of a host cell lipase.

In various embodiments of the pharmaceutical compositions described herein, the level of the host cell lipase is measured by liquid chromatography-mass spectrometry (LC-MS).

In certain embodiments, the pharmaceutical composition is an eluate from a CEX chromatography using an elution solution selected from the group consisting of:
(a) an elution solution with a pH from about 4.9 to about 5.3, comprising from about 120 mM to about 175 mM sodium chloride;
(b) an elution solution with a pH of about 5.1, comprising about 150 mM sodium chloride;
(c) an elution solution with a pH of about 5.1, comprising about 165 mM sodium chloride;
(d) an elution solution with a pH from about 4.9 to about 5.4 and a conductivity from about 15 mS/cm to about 21 mS/cm;
(e) an elution solution with a pH from about 4.9 to about 5.4, comprising from about 135 mM to about 195 mM sodium chloride;
(f) an elution solution with a pH from about pH 5.0 to about pH 5.4, comprising from about 150 mM to about 275 mM sodium chloride;
(g) an elution solution with a pH of about 5.1, comprising about 200 mM sodium chloride; and
(h) an elution solution with a pH of about 5.1, comprising about 250 mM sodium chloride.

In one embodiment, the pharmaceutical composition is an eluate from a CEX chromatography using an elution solution with a pH from about 4.9 to about 5.3, comprising from about 120 mM to about 175 mM sodium chloride.

In another embodiment, the pharmaceutical composition is an eluate from a CEX chromatography using an elution solution with a pH of about 5.1, comprising about 150 mM sodium chloride.

In yet another embodiment, the pharmaceutical composition is an eluate from a CEX chromatography using an elution solution with a pH of about 5.1, comprising about 165 mM sodium chloride.

In still another embodiment, the pharmaceutical composition is an eluate from a CEX chromatography using an elution solution with a pH from about 4.9 to about 5.4 and a conductivity from about 15 mS/cm to about 21 mS/cm.

In one embodiment, the pharmaceutical composition is an eluate from a CEX chromatography using an elution solution with a pH from about 4.9 to about 5.4, comprising from about 135 mM to about 195 mM sodium chloride.

In another embodiment, the pharmaceutical composition is an eluate from a CEX chromatography using an elution solution with a pH from about pH 5.0 to about pH 5.4, comprising from about 150 mM to about 275 mM sodium chloride.

In still another embodiment, the pharmaceutical composition is an eluate from a CEX chromatography using an elution solution with a pH of about 5.1, comprising about 200 mM sodium chloride.

In yet still another embodiment, the pharmaceutical composition is an eluate from a CEX chromatography using an elution solution with a pH of about 5.1, comprising about 250 mM sodium chloride.

In some embodiments of the pharmaceutical compositions, the CEX chromatography is preceded by an AEX chromatography operated in a flowthrough mode.

In certain embodiments of the pharmaceutical compositions, the lipase is selected from the group consisting of PLBL2, LPL, LPLA2, LP-PLA2, and LAL. In one embodiment, the lipase is PLBL2. In another embodiment, the lipase is LPL. In yet another embodiment, the lipase is LPLA2. In one embodiment, the lipase is LP-PLA2. In another embodiment, the lipase is LAL. In still another embodiment, the lipase includes two, three, four, five, six, seven, eight, nine, ten, or more different lipases. In yet still another embodiment, the lipase includes two, three, four, or five different lipases selected from the group consisting of PLBL2, LPL, LPLA2, LP-PLA2, and LAL. In one embodiment, the lipase includes PLBL2 and LPL. In another embodiment, the lipase includes PLBL2 and LPLA2. In yet another embodiment, the lipase includes PLBL2 and LP-PLA2. In still another embodiment, the lipase includes PLBL2 and LAL. In one embodiment, the lipase includes LPL and LPLA2. In another embodiment, the lipase includes LPL and LP-PLA2. In yet another embodiment, the lipase includes LPL and LAL. In still another embodiment, the lipase includes LPLA2 and LP-PLA2. In one embodiment, the lipase includes LPLA2 and LAL. In another embodiment, the lipase includes LP-PLA2 and LAL. In yet another embodiment, the lipase includes PLBL2, LPL, and LPLA2. In still another embodiment, the lipase includes PLBL2, LPL, and LP-PLA2. In one embodiment, the lipase includes PLBL2, LPL, and LAL. In another embodiment, the lipase includes PLBL2, LPLA2, and LP-PLA2. In yet another embodiment, the lipase includes PLBL2, LPLA2, and LAL. In still another embodiment, the lipase includes PLBL2, LP-PLA2, and LAL. In one embodiment, the lipase includes LPL, LPLA2, and LP-PLA2. In another embodiment, the lipase includes LPL, LPLA2, and LAL. In yet another embodiment, the lipase includes LPL, LP-PLA2, and LAL. In still another embodiment, the lipase includes LPLA2, LP-PLA2, and LAL. In one embodiment, the lipase includes PLBL2, LPL, LPLA2, and LP-PLA2. In another embodiment, the lipase includes PLBL2, LPL, LPLA2, and LAL. In yet another embodiment, the lipase includes PLBL2, LPL, LP-PLA2, and LAL. In still another embodiment, the lipase includes PLBL2, LPLA2, LP-PLA2, and LAL. In yet still another embodiment, the lipase includes PLBL2, LPL, LPLA2, LP-PLA2, and LAL.

In other embodiments of the pharmaceutical compositions, the therapeutic protein is a monoclonal antibody.

### VI. EXAMPLES

The examples in this section (section VI) are offered by way of illustration, and not by way of limitation.

### Example 1: Method for Determining K_{P} of Different Species

A partitioning coefficient, K_{P}, is determined by mixing a known liquid concentration of protein (or other molecule of interest) with a known volume of chromatography resin and calculating the ratio of the protein bound to the resin and the protein remaining in the liquid: K_{P}=q/c=[bound]/[free].

For all subsequent examples, the chromatography volume was 20 μL, and the liquid volume was 200 μL with a protein concentration of 0.5 mg/mL. These volumes provide a phase ratio of 10:1 for an effective resin loading of 5 mg/mL.

Screenings were conducted by vigorous mixing of resin and liquid in a 96-well filter plate (P/N MSBVN1250, Millipore Sigma, Burlington, MA) with separation of resin and liquid by vacuum filtration. The sequence of steps was as follows:
(a) 3× equilibration (buffer not containing feed), 10 min incubation each step;
(b) 1× feed mixing, 60 min incubation; and
(c) 2× strip conditions, 10 min incubation each step

The equilibration step allows for buffer exchange from the initial resin slurry buffer. The 60 min time for feed mixing allows for pseudo equilibration between the resin ligand and protein at a given set of conditions. The filtrate from the feed step was measured by UV absorbance at 280-320 nm to determine the final liquid concentration of the protein, c. The bound concentration of the protein, q, was determined by a mass balance around c and the known feed concentration, c₀ (0.5 mg/mL).

Partitioning is generally reported in terms of log K_{P}, which can be accurately quantified from approximately 0 to 2 using the UV method described here. General rules for log K_{P} screening are as follows:
log K_{P}≥1.5, strong binding to the resin;
log K_{P}<1, conditions where elution would be expected for a bind-and-elute modality;
0.5<log K_{P}<1, weak interaction conditions that will show some binding;
log K_{P}<0.5, very little or no binding.

The log K_{P} of different species is also used to predict separation of different species through the calculation of a separation factor, α, as follows: α=K_{P, protein 1}/K_{P, protein 2}; log α=log K_{P, protein 1}−log K_{P, protein 2}, where a log α further from 0 indicates better separation. In the following examples, α=K_{P, lipase}/K_{P, mAb}; log α=log K_{P, lipase}−log K_{P, mAb}. A log α larger than 0.5 indicates good separation between the lipase and a monoclonal antibody.

### Example 2: Comparison of PLBL2 and mAb K_{P} Values at Typical Processing Conditions

The method for determining K_{P} and α was used to assess the capability of separating a known lipase impurity, PLBL2, at operating conditions for two monoclonal antibodies, mAb1 and mAb2, through a variety of chromatographic processes. Table 1 summarizes the log K_{P} and log α values for mAb1 and PLBL2 at several process conditions for mAb1.

TABLE 1
log K_{P} and log α values for mAb1 and PLBL2 at processing conditions for mAb1

| | | mAb1 | PLBL2 | log |
|---|---|---|---|---|
| Process, Resin | Operating Condition | log K_{P} | log K_{P} | α |
| Protein A, MabSelect Sure | Equil/wash: 10 mM NaPhosphate, | 2 | 0 | −2 |
| (GE Healthcare, Chicago, IL) | pH 6.5 | | | |
| | High salt wash: 10 mM | 2 | 0 | −2 |
| | NaPhosphate, pH 6.5, 0.5M NaCl | | | |
| | Elute: 20 mM NaAcetate, pH 3.5 | 0 | 0 | 0 |
| | Strip: 100 mM acetic acid | 0 | 0 | 0 |
| AEX, POROS HQ 50 | Load: 100 mM NaAcetate, 100 mM | 0.3 | 1.6 | 1.3 |
| (Thermo Fisher Scientific, | Tris, pH 7.5 | | | |
| Waltham, MA) | Wash: 25 mM NaPhosphate, pH 7.2 | 0 | 1.4 | 1.4 |
| | Strip: 1M NaCl | 0 | 0 | 0 |
| CEX, POROS HS 50 | Load: 100 mM NaAcetate, 100 mM | 2 | 2 | 0 |
| (Thermo Fisher Scientific, | Tris, pH 5.1 | | | |
| Waltham, MA) | Elute (low salt limit): 20 mM | 1.5 | 1.8 | 0.3 |
| | NaAcetate, pH 5.1, 125 mM NaCl | | | |
| | Elute (center point): 20 mM | 0.5 | 1.6 | 1.1 |
| | NaAcetate, pH 5.1, 150 mM NaCl | | | |
| | Elute (high salt limit): 20 mM | 0 | 1.4 | 1.4 |
| | NaAcetate, pH 5.1, 175 mM NaCl | | | |
| | Strip: 1M NaCl | 0 | 0 | 0 |

Table 1 shows potential operating conditions for separating PLBL2 from mAB1 through a chromatographic process.

For the protein A process, PLBL2 has no affinity, so the majority of PLBL2 would be expected to flow through the protein A resin during loading or wash steps. The only PLBL2 present in pools would likely be from insufficient washes or associated with mAb1.

For the AEX process, which is operated in flowthrough mode for mAb1, PLBL2 shows stronger binding (higher log K_{P}) compared to mAb1 at loading and wash conditions. This results in a log α larger than 1.0 at these conditions, which indicates that PLBL2 would remain bound to the resin at these conditions, whereas mAb1 would flow through.

For the CEX process, PLBL2 shows less sensitivity to salt modulation and has a higher log K_{P} at the higher ends of the salt range, compared to mAb1. A log α larger than 1.0 at the center point and high salt limit indicates that PLBL2 would remain bound during mAb1 elution. The low salt limit provides a much less favorable log α for separation of PLBL2 from mAb1, which is expected to retain some binding at these conditions (log K_{P, mAb1} of 1.5).

Table 2 summarizes the log K_{P} and log α values for mAb2 and PLBL2 at several process conditions for mAb2.

TABLE 2
log K_{P} and log α values for mAb2 and PLBL2 at processing conditions for mAb2

| | | mAb2 | PLBL2 | log |
|---|---|---|---|---|
| Process, Resin | Operating Condition | log K_{P} | log K_{P} | α |
| Protein A, MabSelect Sure | Equil/wash: 10 mM NaPhosphate, | 2 | 0 | −2 |
| | pH 6.5 | | | |
| | High salt wash: 10 mM | 2 | 0 | −2 |
| | NaPhosphate, pH 6.5, 0.5M NaCl | | | |
| | Elute: 20 mM NaAcetate, pH 3.5 | 0.3 | 0 | −0.3 |
| | Strip: 100 mM acetic acid | 0 | 0 | 0 |
| CEX, POROS HS 50 | Load: 100 mM NaAcetate, 100 mM | 2 | 2 | 0 |
| | Tris, pH 5.1 | | | |
| | Elute (low salt limit): 20 mM | 0.2 | 1.8 | 1.6 |
| | NaAcetate, pH 5.1, 125 mM NaCl | | | |
| | Elute (center point): 20 mM | 0 | 1.6 | 1.6 |
| | NaAcetate, pH 5.1, 150 mM NaCl | | | |
| | Elute (high salt limit): 20 mM | 0 | 1.4 | 1.4 |
| | NaAcetate, pH 5.1, 175 mM NaCl | | | |
| | Strip: 1M NaCl | 0 | 0 | 0 |

Table 2 shows potential operating conditions for separating PLBL2 from mAb2 through a chromatographic process.

The trends of mAb2 for the protein A and CEX processes are very similar to that of mAb1, with mAb2 demonstrating slightly stronger binding during the protein A elution and weaker binding during the CEX elution. For the CEX process, mAb2 has lower binding at lower salt and therefore a more robust log α throughout the salt range.

### Example 3: Mapping of PLBL2 and LPLA2 K_{P} Values at a Range of Conditions for Different Resins

An extensive mapping was performed to determine the partitioning coefficient of PLBL2 and LPLA2 for a wide variety of resins with different buffers and conditions that might potentially be used in downstream processing (Table 3). Salt and pH conditions were tested combinatorially. The comprehensive mapping of PLBL2 and LPLA2 K_{P} can provide a basis for predicting separation of PLBL2 or LPLA2 with known mAb purification conditions or for conditions to be explored.

TABLE 3
Conditions screened for mapping PLBL2 or LPLA2 log K_{P}

| Modality | Buffer | Salt | Salt Concentration (mM) | pH |
|---|---|---|---|---|
| AEX | Tris- | sodium | 50, 100, 150 | 7.0, 7.2, 7.5 |
| | acetate | acetate | | |
| | Tris | sodium | 0, 50, 100, 150 | 6.9, 7.2, 7.5, 7.8 |
| | | chloride | | |
| | Phosphate | sodium | 0, 25, 50 | 6.9, 7.2 |
| | | chloride | | |
| CEX - salt screen | Acetate | sodium | 100, 125, 150, 175, 200, 225, | 5.0, 5.2, 5.4, 5.6 |
| | | chloride | 250, 275, 300 | |
| CEX - pH screen | Acetate or | sodium | 0, 25, 50 | 5.0, 5.5, 6.0, 6.4, |
| | phosphate | chloride | | 6.8, 7.2, 7.6 |
| HIC | Phosphate | sodium | 0, 100, 150, 200, 250, 300, | 7.0 |
| | | sulfate | 350, 400, 450, 500, 550, 600 | |
| Mixed mode AEX | Tris | sodium | 0, 25, 50, 100, 150, 200 | 7.2, 7.5, 7.8 |
| | | chloride | | |
| Mixed mode CEX | Acetate | sodium | 0, 100, 150, 200, 250, 300, | 5.0, 5.5, 6.0 |
| | | chloride | 400, 500 | |

3.1 AEX Chromatography

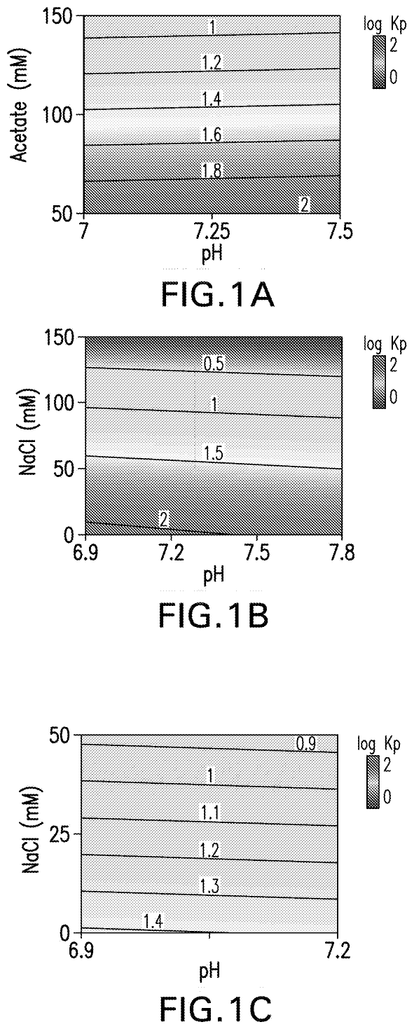

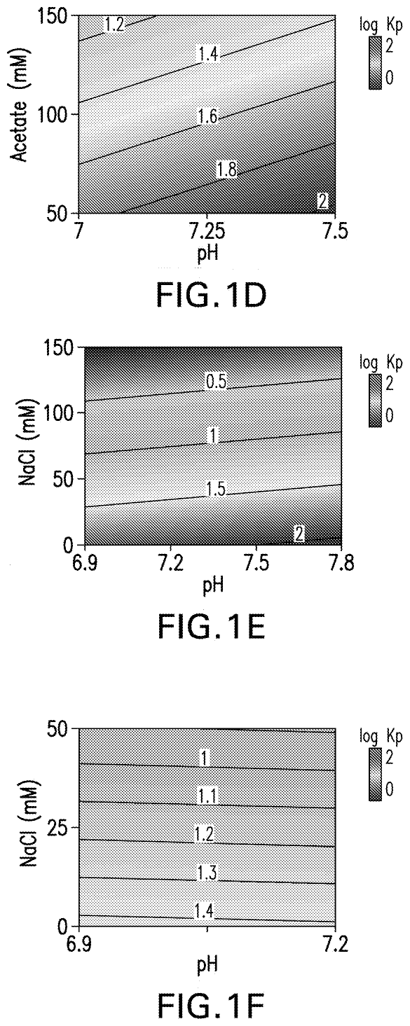

A set of conditions for AEX chromatography is listed in Table 3 and depicted in FIGS. 1A-1C for PLBL2 and in FIGS. 1D-1F for LPLA2 with POROS 50 HQ resin. The first buffer combination represents a mixture of buffers in ranges that might commonly be seen for AEX loading steps in flowthrough mode following protein A and low pH hold steps (FIGS. 1A and 1D). Under these conditions, acetate acted as the counter ion to compete for binding, and Tris base was added in appropriate amounts to control pH. PLBL2 showed strong interactions even at >100 mM acetate additions, with a log K_{P} of approximately 1 observed at about 140 mM acetate with log K_{P} presumably continuing to drop at higher acetate concentrations (FIG. 1A). Little difference was observed across the pH 7.0-7.5 range (FIG. 1A). LPLA2 showed similar trends with acetate but also showed a stronger dependence on pH with stronger binding seen at higher pH (FIG. 1D).

Two other AEX conditions represent buffers (Tris and Phosphate) that might be used for AEX equilibration and wash steps. The use of NaCl salt modulation provides possible conditions for AEX process following a prior salt elution (e.g., from CEX). In Tris buffer (FIGS. 1B and 1E), PLBL2 remains strongly bound (log K_{P}>1.5) up to about 50 mM NaCl addition, and log K_{P} drops below 1 above approximately 100 mM NaCl. LPLA2 behaves similar but is less strongly retained with log K_{P}>1.5 up to about 30 mM NaCl and log K_{P}<1 above about 75 mM NaCl. The phosphate buffer (FIGS. 1C and 1F) prevents PLBL2 and LPLA2 interactions more strongly than Tris; no conditions screened with either lipase provided log K_{P}>1.5, and log K_{P} dropped below 1 at about 40 mM NaCl in each case. pH had little effect for either buffer across the ranges tested.

3.2 CEX Chromatography

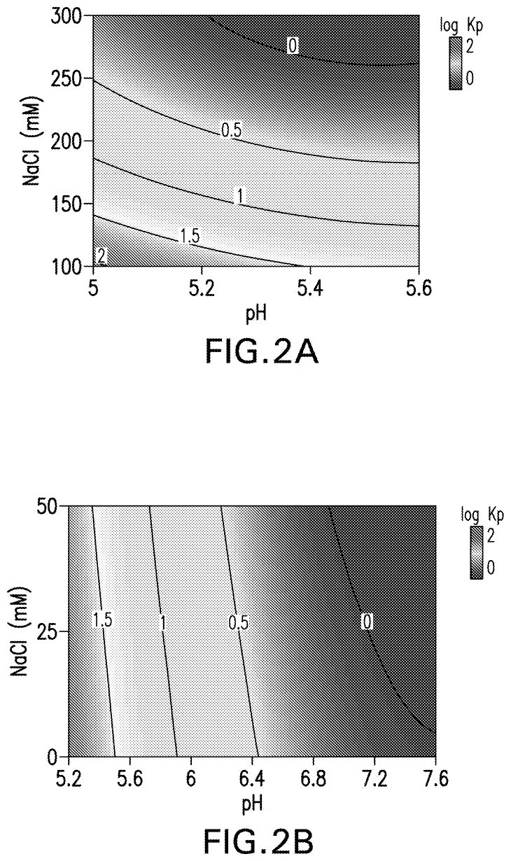

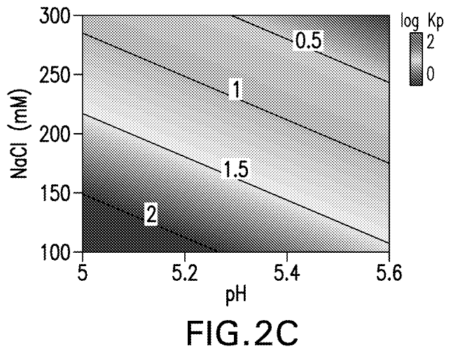

A set of conditions for CEX chromatography is listed in Table 3 and depicted in FIGS. 2A-2C for POROS 50 HS resin.

The first combination represents a pH and salt range that might typically be used for binding and elution of mAbs using NaCl modulation for elution (FIGS. 2A and 2C). In this case, NaCl had a strong effect with no binding seen above 250 mM NaCl and log K_{P} values of 1 around 150 mM NaCl for PLBL2 (FIG. 2A). pH also had a significant impact with increasing log K_{P} at lower pH, particularly closer to pH 5.0. LPLA2 demonstrated similar trends for pH and salt modulation but had significantly stronger retention with log K_{P} values of 1 between 200-250 mM NaCl (FIG. 2C).

The second combination represents conditions where pH is used to modulate binding, typically at much lower salt conditions (FIG. 2B). In this case, strong PLBL2 binding above log K_{P} of 1.5 was only seen below pH 5.5, and log K_{P} values drop below 1 above about pH 5.8. The salt conditions tested here had little impact on partitioning at these pH values. Acetate buffer was used up to pH 6.0, and phosphate buffer was used to buffer higher pH conditions.

3.3 HIC

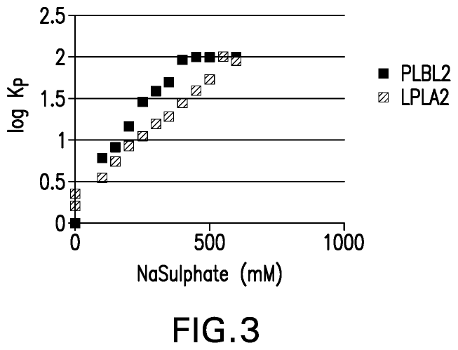

Testing for partitioning of PLBL2 and LPLA2 to a HIC resin, Tosoh Butyl-650M, was conducted by modulating sodium sulfate concentration at a buffering condition of 20 mM sodium phosphate (pH 7.0) (Table 3, FIG. 3). Both lipases showed typical HIC behavior with strong binding at high salt (log K_{P}>1.5 above 250 mM sodium sulfate for PLBL2 and 400 mM sodium sulfate for LPLA2) and decreased partitioning at lower salt (log K_{P}<1 below 150 mM sodium sulfate for PLBL2 and 200 mM sodium sulfate for LPLA2).

3.4 Multimodal Chromatography

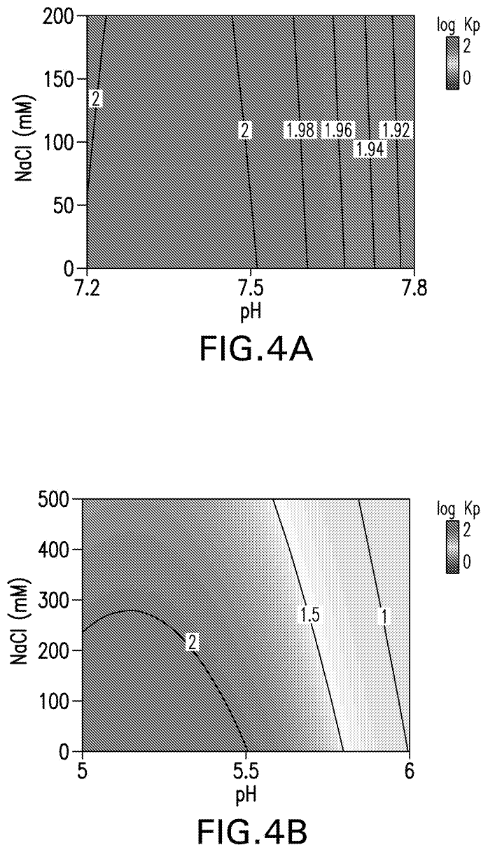

Partitioning of PLBL2 was also tested on two multimodal chromatography resins: a multimodal AEX resin, Capto adhere, and a multimodal CEX resin, Capto MMC (Table 3, FIGS. 4A and 4B). For Capto adhere, log K_{P} values were greater than 1.9 at all conditions tested, demonstrating strong binding over a wide range of operating conditions (FIG. 4A). For Capto MMC, binding was predominately modulated by pH changes across a much wider salt range (FIG. 4B). Strong binding range with log K_{P} above 1.5 was observed below about pH 5.8. Weaker binding range with log K_{P} less than 1 was only observed above pH of approximately 5.9 with high salt additions.

### Example 4: Optimization of Conditions to Separate PLBL2 from an IgG1 mAb, mAb3

The partitioning maps provided for PLBL2 in Example 3 were used to optimize separation of PLBL2 from an IgG1 mAb, mAb3. The performance of mAb3 was similar to that of mAb1 for the protein A process. Strong binding of mAb3 was observed under protein A loading conditions (data not shown).

4.1 AEX Chromatography

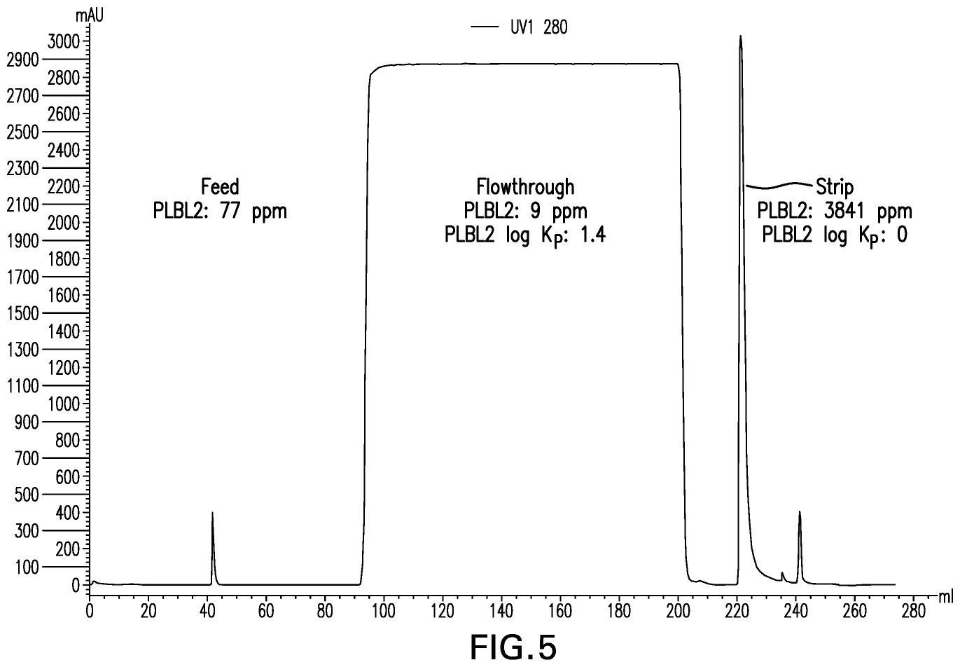

The column used was approximately 7 mL volume at a 20 cm bed height for POROS HQ resin. The mAb3 feed was at 13.5 mg/mL concentration in a Tris and acetate mixture at pH 7.5 with the acetate counterion at approximately 110 mM. The mAb3 log K_{P} at this condition is close to zero whereas the PLBL2 log K_{P} is around 1.4 (FIG. 1A), indicating that mAb3 will not but PLBL2 will bind to the resin to certain extent. The process was run in flowthrough mode, and the chromatogram indicates that very little mAb3 bound to the column (FIG. 5). PLBL2 in different fractions was quantified using a mass spectrometry method on a QE HF-X system with known PLBL2 peptides used to calibrate concentrations. The concentration of PLBL2 in the feed was 77 ppm. The concentration of PLBL2 in the flowthrough was 9 ppm. The concentration of PLBL2 in the strip was 3841 ppm. The detected amounts indicate that over 85% of PLBL2 was removed from mAb3 in the flowthrough pool. High amounts of PLBL2 in the strip pool indicate that the lipase bound to the resin under the flowthrough condition, as predicted by the log K_{P} value of 1.4, and was eluted under the 1 M NaCl high salt strip condition, also predicted by the log K_{P} value of 0.

Thus, protein A and AEX represent promising steps to remove PLBL2 at common operating conditions with log α values of −2 during protein A loading and of approximately 1.5 during AEX loading at flowthrough mode. Similar α values can be achieved with LPLA2 for ProA and even better separation up to approximately 1.7 for AEX loading in flowthrough mode.

For mAb3, additional clearance beyond the protein A and AEX processes was also desired, so log K_{P} maps were generated at similar conditions to those used for PLBL2 and LPLA2 in Example 3. These log K_{P} values were then used to calculate log α across the ranges screened to identify the conditions of greatest separation.

4.2 CEX Chromatography

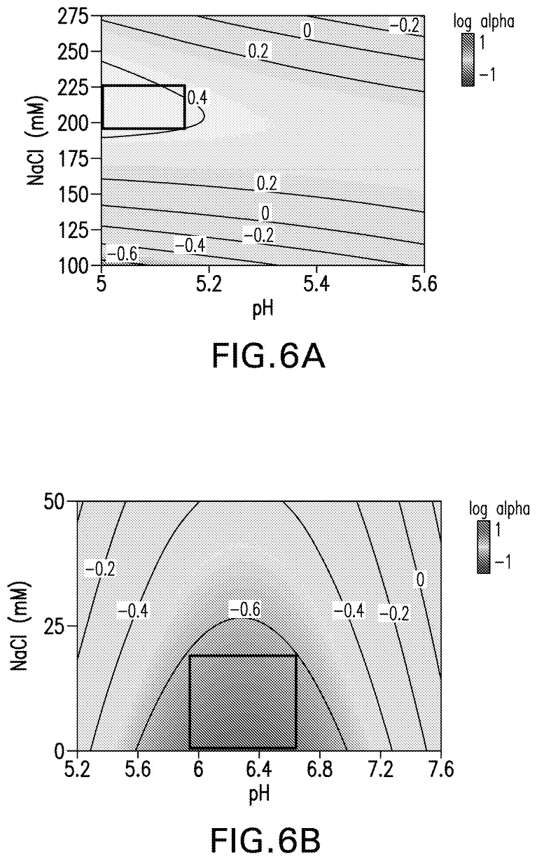

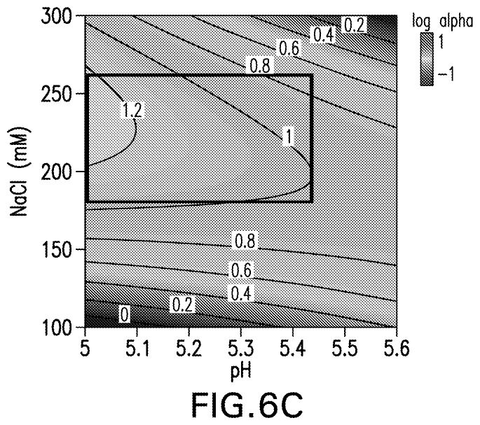

Conditions for separation using CEX chromatography with POROS 50 HS resin are depicted as log α values in FIGS. 6A and 6B for PLBL2 and in FIG. 6C for LPLA2 at the conditions listed in Table 3. For modulating binding with salt, the best separation conditions for PLBL2 were between pH 5-5.2 and 200-225 mM NaCl where log α values are approximately 0.4 (FIG. 6A, black box). Since the log α is positive, mAb3 is bound less strongly than PLBL2 indicating that, under these conditions, mAb3 would elute form the resin while PLBL2 might remain bound. The optimized area represents a somewhat narrow pH and salt range and does not have a particularly high log α. Confirmation using traditional column chromatography would still likely need to be performed for this process.

The separation for LPLA2 and mAb3 over these CEX conditions is much greater as shown in FIG. 6C. While the optimized range is similar to PLBL2, the a value for LPLA2 and mAb3 is greater than 1 at conditions from 200-250 mM NaCl and pH of 5.0-5.3.

In contrast to salt modulation, changing binding by pH produced negative log α values of approximately −0.6 (FIGS. 6B, black box). The region encompassing approximately pH 6.0-6.6 and less than 20 mM NaCl represents conditions where mAb3 bound more strongly to the resin than PLBL2. Thus, these conditions might be used as an intermediate wash where PLBL2 could be removed before eluting mAb3 at a higher pH (and/or higher salt).

To evaluate whether an additional CEX process can further separate PLBL2 and LPLA2 from mAb3 after an AEX process operated in flowthrough mode under the loading operating condition specified in Example 4.1, an AEX flowthrough pool was loaded on a CEX column containing POROS HS resin, operated in bind-and-elute mode with elution condition at pH 5.1 and 165 mM NaCl. The log α under this elution operating condition is 0.2 for PLBL2 (FIG. 6A) and 0.9 for LPLA2 (FIG. 6C). While these log α values are less than the value in the optimum range around 200 mM NaCl, the positive values still indicate that lipases will bind more strongly to the resin under this elution condition, particularly for LPLA2. Mass spectrometry analysis showed that the PLBL2 in the CEX feed was 5 ppm but the PLBL2 in the CEX elution pool dropped to 0.3 ppm. LPLA2 in the elution pool was below detection limits (data not shown). These results demonstrate that, under the elution operating condition specified, an additional CEX process can further separate PLBL2 from mAb3 after the AEX process run in flowthrough mode.

4.3 HIC

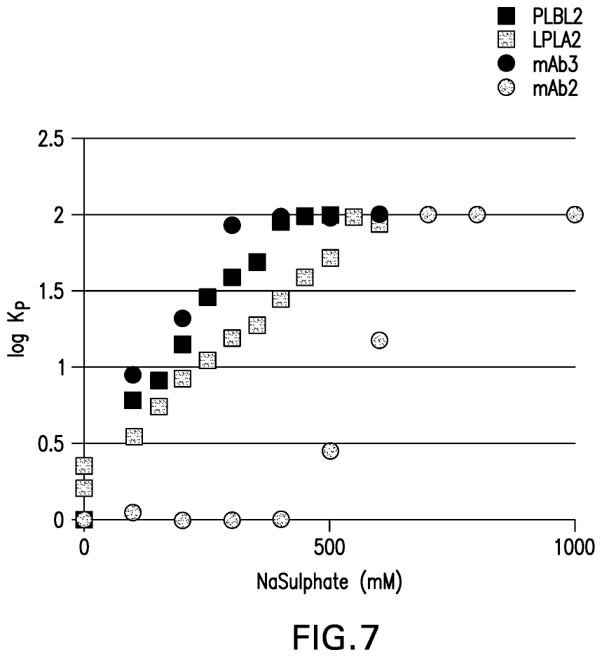

Partitioning of mAb3 and lipases was also compared on a HIC resin, Tosoh Butyl-650M, (FIG. 7) at the conditions listed in Table 3. Varying sodium sulfate concentration provides little separation between the mAb3 and PLBL2 with only 300 mM sodium sulfate providing any separation at all with a log α of approximately 0.3 at this condition. LPLA2 provides somewhat better separation with log α of about 0.5 between 300-400 mM sodium sulfate. In contrast, mAb2 is much less hydrophobic than mAb3, PLBL2, or LPLA2, and thus does not transition to strong binding to the HIC resin above log K_{P} of 1.5 until greater than 600 mM sodium sulfate. For mAb2 and PLBL2, log α values from 1.5-2.0 can be achieved between 300-500 mM sodium sulfate, a very wide salt range with promising separation capabilities for operating within. Similarly for LPLA2, log α values greater than 1 are seen in this same salt range.

4.4 Multimodal Chromatography

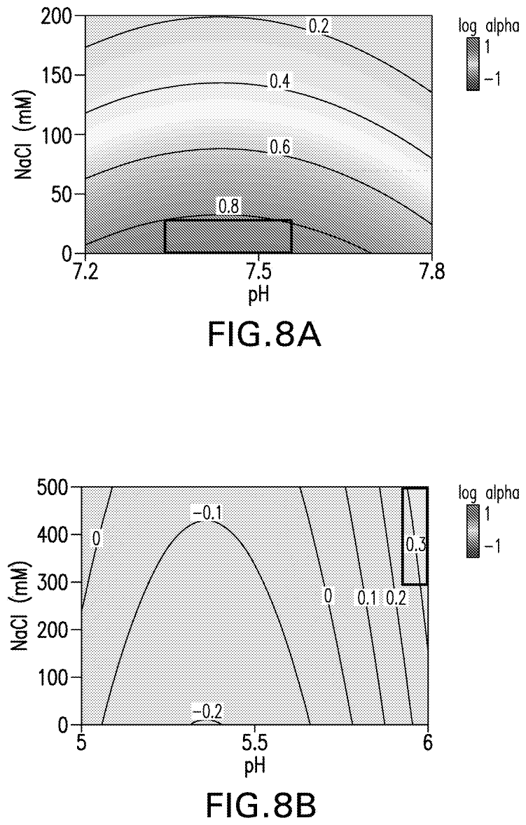

Partitioning of mAb3 and PLBL2 was finally compared for different multimodal resins, multimodal AEX resin, Capto adhere (FIG. 8A) and multimodal CEX resin, Capto MMC (FIG. 8B) at the conditions listed in Table 3.

For Capto adhere, PLBL2 bound strongly at all conditions (FIG. 4A), whereas mAb3 demonstrated hydrophobic interactions with stronger partitioning at higher salt. The resulting log α plot shows the highest separation factor (log α>0.8) under conditions of pH 7.3-7.6 at low salt where mAb3 binding was lowest (FIG. 8A). These conditions could be utilized for mAb3 elution or flowthrough while maintaining PLBL2 binding to the Capto adhere resin.

Capto MMC does not provide the same level of separation of mAb3 and PLBL2 as Capto adhere for the conditions screened. The best conditions were seen at pH 5.9-6.0 and above 300 mM NaCl, where log α values up to 0.3 were observed. Because the log α is greater than zero, these conditions might be used for mAb3 elution while maintaining PLBL2 binding (FIG. 8B). A wider or more favorable set of separation conditions might be possible at higher pH and/or higher salt because the optimal range was seen at the upper limits of both factors in this screen.

### Example 5: Optimization of CEX Operating Conditions for a Monoclonal Antibody mAb4 (MK-1308, Anti-CTLA4 Antibody)

Five factors were studied in the CEX Design of Experiment (DoE) as displayed in Table 4. Elution buffer pH and conductivity were adjusted using variable amounts of sodium acetate trihydrate, 4 M acetic acid, and sodium chloride. Load pH and conductivity were adjusted using 1 M Tris, 1 M acetic acid, and 1 M sodium chloride.

TABLE 4
CEX DoE factors to be studied

| | | Design Levels | | | | |
|---|---|---|---|---|---|---|
| | | Axial | | | | Axial |
| Factor | Units | Low | Low | Middle | High | High |
| Elution pH | — | 4.8 | 4.9 | 5.1 | 5.3 | 5.4 |
| Elution | mS/cm | 15 | 16 | 18 | 20 | 21 |
| Conductivity | | | | | | |
| Load pH | — | N/A | 4.9 | 5.1 | 5.3 | N/A |
| Load Conductivity | mS/cm | N/A | 4 | 6 | 8 | N/A |
| Resin Loading | g/L | 20 | 30 | 40 | 50 | 60 |

The DoE design came to a total of 40 runs as displayed in Table 5. Each elution buffer was prepared twice with the center-point elution buffer prepared three times.

TABLE 5
CEX DoE runs

| | | | Elution | | | | | |
|---|---|---|---|---|---|---|---|---|
| | | | Buffer | | Elution | | | |
| | | | Preparations | Elution | buffer | | Load | Resin |
| Run | | | (Whole | buffer | conductivity | Load | conductivity | load |
| (Randomized) | Standard | Type | Plots) | pH | (mS/cm) | pH | (mS/cm) | (g/L) |
| 1 | 32 | Overall Center | 1 | 5.1 | 18 | 5.1 | 6 | 40 |
| 2 | 31 | Overall Center | 1 | 5.1 | 18 | 5.1 | 6 | 40 |
| 3 | 10 | Factorial | 2 | 5.3 | 16 | 4.9 | 8 | 30 |
| 4 | 21 | Axial | 3 | 5.1 | 15 | 5.1 | 6 | 40 |
| 5 | 11 | Factorial | 4 | 5.3 | 16 | 5.3 | 4 | 30 |
| 6 | 16 | Factorial | 5 | 5.3 | 20 | 5.3 | 8 | 30 |
| 7 | 34 | Overall Center | 6 | 5.1 | 18 | 5.1 | 6 | 40 |
| 8 | 38 | Ext. Factorial | 7 | 4.8 | 21 | 5.1 | 6 | 40 |
| 9 | 7 | Factorial | 8 | 4.9 | 20 | 5.3 | 4 | 30 |
| 10 | 20 | Axial | 9 | 5.4 | 18 | 5.1 | 6 | 40 |
| 11 | 15 | Factorial | 10 | 5.3 | 20 | 5.3 | 4 | 50 |
| 12 | 9 | Factorial | 4 | 5.3 | 16 | 4.9 | 4 | 50 |
| 13 | 19 | Axial | 11 | 5.4 | 18 | 5.1 | 6 | 40 |
| 14 | 33 | Overall Center | 6 | 5.1 | 18 | 5.1 | 6 | 40 |
| 15 | 12 | Factorial | 2 | 5.3 | 16 | 5.3 | 8 | 50 |
| 16 | 29 | Axial | 12 | 5.1 | 18 | 5.1 | 6 | 20 |
| 17 | 18 | Axial | 13 | 4.8 | 18 | 5.1 | 6 | 40 |
| 18 | 5 | Factorial | 8 | 4.9 | 20 | 4.9 | 4 | 50 |
| 19 | 39 | Ext. Factorial | 14 | 5.4 | 15 | 5.1 | 6 | 40 |
| 20 | 40 | Ext. Factorial | 15 | 5.4 | 15 | 5.1 | 6 | 40 |
| 21 | 8 | Factorial | 16 | 4.9 | 20 | 5.3 | 8 | 50 |
| 22 | 4 | Factorial | 17 | 4.9 | 16 | 5.3 | 8 | 30 |
| 23 | 1 | Factorial | 18 | 4.9 | 16 | 4.9 | 4 | 30 |
| 24 | 13 | Factorial | 5 | 5.3 | 20 | 4.9 | 4 | 30 |
| 25 | 23 | Axial | 19 | 5.1 | 21 | 5.1 | 6 | 40 |
| 26 | 37 | Ext. Factorial | 20 | 4.8 | 21 | 5.1 | 6 | 40 |
| 27 | 3 | Factorial | 18 | 4.9 | 16 | 5.3 | 4 | 50 |
| 28 | 27 | Axial | 12 | 5.1 | 18 | 5.1 | 4 | 40 |
| 29 | 17 | Axial | 21 | 4.8 | 18 | 5.1 | 6 | 40 |
| 30 | 30 | Axial | 12 | 5.1 | 18 | 5.1 | 6 | 60 |
| 31 | 6 | Factorial | 16 | 4.9 | 20 | 4.9 | 8 | 30 |
| 32 | 26 | Axial | 12 | 5.1 | 18 | 5.3 | 6 | 40 |
| 33 | 22 | Axial | 22 | 5.1 | 15 | 5.1 | 6 | 40 |
| 34 | 25 | Axial | 12 | 5.1 | 18 | 4.9 | 6 | 40 |
| 35 | 14 | Factorial | 10 | 5.3 | 20 | 4.9 | 8 | 50 |
| 36 | 24 | Axial | 23 | 5.1 | 21 | 5.1 | 6 | 40 |
| 37 | 28 | Axial | 12 | 5.1 | 18 | 5.1 | 8 | 40 |
| 38 | 2 | Factorial | 17 | 4.9 | 16 | 4.9 | 8 | 50 |
| 39 | 35 | Overall Center | 24 | 5.1 | 18 | 5.1 | 6 | 40 |
| 40 | 36 | Overall Center | 24 | 5.1 | 18 | 5.1 | 6 | 40 |

Each run was operated according to the steps in Table 6 on a 7 mL chromatography column. The elution pools and strips of each run were collected for yield and quality analysis.

TABLE 6
CEX operating steps

| | | | Target | Flow | | |
|---|---|---|---|---|---|---|
| | | Target | Conductivity | Rate | Column | |
| Step | Buffer | pH | (mS/cm) | (cm/hr) | Volume | Flow |
| Sanitization | 0.5M NaOH | >12 | 83-105 | 300 | ≥3 | Down |
| Equilibration 1 | 1M NaCl | NA | 75-95 | 300 | ≥3 | Down |
| Equilibration 2 | 20 mM Sodium | 4.9-5.3 | ≤2.0 | 300 | ≥5 | Down |
| | Acetate | (DoE) | | | | |
| Load | CEX Column | 4.9-5.3 | 4-8 | 300 | ~5 | Down |
| | Load | (DoE) | (DoE) | | | |
| Wash | 20 mM Sodium | 4.9-5.3 | ≤2.0 | 300 | ≥3 | Down |
| | Acetate | (DoE) | | | | |
| Elution | 20 mM Sodium | 4.8-5.4 | 16-21 | 300 | ≥0.250 AU/cm | Down |
| | Acetate, 135- | (DoE) | (DoE) | | ≤1.5 AU/cm | |
| | 195 mM NaCl | | | | Total | |
| | (DoE) | | | | Approximate | |
| | | | | | CV of 2-6 | |
| Strip | 1M NaCl | NA | 75-95 | 300 | ≥3 | Down |
| Regeneration | 0.5M NaOH | >12 | 83-105 | 300 | ≥3 | Down |
| Storage | 0.1M NaOH | >12 | 18-24 | 300 | ≥3 | Down |

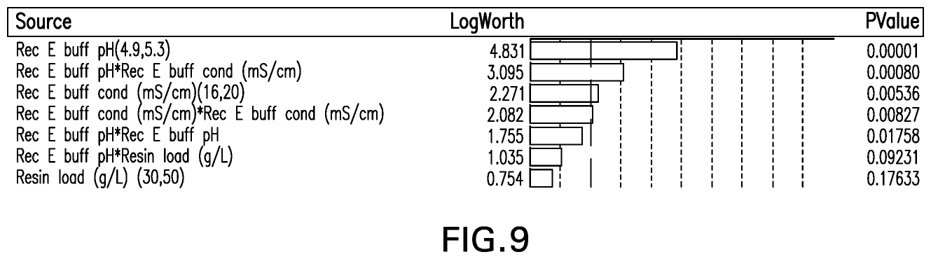

The elution pools and strips of each run were submitted for residual HCP ELISA analysis. Elution buffer pH and conductivity were found to have the greatest effect on residual HCP ELISA results (FIG. 9).

Hence, specific runs based on elution buffer conditions were analyzed further by liquid chromatography-mass spectrometry (LC-MS) (see Table 7). Due to material limitations, the pools and strips of some runs with the same elution buffer conditions were combined (e.g., 10&13 below).

TABLE 7
CEX DoE pools and strips submitted for LCMS analysis

| | | Elution | | | |
|---|---|---|---|---|---|
| | Elution | Buffer | | Load | Resin |
| | Buffer | Conductivity | Load | Conductivity | Loading |
| Run # | pH | (mS/cm) | pH | (mS/cm) | (g/L) |
| 10&13 | 5.4 | 18 | 5.1 | 6 | 40 |
| 19&20 | 5.4 | 15 | 5.1 | 6 | 40 |
| 11&24 | 5.3 | 20 | 5.1* | 4* | 40* |
| 12 | 5.3 | 16 | 4.9 | 4 | 50 |
| 25 | 5.1 | 21 | 5.1 | 6 | 40 |
| 1&2 | 5.1 | 18 | 5.1 | 6 | 40 |
| 34 | 5.1 | 18 | 4.9 | 6 | 40 |
| 37 | 5.1 | 18 | 5.1 | 8 | 40 |
| 40 | 5.1 | 18 | 5.1 | 6 | 40 |
| 18 | 4.9 | 20 | 4.9 | 4 | 50 |
| 27 | 4.9 | 16 | 5.3 | 4 | 50 |

*Average value: Run 11 had load condition: pH 5.3, cond 4 mS/cm @ 50 g/L loading
Run 24 had load condition: pH 4.9, cond 4 mS/cm @ 30 g/L loading

LC-MS results indicated no lipases present in the CEX pool of all the samples tested based on a database search of known peptide sequences. The detection limit of LC-MS in 1 mg DS was assessed by spiking in 48 different human proteins (6 to 83 kDa) ranging from 500 amoles to 50 pmoles. At least two unique peptides were identified for as low as 0.6 ppm spiked-in protein (data not shown).

Five lipases including phospholipase B-like 2 (PLBL2), lipoprotein lipase (LPL), phospholipase A2 XV (LPLA2), phospholipase A2 VII (LP-PLA2), and lysosomal acid lipase A (LAL/LIPA) were identified in each CEX strip sample and relatively quantified (see Table 8). These results suggest strong binding of lipases to the CEX resin in the elution buffer range tested (pH 4.9-5.4, conductivity 15-21 mS/cm, which corresponds to sodium chloride concentration of 135-195 mM).

TABLE 8
Relative quantification of endogenous lipases in mAb4 CEX Strips

| | PLBL2 | LPL | LPLA2 | LP-PLA2 | LAL |
|---|---|---|---|---|---|
| Run # | (ppm) | (ppm) | (ppm) | (ppm) | (ppm) |
| 10&13 | 41.39 | 55.22 | 1.39 | 1.82 | 82.86 |
| 19&20 | 19.37 | 27.44 | 0.68 | 0.88 | 41.66 |
| 11&24 | 113.20 | 171.50 | 4.20 | 6.99 | 260.38 |
| 12 | 60.74 | 89.43 | 2.09 | 2.45 | 107.26 |
| 25 | 80.92 | 114.81 | 2.87 | 4.10 | 192.71 |
| 1&2 | 17.69 | 24.00 | 0.52 | 0.74 | 33.04 |
| 34 | 29.76 | 39.83 | 1.09 | 1.65 | 65.75 |
| 37 | 37.18 | 47.43 | 1.19 | 1.69 | 81.12 |
| 40 | 31.52 | 41.89 | 1.04 | 1.47 | 64.53 |
| 18 | 18.99 | 22.13 | 0.68 | 1.04 | 39.29 |
| 27 | 2.00 | 5.85 | 0.12 | 0.07 | 9.25 |

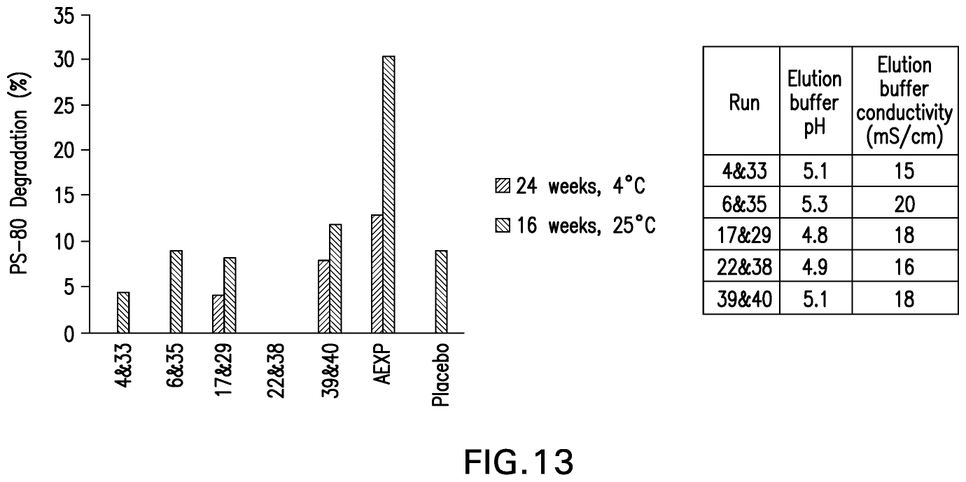

To confirm improvement of PS-80 stability in the elution buffer design space, a PS-80 stability study was conducted. To have sufficient mAb mass for the stability study, CEXPs from different runs (but same elution buffer condition) of Table 5 were combined by equal mAb mass ratios (1:1) and formulated. A PS-80 stability study was conducted on the combined CEXP runs at 5±3° C. and 25±3° C. for 24 weeks and 16 weeks, respectively (FIG. 13). This study demonstrated stable PS-80 for the duration of the study for all CEXP samples tested (<20% PS-80 degradation), while the positive control, AEXP, degraded ˜30% after 16 weeks at 25±3° C. The operating space for CEX elution buffer pH 5.1±0.2 and conductivity 18±2 mS/cm is demonstrably robust for improving PS-80 stability in the CEXP, and hence, DS.

### Example 6: PS-80 Stability Increased as Host Cell Lipases were Removed

PS-80 stability was assessed by measuring the PS-80 concentration of a solution containing PS80 over a specified time at specified temperatures. A significant change in PS-80 concentration is defined as two consecutive results outside of a ±0.02 mg/mL PS-80 concentration range (i.e., assay variability) compared to the time zero result.

The mAb4 drug substance (DS) is a PS-80-containing solution made via a formulation step, which entails the separate additions of the 49% (w/w) sucrose and 85 mM methionine stock solution, and 10% (w/w) PS-80 stock solution to achieve a final DS concentration of 50 mg/mL mAb4 in 10 mM Histidine buffer (pH 5.5), 10 mM Methionine, 7% (w/v) sucrose, and 0.02% (w/v) PS-80.

The PS-80 stability was compared between two mAb4 DS samples that were generated from a two-column and a three-column purification scheme. The two-column purification scheme included Protein A and AEX. The resulting AEX pool (AEXP) was formulated into DS and is referred to as “AEXP DS.” The three-column purification scheme included Protein A, AEX, and CEX. The resulting CEX pool (CEXP) was formulated into DS and is referred to as “CEXP DS.” Furthermore, a placebo containing the same DS formulation without protein was used as a negative control throughout the study.

Placebo, AEXP DS, and CEXP DS were filled into separate glass vials at a 2.2 ml fill volume and capped with a rubber stopper to simulate the storage of the drug product. Vials were placed in the following stability chambers:
5° C.±3° C.
25° C.±3° C., 60%±5% relative humidity (RH)
40° C.±2° C., 75%±5% relative humidity (RH)
   Samples were pulled and tested for PS-80 concentration at 2-week intervals up to 12 weeks and 6 months (26 weeks).

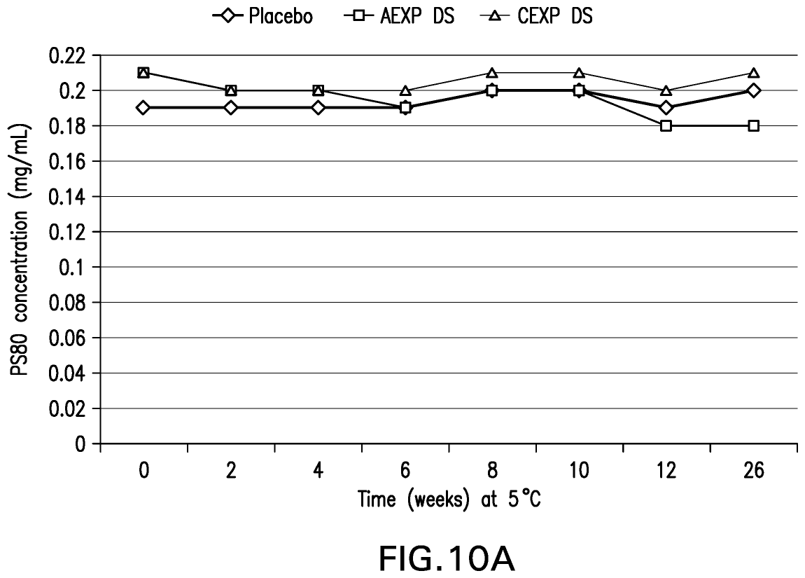

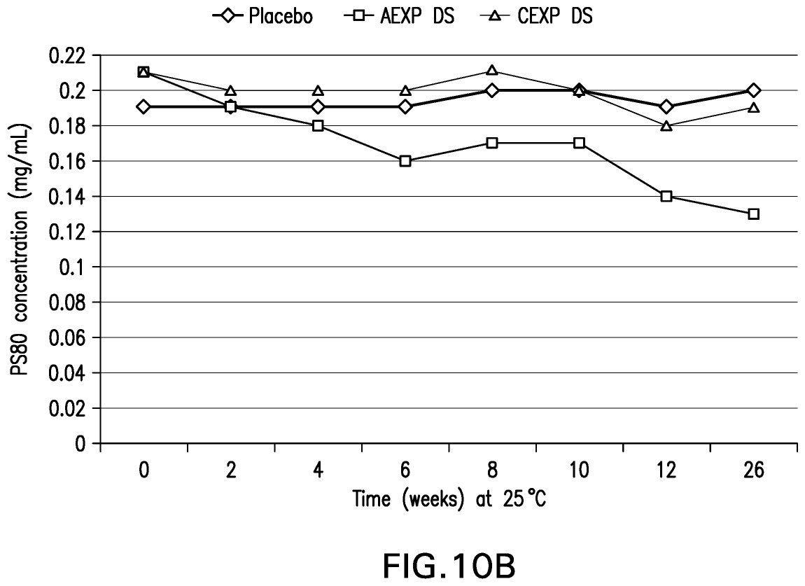

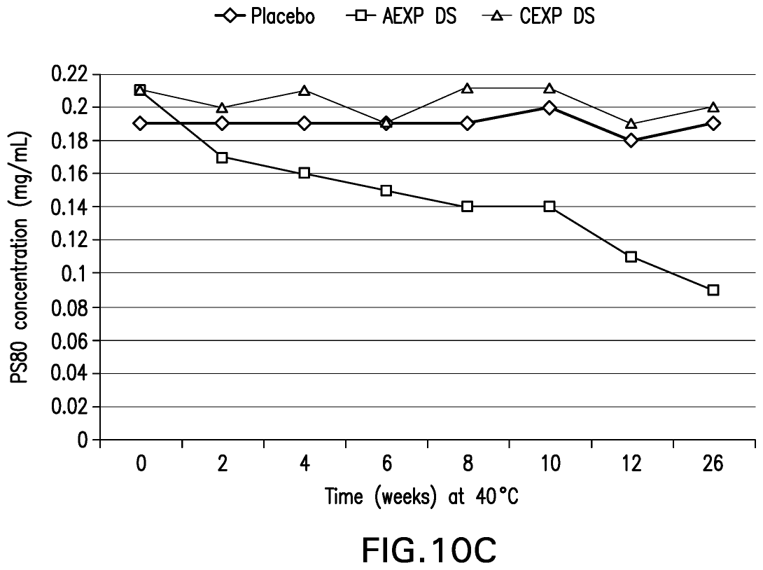

As shown in FIG. 10A, the PS-80 concentration in AEXP DS decreased from 0.21 (0 week) to 0.18 mg/mL (12 weeks) at 5° C. The degradation of PS-80 increased as the storage temperature increased. For example, at 25° C., the PS-80 concentration in AEXP DS decreased from 0.21 (0 week) to 0.18 mg/mL (4 weeks) and 0.15 mg/mL (26 weeks) (FIG. 10B); at 40° C., the PS-80 concentration in AEXP DS decreased from 0.21 (0 week) to 0.17 mg/mL (2 weeks) and 0.09 mg/mL (26 weeks) (FIG. 10C). On the other hand, the PS-80 concentration in CEXP DS did not change significantly over time under all three different temperatures, which is comparable to Placebo.

In parallel with this PS-80 stability study, the in-process intermediates of the same batch along with the corresponding chromatography strip samples were tested for lipase identification by liquid chromatography-mass spectrometry (LC-MS) (Table 9). PLBL2 and LPL were found in AEXP but absent from CEXP. Both PLBL2 and LPL were present in the AEX strip and CEX strip, suggesting strong binding of the lipases to the resin. Therefore, it is hypothesized that the presence of PLBL2 and LPL in the AEXP could be one potential cause for the PS-80 concentration decline at 5-40° C. in the AEXP DS. Adding a third CEX column can effectively remove these lipases and improve the PS-80 stability in the CEXP DS.

TABLE 9
Relative quantification of endogenous PLBL2 and LPL
in mAb4 process intermediates

| | PLBL2 (ppm) | LPL (ppm) |
|---|---|---|
| | Peptide: LTFPTGR | Peptide: LVAALYK |
| Sample | (SEQ ID NO: 34) | (SEQ ID NO: 35) |
| HCCF | 734 | 377.4 |
| PAP | 10.7 | 81.9 |
| PA FT 1 | 190.3 | 65 |
| FNVIP | 10.8 | 39.6 |
| AEXP | 1.2 | 4.7 |
| AEX Strip | 2408.3 | 3398.6 |
| CEXP | Not detectable | Not detectable |
| CEX Strip | 135.2 | 538.9 |
| DS | Not detectable | Not detectable |

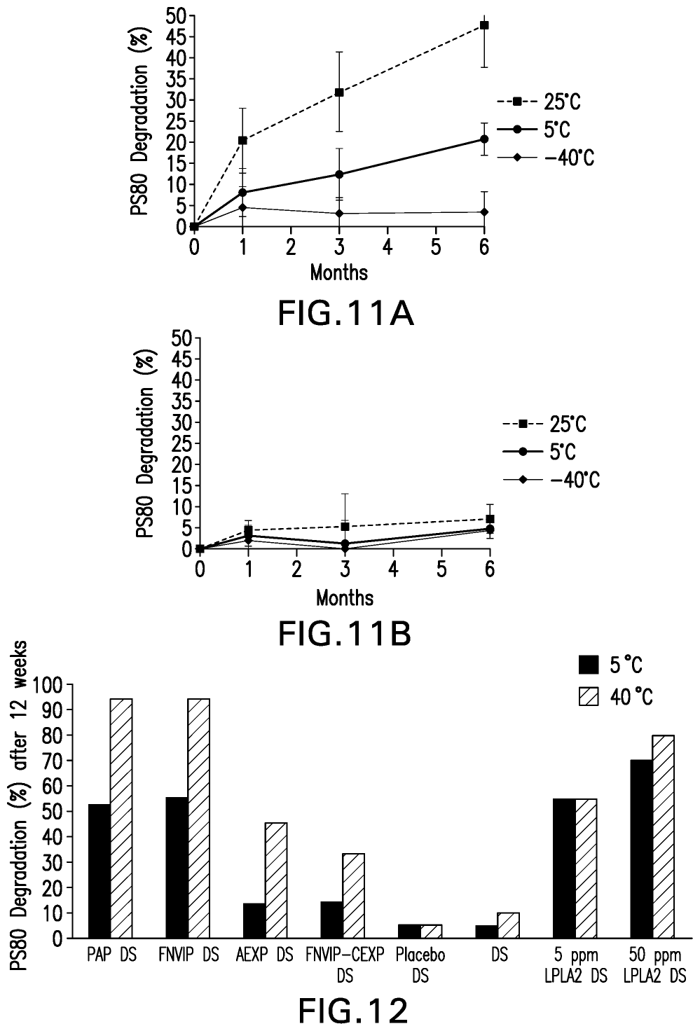

DS from four mAb4 purification batches utilizing only two chromatography steps (Protein A and AEX) were placed on a stability study at −40° C., 5° C., and 25° C. for up to six months (FIG. 11A). The average PS80 degradation at 25° C. was approximately 20% after just 1 month and approximately 45% after 6 months. DS from three mAb4 purification batches utilizing three chromatography steps (Protein A, AEX, and CEX) were placed on a stability study at −40° C., 5° C., and 25° C. for up to six months (FIG. 11B). The average PS80 degradation at 25° C. did not go above 10% after six months. These results further support the claim that CEX removes an impurity (such as lipases) and improves PS-80 stability.

To determine whether the AEX step may be skipped in the whole process, material following the Protein A step, specifically, filtered neutralized viral inactivated pool (FNVIP) was loaded on the CEX column. The resulting CEX pool was formulated into DS and named “FNVIP-CEXP DS.” Furthermore, one of the lipases identified in the CEX strip, LPLA2, was spiked into DS at two concentrations, 5 ppm and 50 ppm, as positive lipase controls. These DS samples, along with DS from protein A pool (PAP), FNVIP, AEXP, placebo (the same formulation without DS), and the three-column purification process (Protein A, AEX, and CEX), were placed on a stability study at 5° C. and 40° C. (FIG. 12). After 12 weeks at 40° C., PAP DS, FNVIP DS, and both LPLA2-spiked DS samples experienced over 50% PS80 degradation, while the placebo DS and standard three-column process DS remained at or below 10% PS80 degradation. These results indicate that lipases, such as LPLA2, cause PS80 degradation and are more concentrated in the PAP and FNVIP of the purification process. The AEXP DS and FNVIP-CEXP DS PS80 degraded approximately 30-40% after 12 weeks at 40° C., suggesting a three-column purification process (Protein A, AEX, and CEX) is necessary to fully remove the residual lipase from mAb4 DS.

For further verification that lipases are removed by CEX, process intermediate samples from a 500 L HCCF batch (19K-1308-19002) and 2000 L HCCF batch (W18-MK1308-010) were submitted for liquid chromatography—multiple reaction monitoring (LC-MRM). LC-MRM was conducted to quantify three lipases: phospholipase B-like 2 (PLBL2), phospholipase A2 XV (LPLA2), and lipoprotein lipase (LPL). See results from analysis in Table 7 below.

TABLE 10
Relative quantification of endogenous PLBL2,
LPLA2, and LPL in mAb4 process intermediates

| | PLBL2 | LPLA2 | LPL |
|---|---|---|---|
| Sample ID | (ng/mg) | (ng/mg) | (ng/mg) |
| 19K-1308-19002 PAP-2 | 30.2 | 1.8 | 42.9 |
| 19K-1308-19002 AEXL-1 | 26.4 | 1.8 | 39.7 |
| 19K-1308-19002 AEXP-1 | 3.1 | BLQ | 5.2 |
| 19K-1308-19002 CEXP-2 | BLQ | BLQ | BLQ |
| W18-MK1308-010 PAP-1 | 27 | 1.7 | 38.1 |
| W18-MK1308-010 AEXL-1 | 23.9 | 1.5 | 27.7 |
| W18-MK1308-010 CEXL-2 | 3.2 | BLQ | 5.9 |
| W18-MK1308-010 CEXP-2 | BLQ | BLQ | BLQ |

BLQ = below limit of quantification. BLQ ≤ 1 ng/mg

### Example 7: Lipase Spiking Study—Clearance of PLBL2, LPL, and Basic Variants Using MK-1308, an Anti-CTLA4 Antibody

This example objective was to understand the capability of an individual unit operation to remove process and product related impurities. Clearance of PLBL2 and LPL were assessed by spiking feeds with AEX concentrated strip and CEX concentrated strip for the respective steps. Lipase levels were determined using Multiple Reaction Monitoring-Mass Spectrometry (MRM-MS) lipase assay.

Anion exchange chromatography (AEX) utilizes POROS HQ50 resin. An anti-CTLA4 antibody is loaded at a target of 200 g product/L resin. The antibody flows through the column whereas impurities, such as host cell protein, HMW, and DNA, bind to the column. A portion of the AEX strip was diluted using a 1:1 volume ratio of 20 mM sodium acetate pH 5.1, concentrated using a 30 kDa regenerated cellulose ultrafiltration membrane, diafiltered, and pH adjusted to a target pH of 7.5 using 1M tris base. The concentrated and dialyzed AEX strip was spiked into AEXL for evaluating rHCP, lipase, and HMW clearance across the AEX step. CHO DNA concentrate and spiked into AEXL for evaluating rDNA clearance across the AEX step.

Cation exchange chromatography (CEX) utilizes POROS HS50 resin. An anti-CTLA4 antibody is loaded at a target of 40 g product/L resin. Impurities, such as HCP, HMW, and rProA ligand, are separated by the sodium chloride concentration in the elution buffer. A portion of the CEX strip was diluted using a 1:1 volume ratio of 20 mM sodium acetate pH 5.1, concentrated using a 30 kDa regenerated cellulose ultrafiltration membrane and diafiltered. The concentrated and dialyzed CEX strip was spiked into CEXL for evaluating rHCP, lipase, basic variants, and HMW clearance across the CEX step.

Mabselect Sure Protein A ligand was purchased from GE Healthcare, diluted to 0.2 g/L in DI water, and spiked into CEXL for evaluating rProA ligand clearance across the CEX step. All rProA ligand levels tested (up to 79 ppm) resulted in below LOQ (0.3 mg/ml) in CEXP.

AEX was run at lower feed pH and higher feed conductivity, while CEX was run at centerpoint conditions. The centerpoint conditions for AEX are: Load: pH 7.5, ≤5 mS/cm, 200 g/L, Wash: 25 mM Tris-HCl, pH 7.5. The centerpoint conditions for CEX are: Load: pH 5.1, ≤6 mS/cm, 40 g/L, Wash: 20 mM Sodium Acetate, pH 5.1, and Elute: 20 mM Sodium Acetate, pH 5.1, 18 mS/cm achieved by adding 165 mM NaCl.

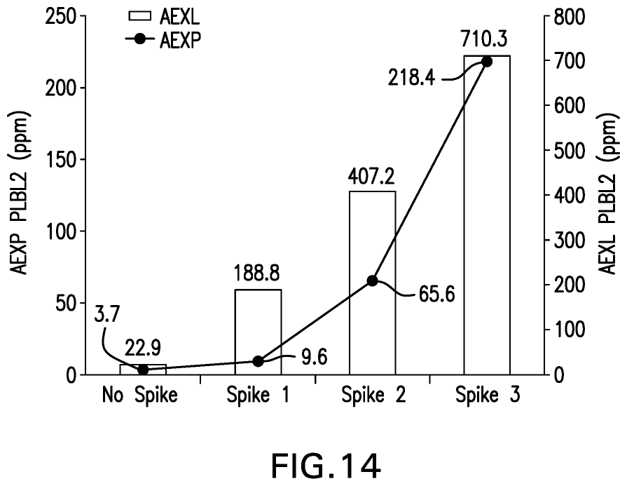

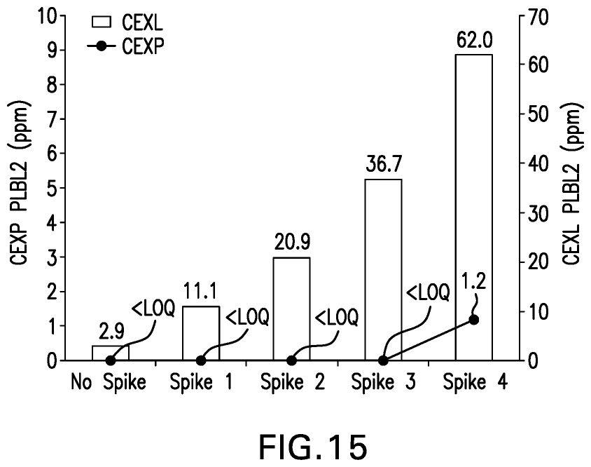

Feed and product PLBL2 results for each spiking level are shown in FIG. 14-FIG. 15. For almost every spiking level across CEX, PLBL2 was cleared below the Limit of Quantification (LOQ) of 1.0 ppm (FIG. 15). The maximum PLBL2 capacity for CEX was about 62 ppm (FIG. 15). For AEX, PLBL2 levels in AEXP increased exponentially as the PLBL2 spike levels in the AEXL increased (FIG. 14). The maximum PLBL2 capacity for AEX was about 350-400 ppm (FIG. 14).

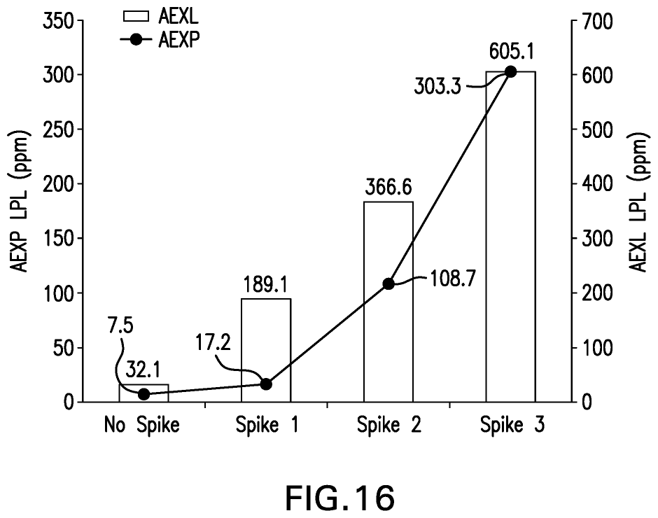

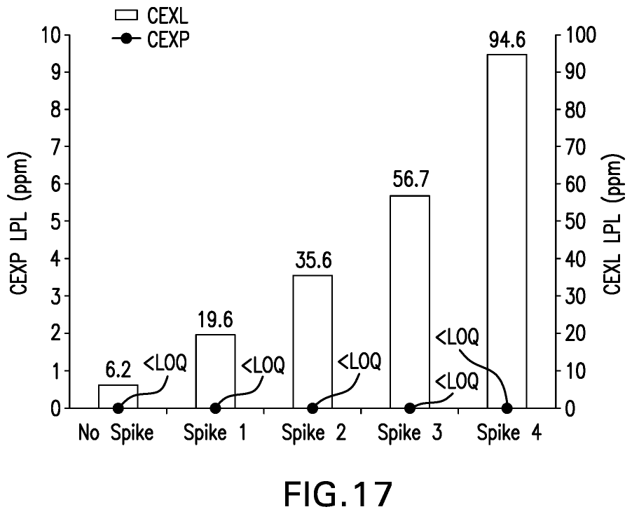

Feed and product LPL results for each spiking level are shown in FIG. 16-FIG. 17. For every spiking level across CEX, both LPL was cleared below the LOQ of 1.0 ppm (FIG. 17). The maximum LPL capacity for CEX was about 94.6 ppm (FIG. 17). For AEX, LPL levels in AEXP increased exponentially as the LPL spike levels in the AEXL increased (FIG. 16). The maximum LPL capacity for AEX was about 300-350 ppm (FIG. 16).

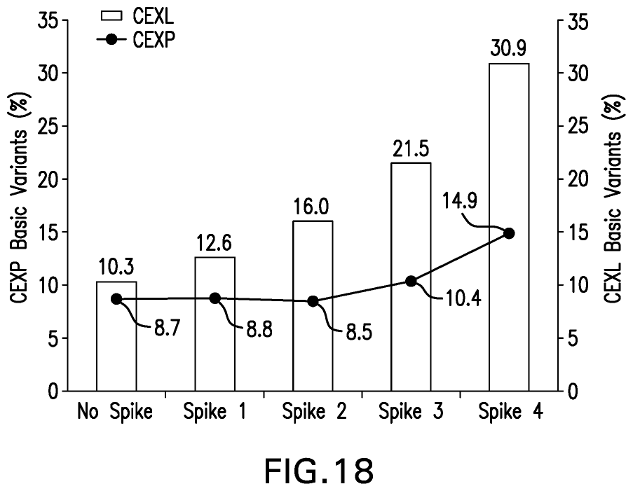

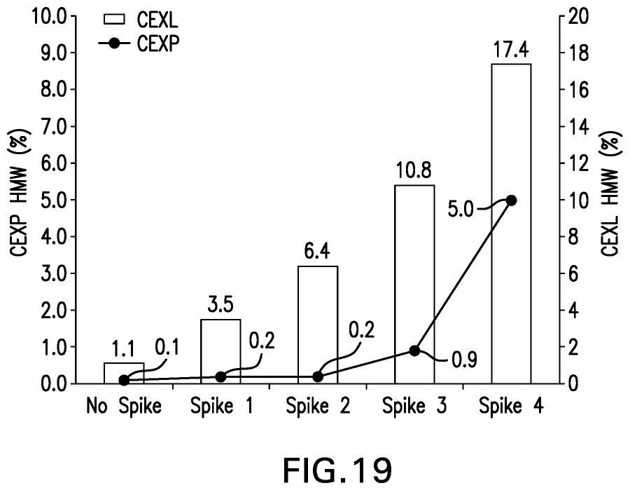

Clearance of basic variants was studied across CEX at center point conditions by spiking feeds with CEX concentrated strip at various levels and measuring resulting basic variant levels using HP-IEX. Feed and product basic variants results for each spiking level are shown in FIG. 18. The basic variant level remained constant in the CEXP for the first two impurity spike levels (FIG. 18), which corresponded to a maximum HMW level of about 6.4% (FIG. 19). The basic variants increased linearly in the CEXP in the third and fourth spike levels (FIG. 18). The maximum capacity for basic variants for CEX would be 30.9% (FIG. 18) but the HMW level in the CEXP was 5.0% (FIG. 13), which would not meet projected commercial DS specifications of about 2%. Therefore, the maximum capacity for basic variants for CEX was 21.5% (FIG. 18).

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

TABLE 11
a brief description of the sequences in the sequence listing.

| SEQ ID | |
|---|---|
| NO: | Description |
| 1 | Ipilimumab CDRL1 |
| 2 | Ipilimumab CDRL2 |
| 3 | Ipilimumab CDRL3 |
| 4 | Ipilimumab CDRH1 |
| 5 | Ipilimumab CDRH2 |
| 6 | Ipilimumab CDRH3 |
| 7 | Ipilimumab Heavy Chain VR |
| 8 | Ipilimumab Light Chain VR |
| 9 | Ipilimumab Mature Heavy Chain |
| 10 | Ipilimumab Mature Light Chain |
| 11 | CTLA4 Ab heavy chain CDR1 |
| 12 | CTLA4 Ab heavy chain CDR2 |
| 13 | CTLA4 Ab heavy chain CDR3 |
| 14 | CTLA4 Ab light chain CDR1 |
| 15 | CTLA4 Ab light chain CDR2 |
| 16 | CTLA4 Ab light chain CDR3 |
| 17 | CTLA4 Ab light chain CDR3 |
| 18 | CTLA4 Ab light chain CDR3 |
| 19 | 8D2/8D2 (RE) heavy chain variable |
| 20 | 8D2/8D2 (RE) light chain variable |
| 21 | 8D2HIL1 heavy chain variable |
| 22 | 8D2H1L1 light chain variable |
| 23 | 8D2H2L2 heavy chain variable |
| 24 | 8D2H2L2 light chain variable |
| 25 | 8D2H2L2 variant 1 heavy chain variable |
| 26 | 8D3H3L3 heavy chain variable |
| 27 | 8D3H3L3 light chain variable |
| 28 | 8D2H2L15 heavy chain variable |
| 29 | 8D2H2L15 light chain variable |
| 30 | 8D2H2L17 heavy chain variable |
| 31 | 8D2H2L17 light chain variable |
| 32 | 8D2H2L2 VARIANT 1 Full Heavy Chain |
| 33 | 8D2H2L2 VARIANT 1 Full Light chain |
| 34 | PLBL2 Peptide |
| 35 | LPL Peptide |

1. A composition comprising
(i) about 10 mg/ml to about 200 mg/ml of an anti-CTLA4 antibody, or antigen binding fragment thereof;
(ii) about 5 mM to about 20 mM buffer;
(iii) about 6% to 8% weight/volume (w/v) non-reducing sugar;
(iv) about 0.01% to about 0.10% non-ionic surfactant;
(v) about 1 mM to about 20 mM anti-oxidant, and
(vi) wherein the level of PLBL2, LPLA2, and LPL are ≤1 ng/ml of CTLA4 antibody.

2. A composition comprising
(i) about 10 mg/ml to about 200 mg/ml of an anti-CTLA4 antibody, or antigen binding fragment thereof;
(ii) about 5 mM to about 20 mM buffer;
(iii) about 6% to 8% weight/volume (w/v) non-reducing sugar;
(iv) about 0.01% to about 0.10% non-ionic surfactant;
(v) about 1 mM to about 20 mM anti-oxidant; and
(vi) a residual amount of host cell lipase, wherein the residual amount of host cell lipase is less than 2 ppm.

3. A composition comprising an anti-CTLA4 monoclonal antibody or antigen binding fragment thereof, a residual amount of host cell lipase, and polysorbate-80 wherein the composition comprises a stable polysorbate-80 (PS-80) concentration, wherein the stable PS-80 concentration remains at or below 10% PS-80 degradation.

4. The composition of claim 3, wherein the lipase is selected from the group consisting of phospholipase B-like 2 (PLBL2), lipoprotein lipase (LPL), lysosomal phospholipase A2 (LPLA2), phospholipase A2 VII (LP-PLA2), and lysosomal acid lipase A (LAL).

5. The composition of claim 4, wherein the level of PLBL2, LPLA2, and LPL are ≤1 ng/mg.

6. The composition of claim 3, wherein the composition comprises:
(i) less than 1 ng PLBL2/mg of CTLA4 antibody,
(ii) less than 1 ng of LPLA2/mg of CTLA4 antibody,
(iii) less than 1 ng of LPL/mg of CTLA4 antibody,
(iv) less than 1 ng of LP-PLA2/mg of CTLA4 antibody, or
(v) less than 1 ng of LAL/mg of CTLA4 antibody.

7. (canceled)

8. (canceled)

9. (canceled)

10. (canceled)

11. The composition of claim 3, wherein the PS-80 degradation is measured by the amount of intact PS-80 molecules using various methods comprising mass spectrometry (MS), liquid chromatography-mass spectrometry (LCMS), or solid phase extraction (SPE) on a HPLC system with a charged aerosol detector (CAD).

12. The composition of claim 3, wherein the PS-80 degradation is measured by the amount of degraded products using various methods comprising mass spectrometry (MS), liquid chromatography-mass spectrometry (LCMS), or solid phase extraction (SPE) on a HPLC system with a charged aerosol detector (CAD).

13. The composition of claim 3, wherein the composition comprises (i) about 10 mg/ml to about 200 mg/ml of an anti-CTLA4 antibody, or antigen binding fragment thereof.

14. The composition of claim 3, wherein the composition comprises 25 mg/ml of an anti-CTLA4 antibody, or antigen binding fragment thereof.

15. The composition of claim 3, wherein the composition comprises 50 mg/ml of an anti-CTLA4 antibody, or antigen binding fragment thereof.

16. The composition of claim 3, wherein the composition comprises (i) about 10 mg/ml to about 200 mg/ml of an anti-CTLA4 antibody, or antigen binding fragment thereof; (ii) about 5 mM to about 20 mM buffer (iii) about 6% to 8% weight/volume (w/v) non-reducing sugar; (iv) about 0.01% to about 0.10% PS-80; and (v) about 1 mM to about 20 mM anti-oxidant, wherein the anti-oxidant is methionine.

17. The composition of claim 3, wherein the PS-80 concentration remains±0.02 mg/mL as compared to the time zero result.

18. The composition of claim 3, wherein the PS-80 degradation remains at or below 10% for at least six months at 25° C.

19. The composition of claim 16, wherein the buffer is L-histidine buffer or sodium acetate buffer, and wherein the non-reducing sugar is sucrose.

20. (canceled)

21. (canceled)

22. The composition of claim 3, wherein the anti-CTLA4 antibody comprises:
i. light chain CDRs comprising a sequence of amino acids as set forth in SEQ ID Nos: 14, 15, and 16 and heavy chain CDRs comprising a sequence of amino acids as set forth in SEQ ID Nos: 11, 12, and 13;
ii. light chain CDRs comprising a sequence of amino acids as set forth in SEQ ID Nos: 14, 15, and 17 and heavy chain CDRs comprising a sequence of amino acids as set forth in SEQ ID Nos: 11, 12, and 13; or
iii. light chain CDRs comprising a sequence of amino acids as set forth in SEQ ID Nos: 14, 15, and 18 and heavy chain CDRs comprising a sequence of amino acids as set forth in SEQ ID Nos: 11, 12, and 13.

23. The composition of claim 3, wherein the anti-CTLA4 antibody comprises:
a. a heavy chain variable region comprising a sequence of amino acids as set forth in SEQ ID NO: 19 and a light chain variable region comprising a sequence of amino acids as set forth in SEQ ID NO:20;
b. a heavy chain variable region comprising a sequence of amino acids as set forth in SEQ ID NO: 21 and a light chain variable region comprising a sequence of amino acids as set forth in SEQ ID NO: 22;
c. a heavy chain variable region comprising a sequence of amino acids as set forth in SEQ ID NO: 23 and a light chain variable region comprising a sequence of amino acids as set forth in SEQ ID NO:24;
d. a heavy chain variable region comprising a sequence of amino acids as set forth in SEQ ID NO: 25 and a light chain variable region comprising a sequence of amino acids as set forth in SEQ ID NO:24;
e. a heavy chain variable region comprising a sequence of amino acids as set forth in SEQ ID NO: 26 and a light chain variable region comprising a sequence of amino acids as set forth in SEQ ID NO:27;
f. a heavy chain variable region comprising a sequence of amino acids as set forth in SEQ ID NO: 28 and a light chain variable region comprising a sequence of amino acids as set forth in SEQ ID NO:29; or
g. a heavy chain variable region comprising a sequence of amino acids as set forth in SEQ ID NO: 30 and a light chain variable region comprising a sequence of amino acids as set forth in SEQ ID NO:31.

24. The composition of claim 3, wherein the composition is purified by a process comprising a first protein A chromatography step, a second anion exchange chromatography step, and a third cation exchange step thereby producing a purified composition.

25. (canceled)

26. The composition of claim 4, wherein the composition comprises less than 1 ng/mg of PLBL2.

27. (canceled)

28. (canceled)

29. (canceled)

30. (canceled)

31. (canceled)

32. (canceled)

33. The composition of claim 3, wherein the polysorbate-80 concentration remains±0.02 mg/mL as compared to the time zero result.

34. The composition of claim 3, wherein PS-80 degradation remains at or below 10% for at least six months at 25° C.

35. (canceled)

36. (canceled)

37. (canceled)

38. (canceled)

39. (canceled)

40. (canceled)

41. A purified anti-CTLA4 monoclonal antibody composition, wherein the composition of claim 1 is purified by a process comprising a first protein A chromatography step, a second anion exchange chromatography step, and a third cation exchange step thereby producing a purified composition, wherein the amount of hamster PLBL2 is less than 1 ng/mg, and the composition comprises a stable polysorbate-80 concentration, wherein the stable PS80 concentration remains at or below 10% PS-80 degradation.

42. A method for improving PS-80 stability in a composition, wherein the composition of claim 1 is purified by a purification process comprising three chromatography steps consisting of: protein A, anion exchange, and cation exchange, and wherein the composition comprises (i) about 10 mg/ml to about 200 mg/ml of an anti-CTLA4 antibody, or antigen binding fragment thereof; (ii) about 5 mM to about 20 mM buffer (iii) about 6% to 8% weight/volume (w/v) non-reducing sugar; (iv) about 0.01% to about 0.10% non-ionic surfactant; and (v) about 1 mM to about 20 mM anti-oxidant, wherein the level of PLBL2 is ≤1 ng per mg of anti-CTLA4 antibody.

43. A purified anti-CTLA4 monoclonal antibody composition, wherein the composition of claim 2 is purified by a process comprising a first protein A chromatography step, a second anion exchange chromatography step, and a third cation exchange step thereby producing a purified composition, wherein the amount of hamster PLBL2 is less than 1 ng/mg, and the composition comprises a stable polysorbate-80 concentration, wherein the stable PS80 concentration remains at or below 10% PS-80 degradation.

44. A method for improving PS-80 stability in a composition, wherein the composition of claim 2 is purified by a purification process comprising three chromatography steps consisting of: protein A, anion exchange, and cation exchange, and wherein the composition comprises (i) about 10 mg/ml to about 200 mg/ml of an anti-CTLA4 antibody, or antigen binding fragment thereof; (ii) about 5 mM to about 20 mM buffer (iii) about 6% to 8% weight/volume (w/v) non-reducing sugar; (iv) about 0.01% to about 0.10% non-ionic surfactant; and (v) about 1 mM to about 20 mM anti-oxidant, wherein the level of PLBL2 is ≤1 ng per mg of anti-CTLA4 antibody.
